(19) United States
(12) Patent Application Publication
(10) Pub. No.: US 2011/0136137 A1
(43) Pub. Date: Jun. 9, 2011

(54) SERUM PROTEOMIC FOR FINDING DIAGNOSTIC MARKERS AND FOR MONITORING THERAPEUTICAL INTERVENTION IN TREATMENT OF HEPATOCELLULAR CARCINOMA

(76) Inventors: Jurgen Borlak, Lehrte OT Immensen (DE); Guiseppe Gazzana, Hannover (DE)

(21) Appl. No.: 12/744,977

(22) PCT Filed: Dec. 11, 2008
(86) PCT No.: PCT/EP08/10824

§ 371 Date: Dec. 20, 2010

(30) Foreign Application Priority Data
Dec. 13, 2007 (EP) 07076092.1

(51) Int. Cl. G01N 33/573; C12N 9/26; C07K 14/775; C12N 9/18; C07K 14/47; C07K 14/75; C12N 9/08; C12Q 1/40; C12Q 1/44; C12Q 1/28; G01N 33/68; G01N 33/92; C12Q 1/37

(52) U.S. Cl. 435/7.4; 435/201; 530/359; 435/197; 530/395; 530/382; 435/192; 530/386; 530/380; 530/350; 530/396; 435/22; 435/19; 435/28; 436/86; 436/87; 436/71; 436/501; 435/23

(57) ABSTRACT

The invention is directed to biomarkers for determining the EGFR kinase activity in a subject, and the use thereof for predicting and monitoring therapeutic intervention in cancer patients. Areas of application are the life sciences: biology, biochemistry, biotechnology, medicine and medical technology.

The biomarkers are selected from a first group consisting of Amy 1, Apo Al, Carbx, Casp, AFP, ApoM, SAP, Fib-a, Fib-b, Fib-g, ApoE, A2MG, A2MG isoform, Serpin, Clusterin, MHC-fB, SAP isoform, or from a second group consisting of Gpx3, properidin, MUP1, HMW-K, Lifr-p, Orm 1, MBL-A, MBP-C, wherein the biomarkers are regulated by EGF overexpression in a subject.

### CROSS-REFERENCE TO RELATED APPLICATIONS

This application is the United States National Phase under 35 U.S.C. §371 of PCT International Application No. PCT/EP2008/010824, filed on Dec. 11, 2008, and claiming priority to European Application No. 07076092.1, filed on Dec. 13, 2007. Those applications are incorporated by reference herein.

### INCORPORATION BY REFERENCE OF ELECTRONICALLY-SUBMITTED SEQUENCE LISTING

This application is accompanied by a sequence listing file submitted in electronic format and incorporated by reference herein. That sequence listing file was created on Dec. 19, 2010, is named 12744977sequence.txt, and has a file size of 770,748 bytes (753 KB).

### BACKGROUND OF THE INVENTION

1. Field of the Invention

The invention is directed to biomarkers for determining the EGFR kinase activity in a subject, and the use thereof for predicting and monitoring therapeutic intervention in cancer patients. Areas of application are the life sciences: biology, biochemistry, biotechnology, medicine and medical technology.

2. Background of the Related Art

The epidermal growth factor receptor (EGFR) plays an important role in various tumor diseases. Its hyperactivity can cause cancer of numerous organs, e.g. epithelial tumors of the lung, colon, breast, head and neck, ovarian, and liver (HCC). The hepatocellular carcinoma (HCC), for instance, ranks fifth among the most common malignant tumors worldwide and is the third leading cause of cancer-related deaths. Thereby, the number of HCC diseases in Europe and the United States steadily increases, as exemplarily demonstrated in the north east of Germany, where the number of HCC-incidences of males arose from 3.6% in 1976 to 5.7% in 2002. Chronic liver disease, which lead to cirrhosis and infection with hepatitis C are important risk factors for HCC. Overall, the formation of HCC is a multistage process, whereby the latest findings from cancer research evaluate the dysregulation of tumor progenitor genes as a predisposing or initial event leading to an epigenetic modification of progenitor or stem cells. Then, the risk of malignant transformation becomes dramatically increased by mutations in such tumor suppressor- and proto-oncogenes. Finally, the malignant transformation and the invasive tumor growth is massively promoted by epigenetic instability. Despite numerous findings the molecular basis of hepatocellular carcinoma remains insufficiently known.

Recently, the consequences of excessive EGF-signal transmission in liver cancer has been reported. This growth factor binds to and induces EGFR activity. Activated EGFR phosphorylates many proteins which are networked by signal transduction chains and therefore favour the emergence and further development of tumor malignancies. In particular, various tyrosine residues are phosphorylated, which subsequently act as docking sites for a number of proteins. In this way, EGFR is involved in diverse signaling pathways, including the mitogen-activated protein kinases (MAPK). This in turn has implications for the fate of the cell, leading to exaggerated proliferation, the lack of differentiation and migration of transformed tumor cells.

Since the EGF-receptor tyrosine kinase (RTK) plays a key role in malignant tumor diseases, therapeutic antibodies and small molecules, also termed as “EGFR kinase modulators”, that counter an increased activity of EGFR are frequently used for the treatment of cancer. However, besides to the EGF-signalling cascade further signalling pathways are involved in the formation and growth of cancer such as, e.g., Wnt-β-Catenin, Hedgehog and other receptor tyrosine kinases.

Thus, there is a need for new prognostic and predictive biomarkers and methods for easily identifying the indication “EGFR hyperactivity” or “EGFR overexpression”, respectively, in a subject suffering from or being susceptible to cancer, for accurately predicting and monitoring the response of the subject to a treatment with EGFR modulators, thereby enabling an individualized cancer treatment of the subject for enhancing its chances of survival.

### BRIEF SUMMARY OF THE INVENTION

The aim of the present invention is therefore to provide biomarkers, compositions and a kit, as well as a method for a fast, easy and efficient qualification or quantification of the EGFR kinase activity status of a subject suffering from or being susceptible to cancer, in particular for predicting and monitoring the response of a cancer patient to the treatment with an EGFR activity modulator.

The invention is based on the surprising finding that biomarkers selected from a first group consisting of

Amy 1, Apo Al, Carbx, Casp, AFP, ApoM, SAP, Fib-a, Fib-b, Fib-g, ApoE, A2MG, A2MG isoform, Serpin, Clusterin, MHC-fB, SAP isoform,
or from a second group consisting of
Gpx3, properidin, MUP1, HMW-K, Lifr-p, Orm 1, MBL-A, MBP-C,
are regulated by EGF overexpression in subjects suffering from or being susceptible to cancer.

The biomarkers according to the invention concern gene products of mammalia, preferably gene products of the genome of mus musculus or homo sapiens, in particular the respective gene products of homo sapiens are preferred.

Within the context of the invention, the term “subject” is directed to a mammal, in particular to a mouse or a human being suffering from or being susceptible to cancer, more particular to a human cancer patient or a transgenic cancer mouse, such as a HCC patient or a EGF-transgenic mouse may be.

The invention further concerns a composition for qualifying the EGFR kinase activity in a subject suffering from or being susceptible to cancer, in particular by an in vitro body fluid analysis, wherein the composition comprises an effective amount of at least one biomarker selected from the first group of said biomarkers or an effective amount of at least one biomarker selected from the second group of said biomarkers.

In one embodiment of the invention, the biomarker is preferably selected from a first group consisting of

Amy 1, Apo Al, Carbx, Casp, Fib-a, Fib-b, Fib-g, Clusterin, MHC-fB, SAP isoform

or from a second group consisting of

### HMW-K, Lifr-p, Orm 1, MBL-A, MBP-C.

In another preferred embodiment, the biomarker is selected from a first group consisting of

### AFP, ApoE, ApoM,

or from a second group consisting of
Gpx3, A2MG, A2MG isoform, SAP.

In particular, it is preferred, if the composition according to the invention comprises an effective amount of at least one biomarker selected from the first group of said biomarkers and an effective amount of at least one biomarker selected from the second group of said biomarkers, wherein the combination
(a) biomarker selected from the group consisting of Amy 1, Apo Al, Carbx, Casp, Fib-a, Fib-b, Fib-g, Clusterin, MHC-fB, SAP isoform and biomarker selected from the group consisting of HMW-K, Lifr-p, Orm 1, MBL-A, MBP-C or the combination
(b) biomarker selected from the group consisting of AFP, ApoE, ApoM and biomarker selected from the group consisting of Gpx3, A2MG, A2MG isoform, SAP
   is particularly preferred.

In another preferred embodiment the composition further comprises an effective amount of a biomarker selected from the group of EGF, thus allowing an easy calibration of the system.

In yet another preferred embodiment, the composition according to the invention further comprises an effective amount of a protease, in particular of trypsin, thus enabling a further enhancement of the system sensitivity.

The composition according to the invention, in particular the protease digest thereof, may be preferably used for producing a vaccine for the immunization of an animal in order to produce polyclonal antibodies specific for the at least one biomarker.

Another aspect of the invention concerns the use of the composition according to the invention for the production of a diagnostic agent, in particular of a diagnostic standard for in vitro body fluid analyses.

### BRIEF DESCRIPTION OF THE FIGURES

FIG. 1. A: Western blot representing the overexpression of EGF in livers of transgenic mouse model of HCC. (T=tumor samples; C=control samples). B: Histopathology of advanced liver cancer. C: Histopathology of control liver.

FIG. 2. A: Example for a map of the mouse serum proteome. (pH 3-10 NL; stain: coomassie blue; loaded sample: 500 μg); B, C: Zoom in for control and tumour serum samples, respectively. Immunoglobulins (circle) and glutathione peroxidase 3 (small rectangle) are down regulated in HCC-mice sera (right side of the panel). Two spots of serum amyloid component P (big rectangle) are up regulated (right side of the panel).

FIG. 3. ppm values of the 25 regulated proteins. A: down regulated proteins; C, E: up regulated proteins; B, D, F: proteins virtually present only in tumor samples; G: proteins virtually present only in control samples.

FIG. 4. Western blots of fibrinogen gamma (A), apoM (B), alfafetoprotein (C) and serum amyloid component P (D). All these proteins were up-regulated in serum samples of tumor bearing mice. (C=control, T=tumor).

### DETAILED DESCRIPTION OF THE INVENTION

The term “body fluid” according to the invention is directed to any body fluid of a subject, in particular to blood, plasma, serum or urine, whereas serum is the preferred body fluid within the context of the invention.

The term “diagnostic agent” as used herein relates to any solution, suspension or solid formulation, containing said composition in an acceptable amount for diagnostic purposes.

In particular, the composition is used for the production of a diagnostic agent for qualifying the EGFR kinase activity in a subject suffering from or being susceptible to cancer, preferably cancer of the liver, lung, breast, colon, prostate, bladder, head and neck, ovary or brain, in particular in a subject suffering from or being susceptible to HCC.

In a further preferred embodiment, the composition according to the invention is used for the production of a diagnostic agent for predicting or monitoring the response of a cancer patient to a method of treating cancer comprising administering an EGFR kinase modulator to the patient.

In yet another aspect, the invention provides a kit for qualifying the EGFR kinase activity in a subject suffering from or being susceptible to cancer, in particular for predicting or monitoring the response of a cancer patient to a method of treating cancer comprising administering an EGFR kinase modulator, wherein the kit comprises at least one standard (1) indicative of the body fluid level of a biomarker selected from the first group of said biomarkers in normal individuals or individuals having cancer associated with increased EGFR kinase activity and/or at least one standard (2) indicative of the body fluid level of a biomarker selected from the second group of said biomarkers in normal individuals or individuals having cancer associated with increased EGFR kinase activity, and instructions for the use of the kit.

In a preferred embodiment of the kit, the standard (1) comprises an indicative amount of at least one biomarker selected from the first group of said biomarkers and/or the at least one standard (2) comprises an indicative amount of at least one biomarker selected from the second group of said biomarkers.

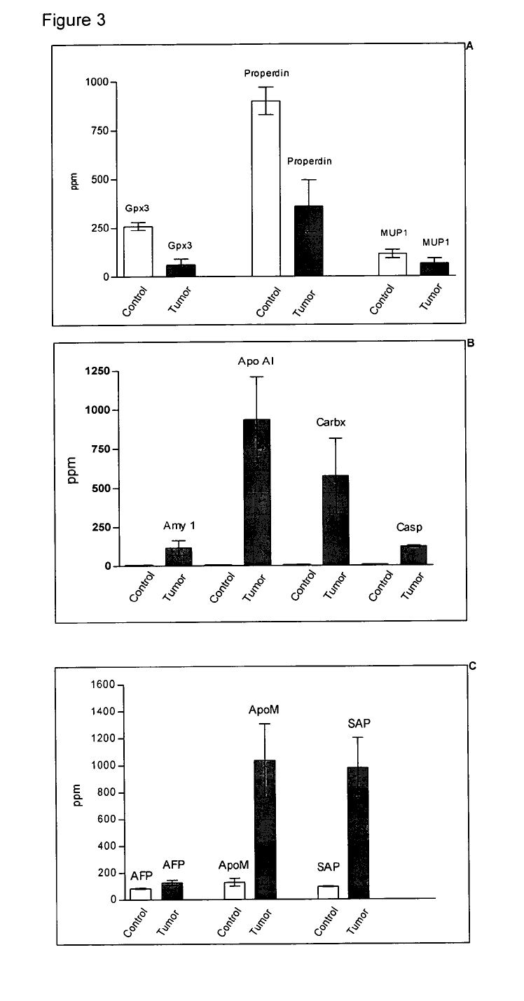

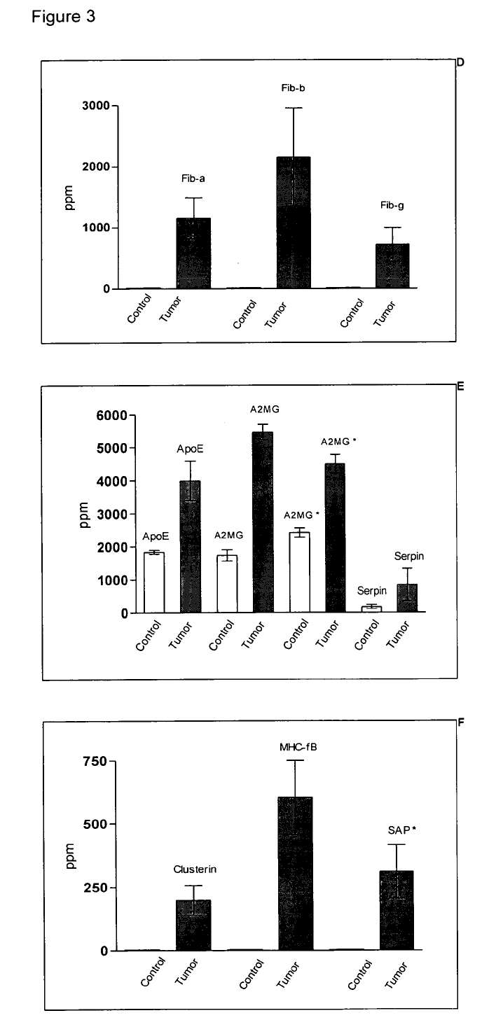

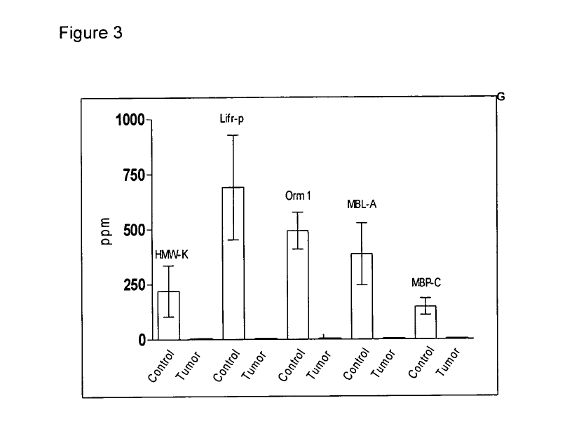

In another preferred embodiment, the kit comprises a mixture of the at least one standard (1) and the at least one standard (2), in particular a composition according to the invention comprising an effective amount of at least one FIG. 3 from the first group of said biomarkers and an effective amount of at least one biomarker selected from the second group of said biomarkers, wherein the set of biomarkers according to combination (a) or combination (b), as described herein, is particularly preferred.

In yet another preferred embodiment, the kit according to the invention further comprises a lysis buffer, wherein the lysis buffer comprises (a) at least one buffer component, (b) at least one chaotrope, (c) at least one detergens, (d) at least one reducing agent (e) at least one carrier ampholyte, and (f) at least one ribonuclease,

Preferably, the lysis buffer is an aqueous solution of (a) at least one buffer compound selected from the group consisting of Tris and HEPES, (b) at least one chaotrope selected from the group consisting of urea and thiourea, (c) at least one detergens selected from the group consisting of CHAPS and SDS, (d) at least one reducing agent selected from the group consisting of DTT and TCEP, (e) at least one carrier ampholyte selected from the group consisting of biolyte 5-7 and biolyte 3-10, and (f) at least one ribonuclease selected from the group consisting of endonuclease and exonuclease, wherein an aqueous solution of (a) Tris; (b) urea and thiourea, (c) CHAPS, (d) DTT, (e) biolyte 3-10, and (f) endonuclease, is particularly preferred.

In one preferred embodiment, the kit according to the invention further comprises at least one antibody specific for a biomarker selected from the first group of said biomarkers and/or at least one antibody specific for a biomarker selected from the second group of said biomarkers, and reagents effective to detect said biomarker(s) in a serum sample, such as buffers for dissolving or equilibrating the standard (1) and/or the standard (2), or an enzyme substrate for imaging enzyme labels may be. In particular, a kit is preferred, comprising at least one antibody specific for a biomarker selected from the group consisting of Amy 1, Apo Al, Carbx, Casp, Fib-a, Fib-b, Fib-g, Clusterin, MHC-fB, SAP isoform and/or at least one antibody specific for a biomarker selected from the group consisting of HMW-K, Lifr-p, Orm 1, MBL-A, MBP-C.

More particular, it is preferred, if the at least one antibody is polyclonal, thus allowing a further enhancement of the system sensitivity.

Advantageously, the kit further comprises at least one labelled secondary antibody specific for the at least one antibody, thus allowing a fast screening of the binding of the at least one antibody to the at least one biomarker, in particular if the at least one biomarker or the digest thereof is immobilized to a solid phase support, such as nitrocellulose may be.

In a further aspect, the invention provides a method of qualifying the EGFR kinase activity in a subject, comprising determining in a body fluid sample of a subject suffering from or being susceptible to cancer at least one biomarker selected from the first group of said biomarkers and/or at least one biomarker selected from the second group of said biomarkers, wherein the body fluid level of the at least one biomarker of said first group being significantly higher and/or the body fluid level of the at least one biomarker of said second group being significantly lower than the level of said biomarker(s) in the body fluid of subjects without cancer, in particular without cancer associated with increased activity of EGFR, is indicative of induced EGFR kinase activity in the subject.

In particular, it is preferred, if the method comprises determining at least one biomarker selected from the first group of said biomarkers and at least one biomarker selected from the second group of said biomarkers, wherein the body fluid level of the at least one biomarker of said first group being significantly higher and the body fluid level of the at least one biomarker of said second group being significantly lower than the level of said biomarkers in the body fluid of subjects without cancer, in particular without cancer associated with increased activity of EGFR, is indicative of induced EGFR kinase activity in the subject, preferably if a combination of a biomarker selected from the group consisting of Amy 1, Apo Al, Carbx, Casp, Fib-a, Fib-b, Fib-g, Clusterin, MHC-fB, SAP isoform and a biomarker selected from the group consisting of HMW-K, Lifr-p, Orm 1, MBL-A, MBP-C

or a combination of
a biomarker selected from the group consisting of AFP, ApoE, ApoM and a biomarker selected from the group consisting of Gpx3, A2MG, A2MG isoform, SAP is determined.

Preferably, the method according to the invention is carried out for predicting the response of a cancer patient to a method of treating cancer comprising administering an EGFR kinase modulator, wherein the body fluid level of the at least one biomarker of said first group being significantly higher and/or the body fluid level of the at least one biomarker of said second group being significantly lower than the level of said biomarker(s) in the body fluid of subjects without cancer, in particular without cancer associated with increased activity of EGFR, is indicative that the subject will respond therapeutically to a method of treating cancer comprising administering an EGFR kinase modulator.

In one embodiment, the method is implemented for monitoring the therapeutically response of a cancer patient to a method of treating cancer comprising administering an EGFR kinase modulator, wherein the body fluid level of the at least one biomarker of said first group before and after the treatment and/or the body fluid level of the at least one biomarker of said second group before and after the treatment is determined, and a significant decrease of said body fluid level(s) of the at least one biomarker of said first group and/or a significant increase of said body fluid level(s) of the at least one biomarker of said second group after the treatment is indicative that the cancer patient therapeutically responds to the administration of the EGFR kinase modulator.

In a preferred embodiment, the method is implemented by performing an immunoassay, such as an enzyme immunoassay (EIA), a radio immunoassay (RIA) or a fluorescence immunoassay (FIA) may be, in particular by using the kit according to the invention and/or by performing a western blot. Preferably, at least one antibody specific for a biomarker selected from the group consisting of Amy 1, Apo Al, Carbx, Casp, Fib-a, Fib-b, Fib-g, Clusterin, MHC-fB, SAP isoform and/or at least one antibody specific for a biomarker selected from the group consisting of HMW-K, Lifr-p, Orm 1, MBL-A, MBP-C is used for the immunoassay and/or reagents effective to detect said biomarker(s) in a serum sample, such as a blocking buffer for reducing unspecific antibody binding or an enzyme substrate for imaging enzyme labelled antibodies may be, is used for the immunoassay.

In another preferred embodiment, the method is implemented by performing a peptide mass fingerprinting, in particular by using the kit described herein.

Within the context of peptide mass fingerprinting, the method preferably comprises
the steps of
   isolating a serum sample from a blood sample of a subject suffering from or being susceptible to cancer,
   adding lysis buffer to the serum sample;
   separating the proteins of the lysed serum sample by 2-DE gel electrophoresis;
   excising from the gel at least one sample containing a protein of interest;
   adding digesting buffer to the at least one excised sample, and
   determining the amount of the at least one protein of interest by analyzing the at least one digest mixture by mass spectrometry.

In one embodiment of the method, the subject is a human patient or a non-human transgenic animal, in particular suffering from or being susceptible to cancer, more particular suffering from or being susceptible to cancer of the liver, lung, breast, colon, prostate, bladder, head and neck, ovary or brain, such as a transgenic mouse, in particular a mouse whose genome comprises a non natural IgEGF sequence, may be.

In another embodiment of the method, the serum sample is isolated by centrifuging the blood sample;

In yet another embodiment of the method, the 2-DE is performed by using two different pH gradients, preferably by using the pH gradients 3-10 and 4-7.

In a further embodiment of the method, the lysis buffer comprises (a) at least one buffer component, (b) at least one chaotrope, (c) at least one detergens, (d) at least one reducing agent (e) at least one carrier ampholyte, and (f) at least one ribonuclease. Preferably, the lysis buffer used is an aqueous solution of (a) at least one buffer compound selected from the group consisting of Tris and HEPES, (b) at least one chaotrope selected from the group consisting of urea and thiourea, (c) at least one detergens selected from the group consisting of CHAPS and SDS, (d) at least one reducing agent selected from the group consisting of DTT and TCEP, (e) at least one carrier ampholyte selected from the group consisting of biolyte 5-7 and biolyte 3-10, and (f) at least one ribonuclease selected from the group consisting of endonuclease and exonuclease, wherein an aqueous solution of (a) Tris; (b) urea and thiourea, (c) CHAPS, (d) DTT, (e) biolyte 3-10, and (f) endonuclease, is particularly preferred. the lysis buffer comprises (a) at least one buffer component, (b) at least one chaotrope, (c) at least one detergens, (d) at least one reducing agent (e) at least one carrier ampholyte, (f) at least one ribonuclease is particularly preferred.

In yet a further embodiment of the method, the protein of interest is a biomarker selected from the first group of said biomarkers or is a biomarker selected from the second group of said biomarkers, in particular is selected from the first group consisting of Amy 1, Apo Al, Carbx, Casp, Fib-a, Fib-b, Fib-g, Clusterin, MHC-fB, SAP isoform or from the second group consisting of HMW-K, Lifr-p, Orm 1, MBL-A, MBP-C, or more preferably is selected from the first group consisting of AFP, ApoE, ApoM or from the second group consisting of Gpx3, A2MG, A2MG isoform, SAP.

In another embodiment of the method the digesting buffer comprises a bicarbonate compound and a protease, wherein the digesting buffer preferably is an aqueous solution of at least one bicarbonate compound selected from the group consisting of ammonium bicarbonate and sodium bicarbonate and of at least one serine protease, in particular selected from the group consisting of trypsin, chymotrypsin and elastase, or, in particular preferred, the digesting buffer is an aqueous solution of ammonium bicarbonate and trypsin.

In yet another embodiment of the method, the mass spectrometry is selected from the group consisting of MALDI-TOF and ESI-TOF, preferably the mass spectrometry is performed by MALDI-TOF.

In a further embodiment of the method, a tandem mass spectrometer is used for the peptide mass fingerprinting, wherein a MALDI-TOF/TOF spectrometry is particularly preferred for putting the method into practice.

In yet a further embodiment of the method, a matrix is used for the mass spectrometry selected from the group consisting of 3,5-dimethoxy-4-hydroxycinnamic acid, α-cyano-4-hydroxycinnamic acid and 2,5-dihydroxybenzoic acid, wherein α-cyano-4-hydroxycinnamic acid is particularly preferred as the matrix.

In another preferred embodiment of the method, the serum sample is calibrated or the serum samples are equilibrated to a predefined protein concentration by adding the lysis buffer, thus allowing an easy adaptation of the system to different purposes.

In particular, it is preferred, if the method further comprises the steps of
determining the protein concentration of the serum sample, in particular by the Bradford method; or
freezing and thawing the serum sample before the lysis buffer is added; and/or
staining the gel after the 2-DE, in particular by using coomassie blue; and/or
destaining the exised sample; or
shrinking, in particular by adding acetonitrile, and drying of the excised sample before the digesting buffer is added; or
using a peptide calibration standard for the mass spectrometry,
   wherein preferably a combination of said steps, in particular two of said steps, more preferably three of said steps, in particular four or five of said steps, most preferably all of said steps are implemented.

Yet another aspect of the invention concerns a procedure to screen for and to identify drugs against cancer associated with an increased EGFR kinase activity, wherein the procedure comprises determining in a body fluid sample of a transgenic cancer mouse being treated with a compound to be tested, in particular of a mouse whose genome comprises a non natural IgEGF sequence, at least one biomarker selected from the first group of said biomarkers and/or at least one biomarker selected from the second group of said biomarkers, and wherein the body fluid level of the at least one biomarker of said first group being significantly lower and/or the body fluid level of the at least one biomarker of said second group being significantly higher than the level of said biomarker(s) in the body fluid of an untreated transgenic cancer mouse is indicative of the therapeutic effect of said compound as an EGFR kinase modulator.

In a preferred embodiment, the procedure is implemented by using the method according the invention, in particular by using the method comprising an immunoassay or a peptide mass fingerprinting as described herein.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Epidermal growth factor (EGF) is an important mitogen for hepatocytes. Its targeted overexpression induced hepatocellular carcinomas (HCC), as recently reported by us (Borlak et al. 2005). Early detection of disease is essential for successful therapy and overall survival. In particular, the efforts in identifying serum biomarkers of liver cancer in a transgenic disease model that mimics effectively the consequence of exaggerated EGF signalling are described. A reference 2-DE map of mouse serum proteins is reported. About 180 proteins were detected per gel and 130 proteins were identified by 2-DE-MALDI-MS analysis. Serum proteins of healthy non-transgenic and HCC tumor bearing mice were compared and 25 regulated proteins were identified, of which n=7 reached statistical significance (p<0.05). Furthermore, several fragments of fibrinogens and of the alpha-2-macroglobulin were identified to be disease associated. Also immunoglobulins were found to be repressed or absent in serum samples of tumor bearing mice, and this included, amongst others, the Ig K and L class. In contrast, amyloid component P and apolipoprotein M were highly significantly increased by 10- and 8-fold, respectively in serum samples of HCC-mice. Overall, the findings provide a rationale for further clinical evaluation of the herein identified biomarker candidates.

In the following, the findings with an EGF transgenic disease model of liver cancer are reported. This is an important growth factor mitogen for hepatocytes. Its targeted overexpression promoted hepatocellular carcinogenesis as recently reported by us.⁸ In general, the EGF gene codes for a 53 amino acid protein to stimulate proliferation of epidermal cells and a variety of other cell types through binding to the EGF receptor. This single-pass transmembrane receptor functions as a tyrosine kinase. Once activated, the EGFR becomes autophosphorylated to initiate signalling through tyrosine phosphorylation of other proteins.⁹ A total of four different EGF receptors (Her1, Her2, Her3, Her4) have been identified so far. Specifically, ligand binding induces either homo- and/or heterodimerisation. It is of considerable importance that EGFR connects to other signalling cascades as well, notably the MAP kinase pathway, to ultimately cause phosphorylation of transcription factors such as c-Fos, c-Jun and ELK-1, thereby fostering cell proliferation.⁹ EGFR is over expressed in a number of solid tumours and the expression level correlates well with tumour progression, resistance to chemotherapy and survival. Consequently, EGFR is an obvious target for the rational design of novel anticancer agents, i.e. inhibitors of the receptor kinase activity and/or antagonistic antibodies.¹⁰

Previously, targeted overexpression of a secretable form of EGF (IgEGF) has been reported to result in multiple highly malignant HCCs, with 100% fatalities around 7-8 months after birth.¹¹ This transgenic mouse line mimics effectively the consequence of an altered EGF and/or TNFα signalling. Recently, other mice models were successfully established to study the tumor biology of HCC in non-viral- and viral disease¹²⁻¹³. Through application of gene chip analysis it was possible to identify networks of EGF-regualted genes at various stages of tumour development.⁸ Here, the efforts to examine serum proteins of non-transgenic healthy and EGF overexpressing transgenic mice are reported. A reference 2-DE map of mouse serum proteins, consisting of more than 130 proteins, was created. After 2-DE analysis and gel image matching, 25 serum proteins were identified by peptide mass fingerprinting as being disease associated. These proteins are involved in a variety of cellular and metabolic pathways, amongst them: the glutathione peroxidase 3 (Gpx3) and the serum amyloid component P (SAP), their expression being up-regulated by up to 10-fold in tumour bearing mice.

Overall, this study aimed at identifying disease associated serum proteins in an EGF liver cancer disease model.

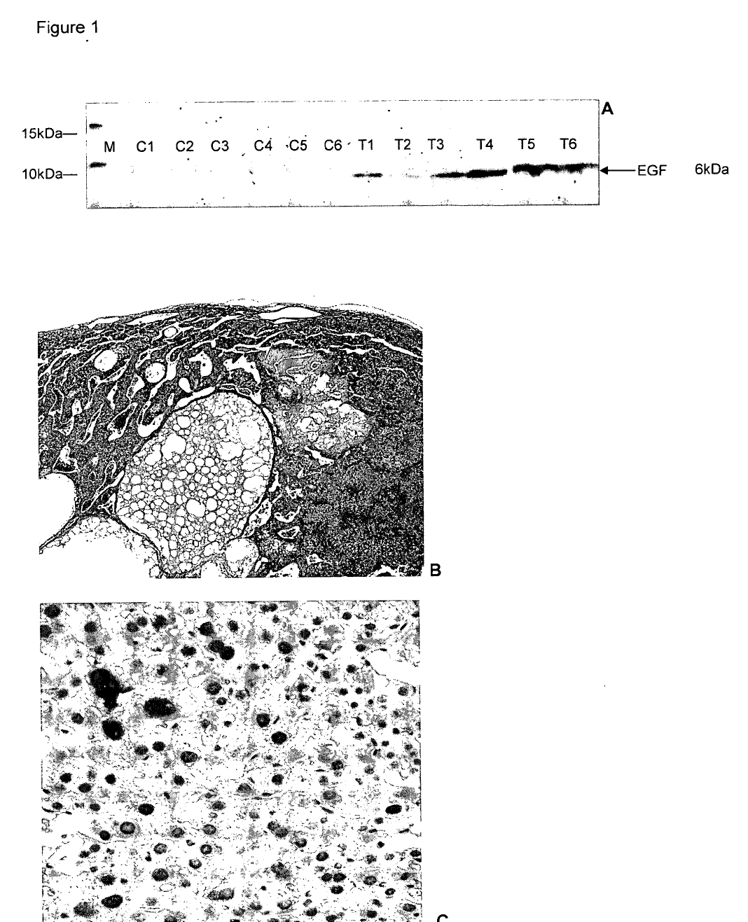

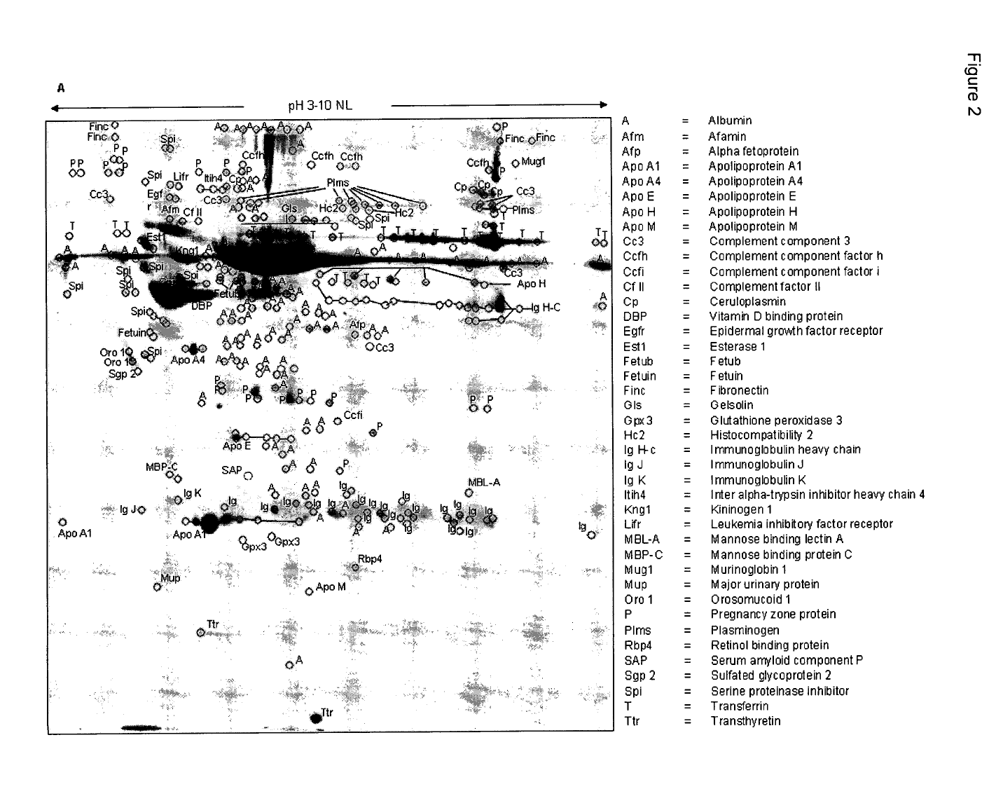

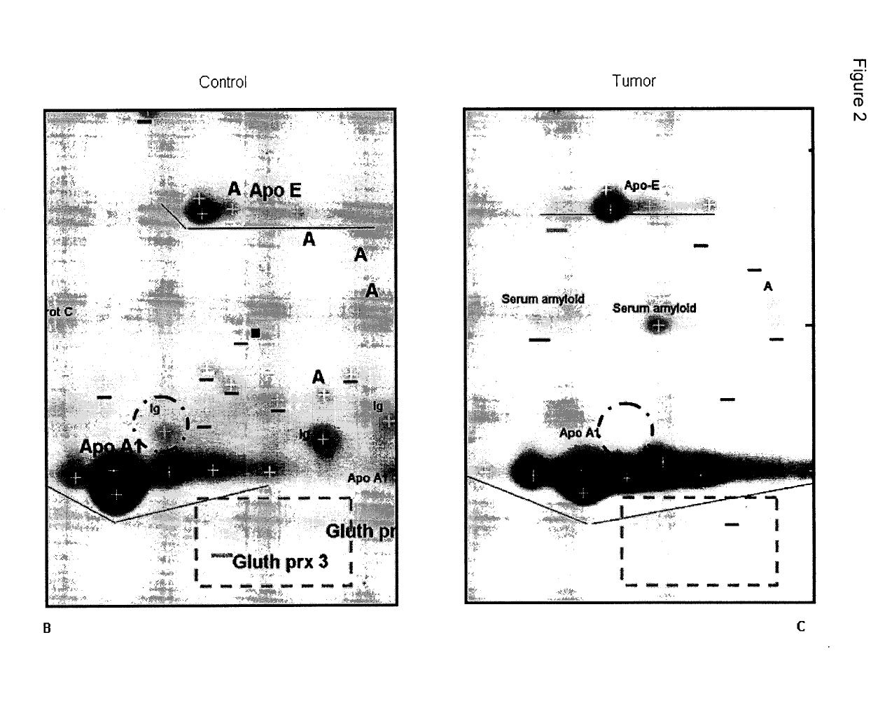

The serum proteome of healthy and HCC tumor bearing mice was investigated. As depicted in FIG. 1A, transgenic mice overexpressed EGF by approximately 26-fold. Furthermore, tumors were confirmed by histopathology, of which an example is given in FIG. 1B. Essentially, highly differentiated tumors were observed which were less differentiated at advanced stages of disease (see also Borlak et al., 2005 for further details).⁸ A total of 14 2-DE gels (n=5 independent serum control and n=9 independent serum of HCC mice) were processed for MALDI-TOF-MS analysis (see supplementary material 1, FIG. 5). This enabled the built up of a reference 2-D map of mouse serum proteins consisting of 130 proteins (FIG. 2A). 25 proteins were found to be differentially regulated of which n=7 were statistically significant (Table 1, FIG. 2B, 2C). Albumin is the most abundant serum protein; its quantification of the 2-DE gels gave a relative volume of approximately 34% (see supplementary material 2, FIG. 6). Other high abundance proteins include alpha-2-macroglobulin with a relative volume of 10%, transferrin (7%), apolipoprotein A1 (4%), plasminogen (4%) and several immunoglobulins (Igs) such as H—C, K and L (4%). Less abundant proteins were apolipoprotein E, apolipoprotein H, fibronectin, gelsolin, kininogen and ceruloplasmin. Several spots were identified at low molecular weights as albumin or fragments of it (FIG. 2 A) and more than 8 spots as alpha-2-macroglobulin (A2MG) (fragments) with mass ranges of 37-40 kDa (A2mG has a Mr of ˜165 kDa). At least 2 of these fragments were up-regulated in serum of tumour bearing mice and were increased by 3-fold (see table 1, FIG. 3E, supplementary material 8, FIG. 12). Furthermore, two isoforms were identified with identical pl but different Mr as well.

Additionally, the apolipoproteins Apo A-I, Apo E, Apo H, ApoM and ApoJ were identified. 6 different spots in the serum proteome maps were observed, which were identified by MALDI-TOF as Apo A-I (see supplementary material 3, FIG. 7) and at least 5 spots of apo H, also known as beta-2-glycoprotein I (see supplementary material 4, FIG. 8).

After in gel digestion, approximately 2500 spots were selected for MALDI-TOF-PMF analysis. In the supplementary table 1 accession number, protein name, theoretical pl and Mr together with Mascot score, identified peptides and sequence coverage are given. Furthermore, biological function, frequency of identification and protein expression in serum of tumor and/or healthy mice are summarized as well (see supplementary table 1). Among the 130 annotated proteins, 25 were differentially expressed (Table 1), of which 7 were up-regulated (apolipoprotein E, alpha-2-macroglobulin, alpha-2-macroglobulin*, alfafetoprotein, apolipoprotein M, serum amyloid component P, serine proteinase inhibitor) (FIG. 3C, 3E), where as 3 proteins were down-regulated (glutathione peroxidase 3, properdin, major urinary protein 1) (FIG. 3A). Moreover, 10 proteins were found in serum of tumor bearing mice only (amylase 1, apo A-I, carboxylesterase, caspase, clusterin, fibrinogen-α, fibrinogen-β, fibrinogen-γ, major histocompatibility complex factor B, serum amyloid component P*), but 5 proteins were exclusively identified in serum sample of healthy control animals (mannose binding lectin A, orosomucoid 1, HMW-kininogen II, leukemia inhibitory factor receptor, mannose binding protein-C) (FIG. 3G).

In tumor bearing mice, most of the up-regulated serum proteins are apolipoproteins. Among them, apoM was up-regulated by 8-fold (FIG. 3C, 4B, supplementary material 6, FIG. 10). ApoM is seen as an important biomarker candidate and its significance is discussed later on. Furthermore, apolipoproteins are closely associated with amyloid fibrillogenesis. Indeed, serum amyloid A, apolipoprotein (apo) All and apo Al are deposited as biochemically distinct forms of amyloid.¹⁴ In serum samples of HCC bearing mice, the serum amyloid component P (SAP) was strongly increased. This protein belongs to the group of acute-phase reactants (APRs) (FIG. 3C, 4D). Here SAP is evidenced to be highly disease associated in HCC and a putative isoform (SAP*) in sera of tumour bearing mice is identified (see table 1, supplementary material 7) with a lower theoretical pl value.

In addition, several isoforms of alpha-2-macroglobulin were found (see FIG. 2A, supplementary material 8, FIG. 12) to be induced in HCC. To the best of our knowledge, the overexpression of the newly identified fragments have not been reported for HCC so far. In general, the serum concentration of A2MG is about 2 g/l in adults but displays little variation with age or in acute and chronic disease. It therefore may qualify as a bonafide candidate serum biomarker for HCC. Further, decreased concentration of proteins of the acute phase response in serum of HCC mice were observed. This group of proteins, designated as negative acute phase proteins, included major urinary protein 1 (MUP1), glutathione peroxidase 3 (Gpx3), properdin, and several immunoglobulins (Igs). (see table 1, FIG. 3A). Specifically, plasma glutathione peroxidase 3 (Gpx3), a glycosylated protein, was repressed when serum samples from healthy non-transgenic and tumor bearing mice were compared. In 2D gels two different spots are visible e.g. Gpx3* and Gpx3 while only one isoform was repressed in serum samples of tumour bearing mice (Gpx3) (see FIG. 3A, supplementary material 9, FIG. 13).

In the present study, also cancer-related fibrinogen deposition in serum of HCC bearing mice is identified (see table 1, FIG. 3D, supplementary material 10, FIG. 14). Plasmin-generated fragments of fibrinogen alpha, beta and gamma (Fga, Fgb, Fgg) were already observed in various types of solid tumor types. In fact, elevated levels of Fgg and acute phase proteins were reported for plasma proteins of prostate-, lung-, and breast-cancer patients.¹⁵⁻¹⁶

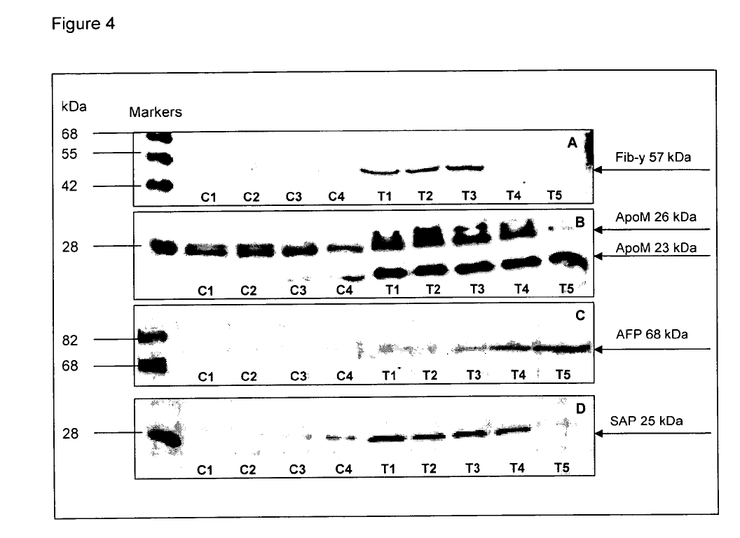

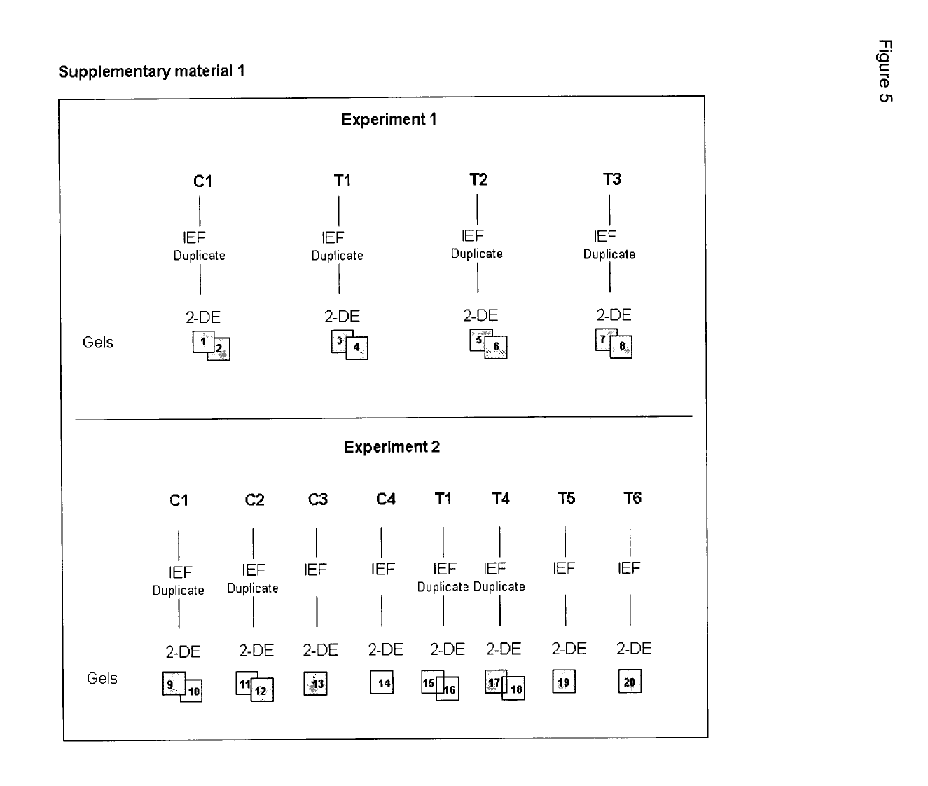

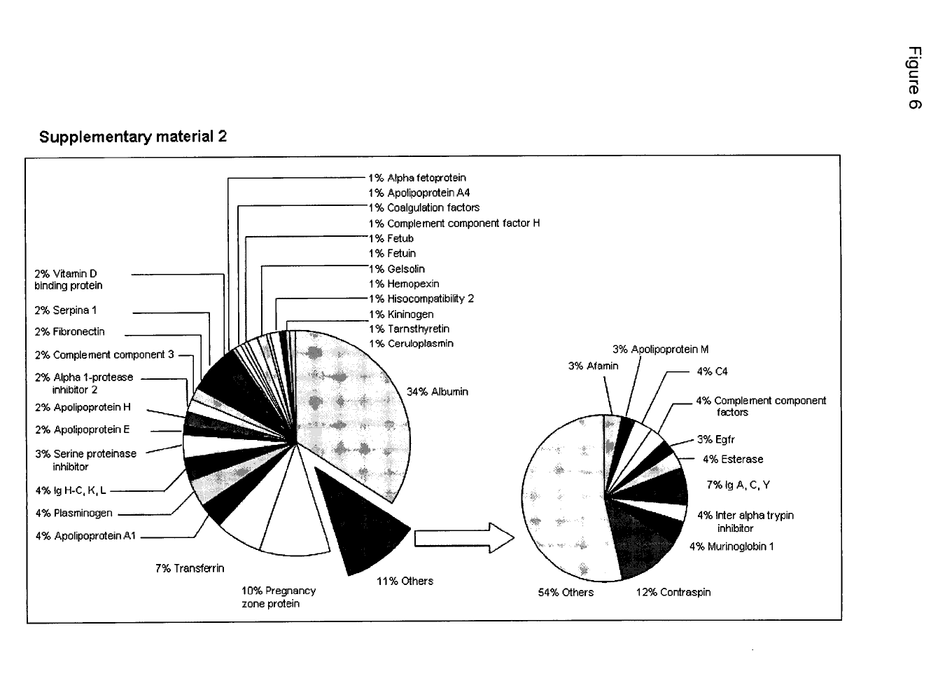

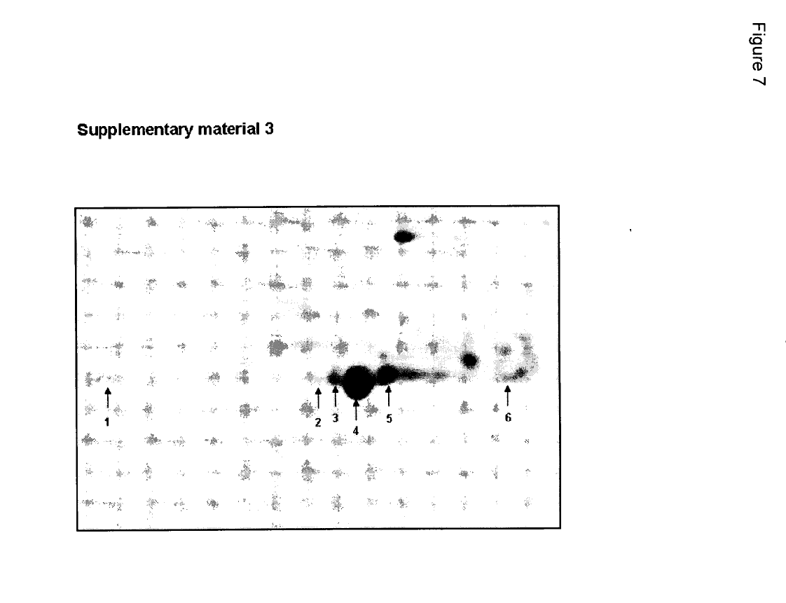

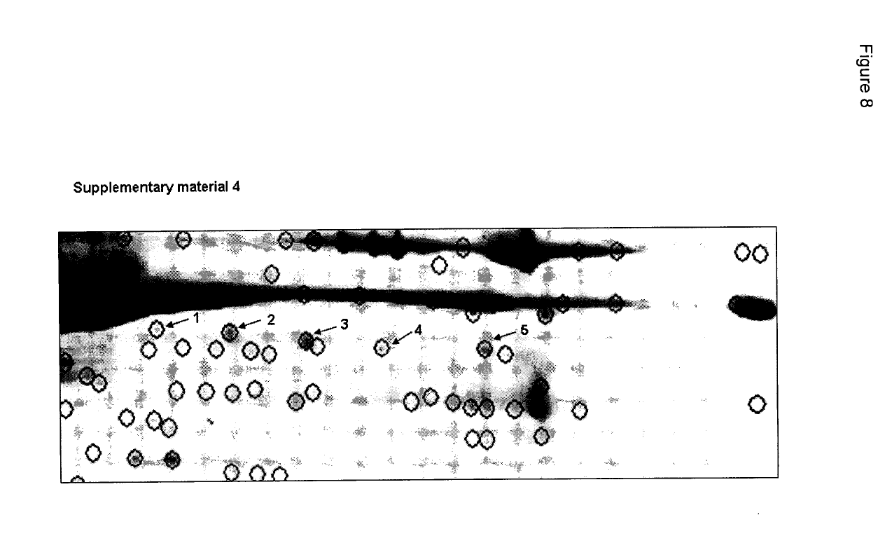

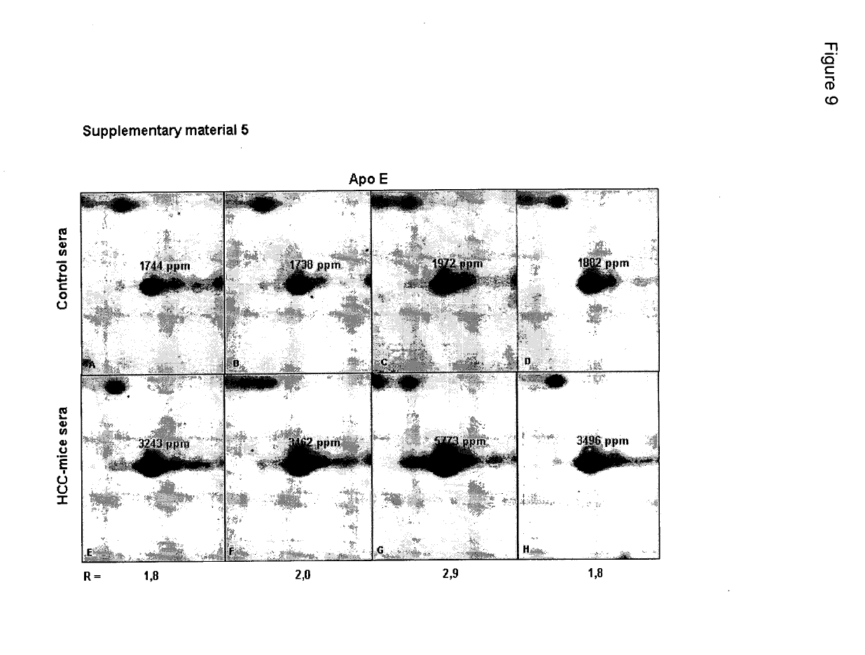

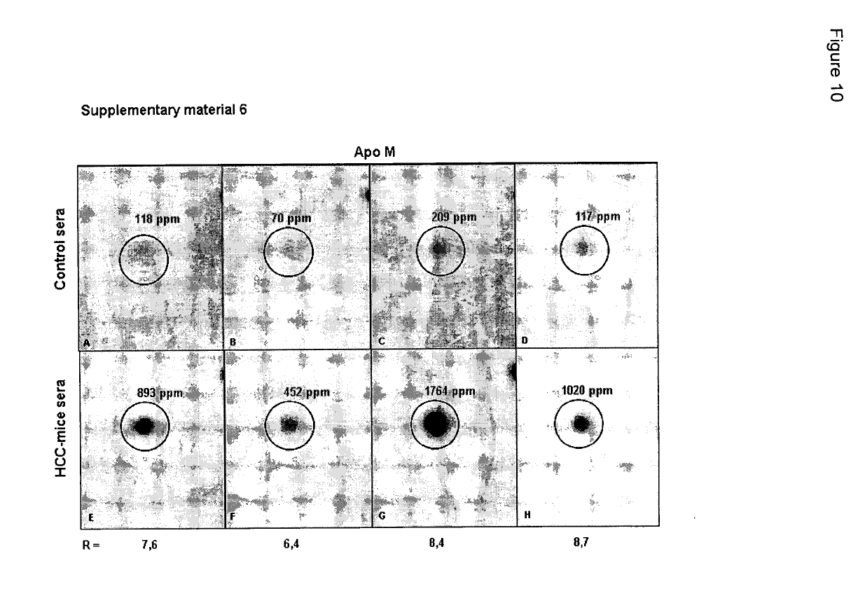

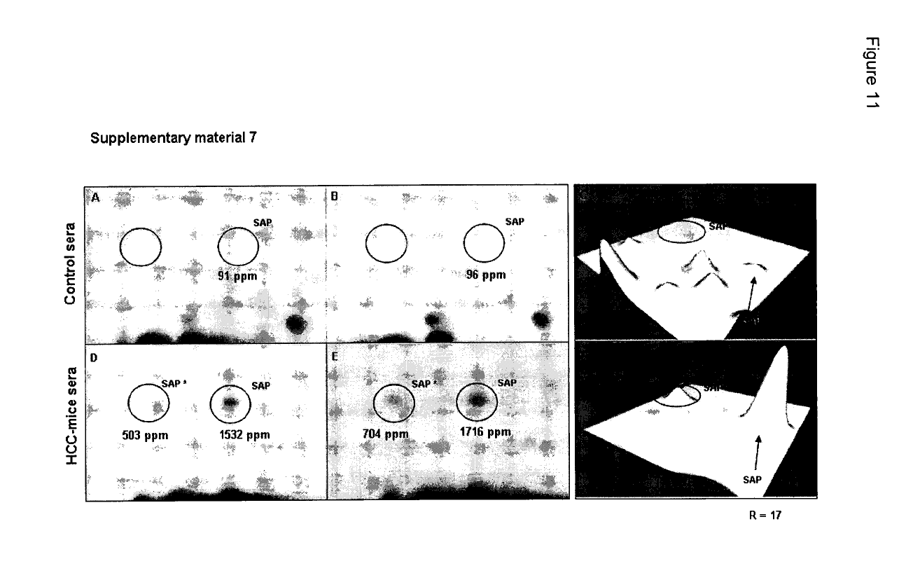

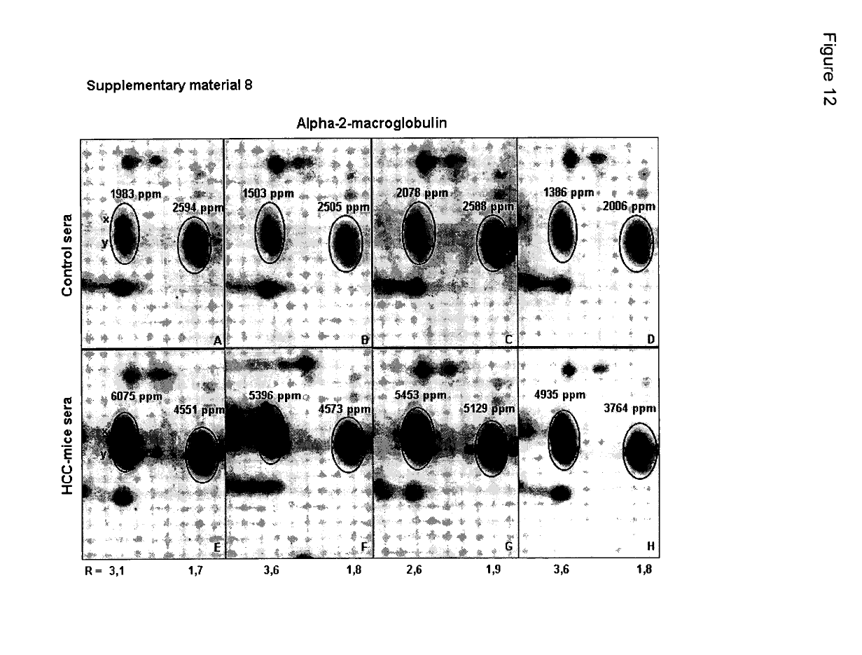

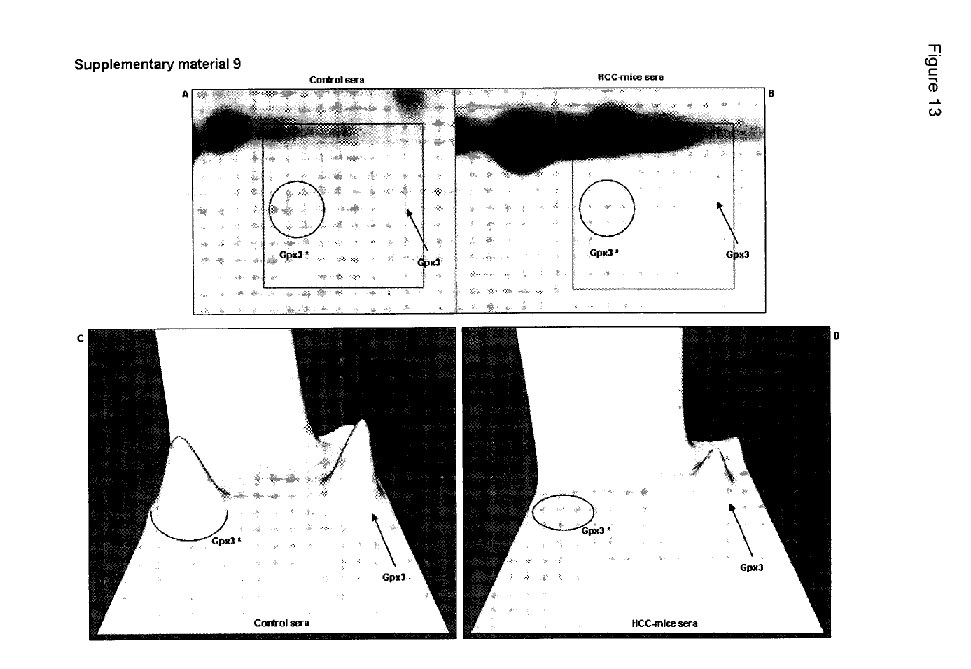

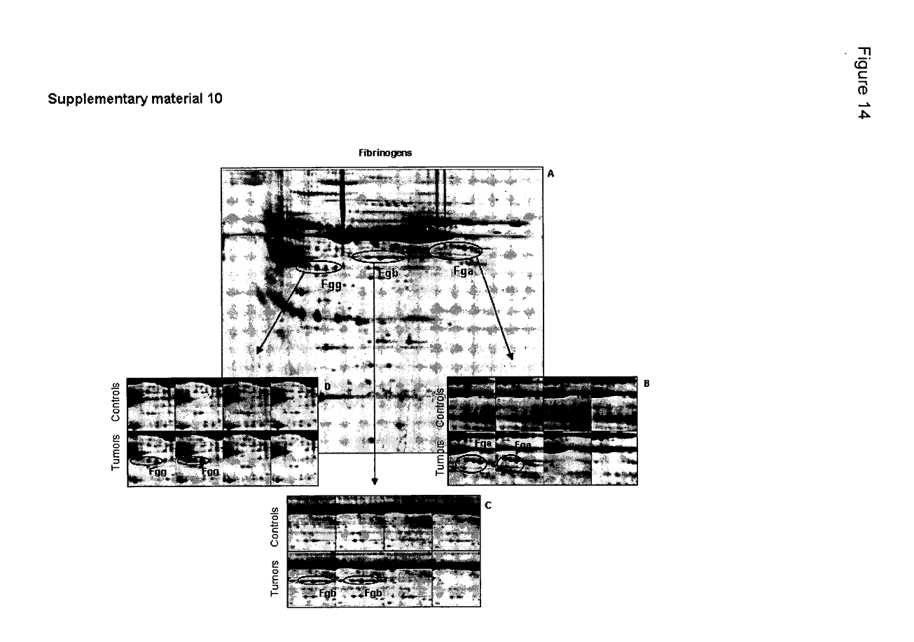

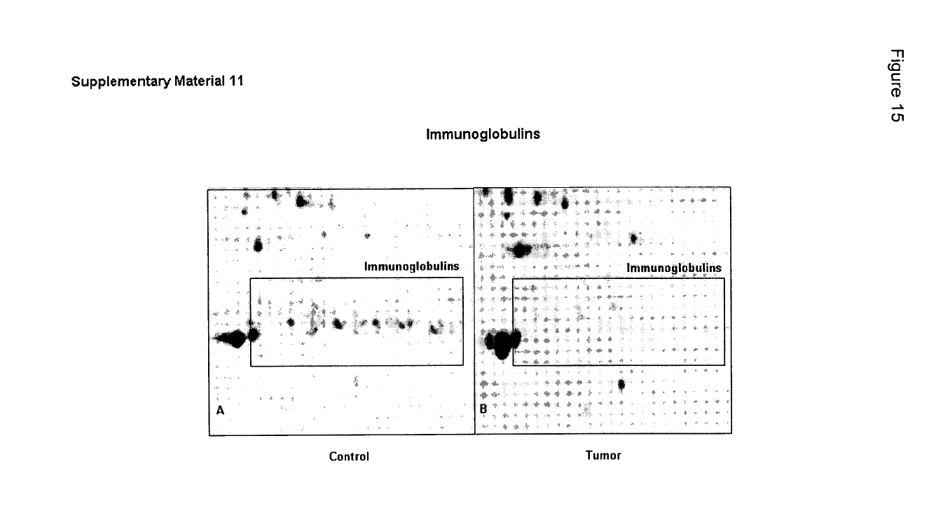

As of today overexpression of FGG was reported for two hepatoma cell lines, e.g. SMMC-7721 and HepG2.¹⁷ Up-regulation of Fgg in EGF induced HCC is now evidenced, as well. Specifically, not all tumor samples expressed the three fibrinogens (see FIG. 4A, supplementary material 10, FIG. 14), but this agrees well with the findings of Gerner et al., who reported Fgg to be present in 58% of the cases (14/24 analyzed plasma cancer samples.¹⁵⁻¹⁶ Elevated plasma levels of Fgg, as observed in serum of EGF transgenic mice may be viewed as indicators for tumor-associated fibrin deposition and fibrinolysis. Fibrinolysis is mediated mainly via the plasma protease plasmin, which exerts pleiotropic effects.¹⁸⁻²⁰

Furthermore, the complement system is known to contain at least 30 different proteins, which are primarily formed in the liver and circulate in their inactive form. These proteins, when activated, produce various complexes that play a major role in the natural defense mechanisms of the human body. Several proteins of the complement system were identified to be regulated. This included the mannose binding lectine A and the mannose binding protein C (present in serum of healthy non-transgenic mice only), and the major histocompatibility complex factor B (MHC-fB) which was found to be up regulated. MHC-fB was identified in healthy and HCC serum samples, but an activated form/fragment of this protein was found in serum of HCC mice only in the range of Mw 65-68 kDa (see table 1, FIG. 3F). These fragments could be the result of proteolitic processes in tumours.

Also, orosomucoid 1, also known as alpha 1-acid glycoprotein, was identified in sera of health non tumor bearing animals (see table 1, FIG. 3G). This protein is synthesised by the liver, and functions in modulating the activity of the immune system during the acute-phase reaction. Several studies report an up regulation of this protein in patients with HCC and suggest orosomucoid as a useful marker for discriminating the stage of inflammatory reactions.²¹⁻²³ Finally, an up regulation of the oncofetal protein Afp was observed in sera of HCC-mice (FIG. 3C, 4C). Indeed, Afp is routinely assayed for liver damage and its malignancies.

To confirm results by different means, 4 proteins regulated in all gels were selected for Western Blot analysis. A total of n=5 serum samples of tumor bearing mice were probed for Fib-γ, ApoM, Afp (FIG. 4 A,B,C), where as n=4 serum samples was used for SAP (FIG. 4 D). Fib-γ was expressed only in 3 out of the 5 serum samples analyzed.

Serum biomarkers of HCC were searched for and a serum proteome map of EGF induced HCC is reported. Comparison between sera of healthy and tumour animals revealed significant differences in expression of several proteins. A total of 25 proteins was identified as differentially expressed. Specifically, proteins of the acute phase response were found to be down regulated. This group of proteins included mannose binding lectin a (MBL-A), major urinary protein 1 (MUP 1), orosomucoid 1 (Orm1), glutathione peroxidase 3 (Gpx3) and several immunoglobulins (Igs) (see table 1, supplementary material 11). In contrast, Apo E was up regulated in serum of HCC mice. Notably, Yokoyama and coworkers observed increased expression of apo E in 88% of HCC tumor tissue without an increase of apo E gene expression and/or elevated serum level.¹⁹ These results do not agree with the findings as serum levels of apo E were increased by 2-fold (FIG. 3E and supplementary material 5, FIG. 9). An overexpression of ApoE has already been observed in brain²⁴, breast²⁵, ovarian²⁶, prostate²⁷ and HCC tumor tissues.²⁸

Another up-regulated apolipoprotein was apoM. This protein is mainly associated with high-density lipoprotein (HDL) in human plasma, and a small proportion in triglyceride-rich (TGRLP) and low-density lipoproteins (LDL). During embryogenesis, apo M is over expressed in livers of 3-5 month-old human embryos and continues to be strongly expressed throughout embryogenesis, but thereafter returns to much lower levels. Apo M is found as an important biomarker candidate. Furthermore, there is evidence for platelet-activating factor (PAF) to be involved in the up-regulation of apo M in HepG2 cells, but Luo and coworkers reported decreased apo M mRNA transcript levels in HepG2 cells in response to epidermal growth factor (EGF) treatment.²⁹⁻³⁰ It is of considerable importance that apo M gene expression may also be regulated by hepatocyte nuclear factor-1α (HNF-1α), which was found to be repressed in tumor tissue of EGF transgenic mice (results of Western Blot not shown).³¹ Furthermore, the apo M gene is located within the histocompatibility complex III (HMC-III) region of chromosome 6 and many genes in this region code for immune response.³⁰ Whether apo M is co-regulated by the host defense system requires additional research.

In general, apolipoproteins are important in maintaining the structural integrity of lipoprotein particles thereby facilitating the solubilisation of lipids. Additionally, they play a crucialrole in lipoprotein receptor recognition and regulation of lipoprotein metabolism. In humans, about 60% of the protein content in high-density lipoprotein (HDL) is represented by apo A-I and about 20% by apo A-II. Other apolipoproteins include apo A-IV, apo C, apo D and apo E. Most of these proteins are expressed as different isoforms. Recently, it was suggested that apo A-I exists in six isoforms, 4 of them displaying differences in glycosylation pattern.³²

Likewise, apo-H displays genetic polymorphism, with three alleles, namely APOH*1, APOH*2, APOH*3, at a single locus on chromosome 17.³³ This protein has been identified as a structural component of chylomicrons, very low-density lipoproteins (VLDL), low-density lipoproteins (LDL) and HDL. In human plasma, 35% of apo-His associated with chylomicrons.³⁴ Various properties have been ascribed to this protein, e.g. function as an antigen of antiphospholipid antibodies, or acute-phase reactant and may have some involvement in the HBV infection of hepatocytes as well.³⁵⁻³⁶

Our study identified A2MG to be strongly upregulated in HCC. This protein is an abundant plasma protein produced predominantly by the mammalian liver. A2MG is a member of the protease inhibitor 139 (A2M) family and is able to inhibit all four classes of proteinases by a unique ‘trapping’ mechanism located in a ‘bait region’, which contains specific cleavage sites for different proteinases. When a proteinase cleaves the bait region, a conformational change of the protein is induced, which then traps the proteinase. The entrapped enzyme remains still active against low molecular weight substrates, but is poorly accessible for reaction with high molecular weight substrates. Mouse A2MG has a Mr of ˜165 kDa; notably, more than 8 spots as A2MG fragments with mass ranges of 37-40 kDa were identified. At least 2 of these fragments were up regulated in serum of tumor bearing mice and were increased by 3-fold (see table 1, FIG. 3E, supplementary material 8, FIG. 5). Furthermore, two isoforms with identical pl but different Mw were identified as well. In breast and trophoblastic cancers, A2MG may be less useful as a tumor marker.³⁷ In contrast, serum A2MG levels in patients with ovarian carcinomas are significantly elevated.³⁸⁻³⁹

Likewise, the study also evidenced repression of glutathione peroxidase 3 (Gpx3) in HCC bearing mice. Gpx3 is one of at least 25 selenocysteine-containing protein with antioxidant properties in mammals. It is one of the five known glutathione peroxidases and is unique among members of the Gpx family as this protein is the only extracellular isoform. Gpx3 is secreted from renal proximal tubular cells and epithelial cells of the Bowman's capsule. While Gpx3 deficiency has been associated with cardiovascular disease and with renal dysfunction or infertility of males, little is known about its association with HCC.⁴³⁻⁴¹ Strikingly, plasma selenium concentration is decreased in patients with cirrhosis, as reported by Burk and coworkers, who investigated glutathione peroxidase (GSHPx-3) and selenoprotein P expression, in patients with cirrhosis and in healthy control subjects.⁴² Equally, studies on selenium availability and expression of selenoproteins (Gpx1, Gpx3, thioredoxin reductase and selenoprotein P mRNA) in mouse fibrosarcoma cells evidenced selenium to exert a statistically significant effect on Gpx3 transcript expression.⁴³ Whether selenium availability is reduced in HCC requires further studies.

Also, high proteolytic activity in sera of HCC bearing mice was observed. Recently, Chignard et al. detected protein fragments in HCC patient sera as well, identified as calreticulin and protein disulfide isomerase A3.⁴⁴ Generally, plasma proteases are capable of activating growth factors by cleavage of their inert proforms.⁴⁵ High proteolytic activity of proteases in plasma of cancer patients has been reported.¹⁶ This may also be linked to an overexpression of SAP, apo M and other serum proteins in HCC bearing mice as reported in the study. An acute phase response is observed in the majority of cancer patients⁴⁶ and could represent an adaptive response to an exaggerated proteolytic activity. In fact, during tumor growth these proteins accumulate in peripheral blood and may interfere by a direct anti-apoptotic mode on tumor necrosis factor-induced apoptosis of hepatocytes as suggested by Van Molle et al, 1997.⁴⁷⁻⁴⁸

Finally, disease associated regulation of a fragment of Factor B in serum of HCC mice was found. Factor B is a serine proteinase of the antibody-independent, alternative pathway of complement activation, an important humoral response of the host defense system against invading pathogens. In addition, fragments of the factor B exert cytokine-like activities to cause B lymphocyte proliferation and differentiation, macrophage spreading and monocyte mediated cytotoxicity. The major site of MHC-fB expression is the liver, as evidenced by allotype changes of serum MHC-fB following liver transplantation. MHC-fB is a positive acute phase reactant. It's hepatic synthesis and serum level are increased during the acute phase of the inflammatory response.⁴⁹

Overall, serum amyloid component P (SAP), apolipoprotein M (apo M), alpha-2-macroglobulin (A2MG) and fibrinogens (Fga, Fgb, Fgg) are found as beneficial candidates for HCC-diagnostics, since their serum levels were increased by 10-, 8- and 3-fold, respectively. Furthermore, fibrinogens were identified in serum samples of HCC mice only, but their expression was variable. The current available screening tests to detect early liver malignancies combine α-fetoprotein analysis and ultrasound. Even though screening for early detection of HCC has become more common, its effectiveness in disease diagnostics remains controversial.⁵⁰ There is a need to search for new, robust and specific markers for the detection of HCC at early stages of disease to allow for curative rather than palliative interventions. Since blood serum contains high concentrations of abundant proteins such as albumin, transferrin, haptoglobin and immunoglobulins, their removal may be advantageous to enable detection and identification of less abundant proteins. Unfortunately, many of the target proteins are bound to abundant proteins; thus, depletion of serum of major proteins using affinity chromatography, specific antibodies or size-exclusion filtration may also lead to removal of less abundant proteins and therefore loss of biomarkers. In conclusion, the use of 2-DE combined with MALDI-TOF-MS, analysis provided evidence for 25 differentially expressed proteins in serum of HCC bearing mice, of which n=7 reached statistical significance. Several proteins, so far unknown to be regulated in HCC, have been identified and represent beneficial biomarkers useful for implementing the invention.

### Materials

A UP 200S sonicator (Dr. Hielscher GmbH, Germany) was used to homogenize the samples. For the first dimension, immobilized pH-gradient (IPG) strips (17 cm, pH 3-10 non linear) were purchased from Bio-Rad (Hercules, Calif. USA). The focusing chamber was Protean IEF Cell (Bio-Rad). For the second dimension a Protean plus Dodeca Cell (Bio-Rad) was used.

Reagents: tris, urea, thiourea, CHAPS, dithiothreitol, bromophenol blue, glycerin, sodium dodecyl sulphate, glycin, temed, ammoniumperoxodisulphate, ammonium sulphate, ammonium bicarbonate, colloidal coomassie blue and acrylamide were purchased from Roth (Karlsruhe, Germany). Iodacetamide was from SERVA (Heidelberg, Germany). Benzonase was purchased from Novagen (Darmstadt, Germany). Ampholytes (Biolyte 3-10) were purchased from Bio-Rad (Hercules, Calif. USA).

### Animal Care

The creation of the EGF2B transgenic line was described earlier by Tönjes et al. (1995). Transgenic mice were maintained as hemizygotes in the CD2F1-(DBA/2xBalb/c) background. PCR was carried out with Platinum PCRSuperMix (InVitrogen). Annealing temperature and the number of cycles are indicated in brackets after each primer pair. The transgene was verified by PCR of DNA extracted from tail biopsies (Hogan et al., 1994) and the following forward primer (fp) and reverse primer (rp) pair was used for a transgene specific amplification: forward primer: 5′-CTAGGCCAAGGGCCTTGGGGGCTCTTGCAG-3′ (SEQ ID NO 1); reverse primer: 5′-CATGCGTATTTGTCCAGAGCTTCGATGTA-3′ (SEQ ID NO 2) (61° C., 32 cycles, 317 bp).

Animals, aged 6-8 months and of the weight of 25-33 g, were housed in Makrolon® Type III cages. Drinking water and food (V1124-000, SSNIFF, Holand) was given ad libitum. Temperature and relative humidity were 22±2° C. and 40-70% respectively. Furthermore, a 12 h day and night cycle was used. For serum protein identification, mice were sacrificed with CO₂ and blood was taken from the Vena cava. Then, blood was centrifuged (20 min., 6000 rpm, at room temperature) and serum was immediately frozen at −80° C. None of the serum samples were hemolytic.

### Sample Preparation

Five micro liters of sera from n=4/group non-transgenic control and from n=6/group tumor bearing animals were used for further 2-DE analysis. The protein concentration of serum was determined by the Bradford method. For each animal 0.5 mg of protein sample, approximately 3 μL of serum, were diluted to a final volume of 350 μL with a lysis buffer containing 40 mM tris base, 5 M urea, 2 M thiourea, 4% CHAPS, 100 mM DTT, 0.5% (v/v) biolyte 3-10 and endonuclease (6 μL/mL).

### Two-Dimensional Gel Electrophoresis-2-DE

A total of n=8 2-DE gels for control serum samples and n=12 2-DE gels for serum of tumor bearing mice were prepared as follow: IEF was performed using precast 17 cm IPG strips 3-10 NL. 0.5 mg of protein was loaded by active rehydration (12 h, 50 V). Focusing started at 250 V for 20 min. in rapid mode, 10000 V for 5 h in linear mode and 10000 V for 50000 Vh in rapid mode. Each sample was analyzed in duplicate.

After IEF, the IPG strips were either stored at −80° C. or transferred to 10 mL equilibration buffer (6 M urea, 30% w/v glycerin, 2% w/v SDS, 50 mM Tris-HCl pH 8.8) with 2% w/v DTT and 0.5% v/v bromophenol blue solution (0.25% w/v bromophenol blue, 1.5 M Tris-HCl pH 8.8, 0.4% w/v SDS) and incubated for 20 min. at room temperature. Strips were removed and incubate in equilibration buffer with 4% w/v iodoacetamide and 0.5% v/v bromophenol blue solution for further 20 min. at room temperature. Finally, strips and 10 μL SDS-PAGE molecular weight standard on filter paper were placed on top of the 20 cm×20.5 cm 12% second-dimension gel (12% v/v acrylamide/bis solution, 375 mM Tris, pH 8.8, 0.1% v/v SDS, 1/2000 TEMED, 0.05% v/v APS). Both were fixed in place with a 0.5% w/v agarose overlay. Gels were run in PROTEAN Plus Dodeca cell from Bio-Rad at 70 V for approximately 14 h, followed by 200 V until the bromophenol blue dye reached the bottom of the gel. The running buffer (25 mM Tris, 0.2 M glycin, 0.1% SDS) was cooled externally to 16° C.

Gels/proteins were fixed overnight in 30% ethanol, 2% phosphoric acid, washed 3×20 min. with 2% phosphoric acid. The gels were equilibrated with 15% ammoniumsulfate, 18% ethanol, 2% phosphoric acid for 15 min and finally stained with colloidal coomassie blue for 48 h.

### Gel Scanning and Image Analysis

After staining, gels were washed 10 min with pure water and scanned on a Molecular FX Scanner Bio-Rad at 100 μm resolution. Protein spots were imaged first automatically and then manually and analyzed using the PDQuest™ software Bio-Rad. The normalization was carried out in total density in gel mode according to the manufactures recommendations. For reproducibility, two experiments were performed, each time running 4-control and 6-tumor samples for a total of 20 gels. From them, 14 gels were chosen and used for spot excision and MS analysis. The gels with higher number of spots were selected; all spots (about 180 spots per gel) were excised for MS analysis

### MALDI-MS

A total of 2500 spots from 14 gels were excised using the spot cutter of Bio-Rad and placed into 96-well microtiter plates. Excised gel spots were washed with 20 μL of water for 10 min. and destained twice with 15 μL ammonium bicarbonate 50 mM for 5 min. first and then with 15 μL 50% ammonium bicarbonate 50 mM-50% acetonitrile for 5 min. Finally, gel particles were covered by acetonitrile until gel pieces shrunk and left dry for 10 min. Gels/proteins were digested in situ with 4 μL of ammonium bicarbonate 50 mM containing 20 ng trypsin (Sequencing Grade Modified Trypsin Promega). After 15 min. each gel peace was re-swelled with 10 μL of ammonium bicarbonate 50 mM and incubated for 4 h at 37° C. After 4 h the reaction was stopped by adding 10 μL of trifluoro acetic acid 1% containing 1.5% (w/v) n-octyl-β-D-glucopyranoside (OGP) (AppliChem). For the application of the samples, 4 μL of peptide solution were loaded on a MTP Anchor Chip Target 600/384 (Bruker Daltonics) previously prepared with a saturated solution of matrix, α-cyano-4-hydroxy-cinnamic acid (α-HCCA) (Bruker Daltonics). An external calibration was performed by spotting on the 96 calibration positions of the Anchor Chip Target 1 μL of peptide calibration standards (Bruker Daltonics) containing the following peptides: angiotensin II (1046.5420 Da), angiotensin I (1296.6853 Da), substance P (1347.7361 Da), bombesin (1619.8230 Da), ACTH clip 1-17 (2093.0868 Da), ACTH clip 18-39 (2465.1990 Da), somatostatin 28 (3147.4714 Da) and OGP 1.5% (w/v). Samples were analyzed in a MALDI-TOF/TOF spectrometer (Ultraflex, Bruker Daltonics) using an accelerating voltage of 25 kV for the Peptide Mass Fingerprint (PMF) mode. Peptide matching and protein searches were performed automatically with the MASCOT 2.0 software. For the PMF search the parameters were the following: C-carbaimidomethyl (fixed modification), M-oxidation (variable modification), monoisotopic (mass value), 100 ppm (peptide mass tolerance), 1 (max missed cleavage), mammalia (taxonomy). Five matching peptides and at least 10% peptide coverage of the theoretical sequences was the minimal requirement for an identity assignment. The identified proteins were organized with the ProteinScape™ database (Protagen-Bruker Daltonics), checked individually and only mouse proteins or highly homologous sequences from other species were considered.

In conclusion, the EGF receptor plays an important role in various tumor diseases. Its hyperactivity can cause cancer of numerous organs. By detecting early stages of tumor growth a dramatic reduction in the mortality rate of cancer patients can be achieved. To date, though, diagnostic markers for liver cancer, such as alpha-Fetoprotein (AFP) and the Des-Gamma-Carboxyprothrombin (DCP) are insufficient for the definite diagnosis of tumor disease.

The studies according to the invention provide new information on the role of EGF in tumorigenesis. The serum proteomics facilitates the discovery of biomarkers and enables an improved early detection of cancers and therapeutic monitoring in the various treatment strategies.

An EGF-transgenic mouse model has been developed and by using this model the consequences of a changed EGF-signalling in the emergence of liver cancer has been investigated. The mouse model is very similar to the human hepatocellular carcinoma allowing research on the various stages of cancerogenesis.

With the help of genomic platform technologies molecular events referring to the individual stages of tumor development could be investigated. Also, the analyses of tumor specific proteins and serum proteins according to the invention provided beneficial approaches for identifying potential serum biomarkers of liver tumors.

Blood proteins are easily accessible. Therefore, great efforts are undertaken worldwide to seek serum biomarkers for monitoring the course of disease in patients. Changes in the expression of serum-proteins or -peptides are easy to measure even in the early stages of the disease to measure, well in advance before the disease phenotype associated with significant metabolic damage manifests.

A major drawback in serum proteome analytics is, however, the complexity of the sample to be analyzed. For this reason, several additional methods for removing abundant proteins, such as albumin, have been developed to facilitate an identification of the less prevalent and disease associated proteins.

However, the pre-treatment of serum samples is problematic, since proteins that only occur in small quantities are lost. Moreover, the prefractioning of serum proteins is costly in terms of time. Here, the efforts for analyzing the serum proteome while avoiding an pre-fractioning are described.

The EGF2B-transgenic mouse has already been described in a previous publication. For the serum proteome analysis blood serum was obtained both from wild type mice as well as tumor mice.

By using a lysis buffer containing thiourea the serum proteins were extracted and separated by 2D-gelelektrophoresis (2-DE) with the use of two different pH-gradients (3-10, 4-7).

Subsequently to colloidal Coomassie-blue staining the spots were cut from the 2D-gel with a spot cutter. Then, the gel samples were washed, discolored and digested with trypsin.

An over-expression of EGF in transgenic mice was confirmed by RT-PCR. As previously described in detail, HCC-disease was induced by liver specific overexpression of EGF. In the genomic studies, also signal proteins regulated by an excessive EGF-tyrosine kinase activity could be identified.

Using genome wide gene expression analysis, new candidate genes that play a role in tumor formation were identified. Some were changed depending on the degree of differentiation of the tumor, so for example the expression of TGFα and PDGFα. Increased expression of eps-15, a substrate of the EGFR was likewise observed.

In less differentiated tumors, the activation of the RhoC-kinase was particularly pronounced. In all of the tumors the expression of cell cycle-regulating proteins such as junB, c-fos, egr-1, and the survival factor IGFBP1 was significantly increased.

The oncogenomic studies revealed important information about the serum proteome analyses and were affected by the motivation to find new diagnostic biomarkers for HCC.

For overcoming the technical difficulties, the analysis of the serum proteome had to be improved so that as many proteins as possible could be identified after separation with high-resolution 2-DE by mass spectrometric methods (MALDI-MS).

With the help of differential display, tumor-associated serum proteins were discovered in the 2D-gel electrophoresis.

In addition, it was possible to automate the MS-analysis of the trypsin-digested proteins.

The different behavior of matrix peptide mixtures in peptide mass fingerprinting has already been published in numerous studies.

It was further a goal to automate the MS and MS/MS-analyses in order to detect tumor serum proteins in a high-throughput format. For example, with the help of this method the zinc-alpha-2-Glycoprotein could be identified with an outstanding sequence coverage.

This was achieved by the use of α-cyano-4-hydroxycinnamic acid in matrix preparations. In addition, this improved mass spectroscopic method also allows access to information on posttranslational modifications such as phosphorylation and glycosylation.

With the help of this newly developed protocol, a reference list of the serum-proteome was created, which enables an important basis for studies in healthy and tumor diseased subjects.

Table 1. List of the 25 differentially regulated proteins. Proteins are sorted according to their name. NCBI accession number, MASCOT score, percent of sequence coverage and number of identified peptides (Match) are given. Protein function, p-value, mean fold change and frequency of in gel identification are also reported in table 1.
Spots were present in 2 gels, but only 1 spot was cut and analyzed for MALDI mass spectrometry identification.
Isoform

### Supplementary Material 1 (FIG. 5):

Outline of the experiments. Two experiments were carried out. In the experiment 1, n=1 control serum (non transgenic mouse) (C1) was run in duplicate and then studied for the search and identification of proteins; n=3 tumour (T1, T2, T3) sera of bearing mice (EGF) were run in duplicate and then studied for the search and identification of proteins.

In the experiment 2, n=4 control sera (non transgenic mice) (C1, C2, C3, C4) and n=4 tumour sera (T1, T4, T5, T6) were run and then processed for protein identification. In summary a total of 5 gels were studied for the control samples (grey boxes) and a total of 9 gels (grey boxes) were studied for the tumour samples.

(IEF: isoelectric focusing; 2-DE: two dimensional electrophoresis

### Supplementary Material 2 (FIG. 6):

Distribution of relative volumes of identified proteins.

Primary hepatocellular carcinoma (HCC) is worldwide a common neoplasm with approximately 600 000 death per year. Early detection of tumor growth is essential for therapy and overall survival. Disease-associated proteins have been searched in the sera of HCC bearing mice, which specifically developed HCC, as a result of targeted overexpression of epidermal growth factor (EGF). The picture depicts the distribution of the relative volumes of the spots in the quantification of the 2-DE gels

### Supplementary Material 3 (FIG. 7)

The six spots of Apo A-I in 2-D gels.

### Supplementary Material 4 (FIG. 8)

The five spots of Apo H in 2-D gels.

### Supplementary Material 5 (FIG. 9)

Overexpression and ppm values of apolipoprotein E (apo E) in sera of HCC mice. R=ppm ratio (tumour/control).

### Supplementary Material 6 (FIG. 10):

Overexpression and ppm values of apolipoprotein M (apo M) in sera of HCC mice. R=ppm ratio (tumour/control).

### Supplementary Material 7 (FIG. 11):

Overexpression and ppm values of serum amyloid component P (SAP). (A, B): control samples. (D, E): sera of HCC mice, the second SAP isoform, SAP*, is identified in sera of HCC mice only. (C, F): 3-D view of SAP spots in control and HCC mice respectively. R=ppm ratio (tumour/control).

### Supplementary Material 8 (FIG. 12)

Overexpression and ppm values of two A2MG-fragments in sera of HCC mice (E, F, G, H). The x and y isoforms (A, E). R=ppm ratio (tumour/control).

### Supplementary Material 9 (FIG. 13):

Down-regulation of glutathione peroxidase 3 (Gpx3). (A, C): control samples, gpx3 is present in two isoforms, Gpx3* and Gpx3. (B, D): tumour samples, the isoform Gpx3 is down regulated, while the isoform Gpx3* is now virtually absent.

### Supplementary Material 10 (FIG. 14):

(A): Expression of fibrinogens alpha, beta and gamma (Fga, Fgb, Fgg) in sera of HCC mice. (B, C, D): zoom view of the gels; Not all tumor animals carried the three fibrinogens in sera.

### Supplementary Material 11 (FIG. 15):

Down regulation of immunoglobulins. Many immunoglobulins were virtually absent in tumor samples (B).

### Supplementary Table 1. Serum Proteins

A total of 2500 spots derived from 14 gels were excised and digested with trypsin (Promega). Peptides were loaded on a MTP Anchor Chip Target 600/384 (Bruker Daltonics) previously prepared with HCCA and analyzed in a MALDI-TOF-TOF spectrometer (Ultraflex I, Bruker Daltonics). Peptide matching and protein searches were performed automatically with the MASCOT 2.0 software. MASCOT scores are also reported in the table (see column “Mascot Score”). Five matching peptides (see column “N° of matched peptides”) and at least 10%) peptide coverage of the theoretical sequences was the minimal requirement for an identity assignment (see column “Coverage”). The identified proteins were organized with the ProteinScape™ database (Bruker Daltonics) and checked individually.

In the table proteins are sorted by alphabetical order in the second column and the NCBI annotation is given in the first column. The theoretical pl, MW, and biological function are given herein. Expression of proteins and frequency of identification are reported in the column “regulation” and “gels” respectively.

### Supplementary Table 2. Sequences of the Identified Peptides.

TABLE 1

| | | | | N° of | | | Regulation | |
|---|---|---|---|---|---|---|---|---|
| Acc. Number | | Mascot | Sequence | matched | | p- | (tumor/ | |
| (NCBI) | Protein Name | Score | Coverage % | peptides | Function | value | kontrol) | Gels |
| gi\|191765 | Alpha fetoprotein | 234 | 39.2 | 16 | Unknown | 0.035 | 1.6 | 3 |
| gi\|14318646 | Amylase 1, | 72.5 | 23.1 | 13 | Unknown | — | tumor | 3 |
| | salivary | | | | | | | |
| gi\|6753096 | Apolipoprotein | 84.2 | 32.3 | 20 | Reverse transport of cholesterol | — | tumor | 4 |
| | A-I | | | | from tissues to the liver | | | |
| gi\|6753102 | Apolipoprotein | 220 | 60.5 | 18 | Binding and transport | 0.035 | 2.2 | 12 |
| | E | | | | | | | |
| gi\|9055162 | Apolipoprotein | 114 | 45.3 | 9 | Lipid transport. | 0.044 | 8 | 8 |
| | M | | | | | | | |
| gi\|2921308 | Carboxylesterase | 93.3 | 26.4 | 14 | Metabolism of xenobiotics | — | tumor | 2 |
| | precursor | | | | | | | |
| gi\|13899173 | Caspase recruitment | 70.3 | 11.7 | 9 | Promotion of apoptosis. | — | tumor | 2 |
| | domain protein 12 | | | | | | | |
| gi\|49523333 | Clusterin | 79.6 | 22.1 | 10 | Maybe binding to cells, | — | tumor | 4 |
| | (Apolipoprotein | | | | membranes and hydrophobic | | | |
| | J) | | | | proteins. | | | |
| gi\|33563252 | Fibrinogen, alpha | 204 | 47.2 | 26 | Cofactor in platelet aggregation | — | tumor | 1° |
| | polypeptide | | | | | | | |
| gi\|33859809 | Fibrinogen, beta | 263 | 59.9 | 29 | Cofactor in platelet aggregation | — | tumor | 1° |
| | polypeptide | | | | | | | |
| gi\|19527078 | Fibrinogen, gamma | 234 | 60.6 | 22 | Cofactor in platelet aggregation | — | tumor | 1° |
| | polypeptide | | | | | | | |
| gi\|52843238 | Glutathione | 117 | 43 | 9 | Unknown | 0.002 | 0.2 | 4 |
| | peroxidase 3 | | | | | | | |
| gi\|40715898 | HMW-kininogen-II | 106 | 25.5 | 9 | Unknown | — | control | 2 |
| | variant | | | | | | | |
| gi\|21594125 | Lifr protein | 95.5 | 20.4 | 10 | Unknown | — | control | 3 |
| gi\|8569601 | Major urinary | 161 | 73.5 | 11 | Binds pheromones | 0.22 | 0.14 | 3 |
| | protein 1 | | | | | | | |
| gi\|6754654 | Mannose binding | 113 | 45.6 | 10 | Binds mannose and N− | — | control | 5 |
| | lectin (A) | | | | acetylglucosamine in a Ca++ | | | |
| | | | | | dependent way | | | |
| gi\|233018 | Mannose-binding | 160 | 49.6 | 13 | Unknown | — | control | 3 |
| | protein C; MBP-C | | | | | | | |
| gi\|387437 | MHC factor B | 246 | 58.5 | 30 | Complement system | — | tumor | 4 |
| gi\|6679182 | Orosomucoid 1 | 100 | 34.8 | 10 | Acute-phase reaction | — | control | 4 |
| gi\|53787 | Properdin | 143 | 32.5 | 16 | Complement system. | 0.1036 | 0.4 | 6 |
| | (AA 5-441) | | | | | | | |
| gi\|34785996 | A2MG | 216 | 22.3 | 22 | Endopeptidase inhibitor. | 0.0018 | 1.8 | 14 |
| gi\|34785996 | A2MG * | 216 | 22.3 | 22 | Endopeptidase inhibitor. | 2.6 e-5 | 3.2 | 14 |
| gi\|6679383 | Serine (or cysteine) | 124 | 35 | 17 | Inhibitor or plasmin, trypsin, | 0.3033 | 4.7 | 5 |
| | proteinase | | | | and chymotrypsin | | | |
| | inhibitor, clade F, | | | | | | | |
| | member 2; plasmin | | | | | | | |
| | inhibitor alpha 2; | | | | | | | |
| | alpha 2 antiplasmin; | | | | | | | |
| | serine (or cysteine) | | | | | | | |
| | proteinase inhibitor, | | | | | | | |
| | clade F (alpha-2 | | | | | | | |
| | antiplasmin, | | | | | | | |
| | pigment epithelium | | | | | | | |
| | derived factor), | | | | | | | |
| | member 2 [Mus | | | | | | | |
| | musculus] | | | | | | | |
| gi\|38174334 | Serum amyloid | 122 | 37.5 | 9 | Unknown | 0.0103 | 10 | 7 |
| | P-component | | | | | | | |
| gi\|38174334 | Serum amyloid | 92.3 | 27.2 | 7 | Unknown | — | tumor | 4 |
| | P-component * | | | | | | | |

SUPPLEMENTARY TABLE 1

| | | | | | | N° of | | Regulation | |
|---|---|---|---|---|---|---|---|---|---|
| Acc. Number | | | | Mascot | Sequence | matched | | (tumor/ | |
| (NCBI) | Protein Name | pI | Mr (kDa) | Score | Coverage % | peptides | Function | control) | Gels |
| gi\|15277503 | ACTB protein | 5.5 | 40.2 | 249 | 61.4 | 20 | Actins are highly conserved | | 2 |
| | | | | | | | proteins that are involved in various | | |
| | | | | | | | types of cell motility and are | | |
| | | | | | | | ubiquitously expressed in all | | |
| | | | | | | | eukaryotic cells | | |
| gi\|71621 | Actin beta | 5.2 | 41.6 | 192 | 65.2 | 19 | Involved in various types of cell | | 1 |
| | | | | | | | motility. | | |
| gi\|21553101 | Afamin | 5.5 | 69.6 | 146 | 38.1 | 18 | Transport | | 5 |
| gi\|20072386 | Afm protein | 5.6 | 47.6 | 186 | 50.4 | 20 | Transport | | 11 |
| gi\|11277085 | Albumin | 5.4 | 51.4 | 283 | 56.7 | 22 | Serum albumin, the main protein of | | 13 |
| | (fragment) | | | | | | plasma, has a good binding | | |
| | | | | | | | capacity for water, Ca(2+), Na(+), | | |
| | | | | | | | K(+), fatty acids, hormones, | | |
| | | | | | | | bilirubin and drugs. Its main | | |
| | | | | | | | function is the regulation of the | | |
| | | | | | | | colloidal osmotic pressure of blood | | |
| gi\|33859506 | Albumin 1 | 5.7 | 68.7 | 421 | 65.6 | 37 | Transport | | 14 |
| gi\|42542817 | Alpha | 5.6 | 67.3 | 111 | 43 | 18 | Unknown | up | 3 |
| | fetoprotein | | | | | | | regulated | |
| gi\|191765 | Alpha | 5.4 | 47.2 | 234 | 39.2 | 16 | Unknown | up | 9 |
| | fetoprotein | | | | | | | regulated | |
| gi\|191844 | Alpha-1 | 5.3 | 44.8 | 166 | 60.4 | 19 | Inhibitor of serine proteases. Its | | 7 |
| | protease | | | | | | primary target is elastease, but it | | |
| | inhibitor 2 | | | | | | also has a moderate affinity for | | |
| | | | | | | | plasmin and thrombin. | | |
| gi\|31982171 | Alpha-2 | 6 | 165.1 | 131 | 22.6 | 31 | A proteinase activates the inhibitor | | 9 |
| | macroglobulin | | | | | | by specific proteolysis in the bait | | |
| | MUG1 | | | | | | region, which, by an unknown | | |
| | | | | | | | mechanism leads to reaction at the | | |
| | | | | | | | cysteinyl-glutamyl internal thiol | | |
| | | | | | | | ester site and to a conformational | | |
| | | | | | | | change, whereby the proteinase is | | |
| | | | | | | | trapped and/or covalently bound to | | |
| | | | | | | | the inhibitor. While in the tetrameric | | |
| | | | | | | | proteinase inhibitors steric | | |
| | | | | | | | inhibition is sufficiently strong, | | |
| | | | | | | | monomeric forms need a covalent | | |
| | | | | | | | linkage between the activated | | |
| | | | | | | | glutamyl residue of the original thiol | | |
| | | | | | | | ester and a terminal amino group of | | |
| | | | | | | | a lysine or another nucleophilic | | |
| | | | | | | | group on the proteinase, for | | |
| | | | | | | | inhibition to be effective | | |
| gi\|7304875 | Alpha-2-HS- | 6.1 | 37.3 | 98.8 | 48.4 | 13 | Probably involved in differentiation. | | 8 |
| | glycoprotein | | | | | | | | |
| | (Fetuin) | | | | | | | | |
| gi\|14318646 | Amylase 1, | 6.6 | 57.6 | 72.5 | 23.1 | 13 | Unknown | tumor | 3 |
| | salivary | | | | | | | | |
| gi\|425520 | Anti-colorectal | 6.2 | 26.5 | 88.8 | 40.7 | 6 | Unknown | | 1 |
| | carcinoma | | | | | | | | |
| | light chain | | | | | | | | |
| gi\|26345182 | Apolipoprotein | 5.4 | 30.6 | 163 | 62.5 | 18 | Participates in the reverse transport | | 12 |
| | A1 (Unnamed | | | | | | of cholesterol from tissues to the | | |
| | protein | | | | | | liver for excretion by promoting | | |
| | product) | | | | | | cholesterol efflux from tissues and | | |
| | | | | | | | by acting as a cofactor for the | | |
| | | | | | | | lecithin cholesterol acyltransferase | | |
| | | | | | | | (LCAT). | | |
| gi\|14789706 | Apolipoprotein | 5.3 | 45 | 292 | 73.9 | 25 | May have a role in chylomicrons | | 10 |
| | A4 | | | | | | and VLDL secretion and | | |
| | | | | | | | catabolism. Required for efficient | | |
| | | | | | | | activation of lipoprotein lipase by | | |
| | | | | | | | ApoC-II; potent activator of LCAT. | | |
| | | | | | | | Apoa-IV is a major component of | | |
| | | | | | | | HDL and chylomicrons. | | |
| gi\|6753096 | Apolipoprotein | 5.4 | 30.5 | 84.2 | 32.3 | 20 | Participates in the reverse transport | tumor | 4 |
| | A-I | | | | | | of cholesterol from tissues to the | | |
| | | | | | | | liver for excertion by promoting | | |
| | | | | | | | cholesterol efflux from tissues and | | |
| | | | | | | | by acting as a cofactor for the | | |
| | | | | | | | lecithin cholesterol acyltransferase | | |
| | | | | | | | (LCAT). | | |
| gi\|6753102 | Apolipoprotein | 5.7 | 33.2 | 220 | 60.5 | 18 | Mediates the binding, | up | 12 |
| | E | | | | | | internalization, and catabolism of | regulated | |
| | | | | | | | lipoprotein particles. It can serve as | | |
| | | | | | | | a ligand for the LDL (apo B/E) | | |
| | | | | | | | receptor and for the specific apo-E | | |
| | | | | | | | receptor (chylomicron remnant) of | | |
| | | | | | | | hepatic tissues. | | |
| gi\|1938223 | Apolipoprotein | 9.7 | 36.3 | 153 | 59.1 | 15 | Binds to various kinds of negatively | | 12 |
| | H (beta-2 | | | | | | charged substances such as | | |
| | glycoprotein | | | | | | heparin, phospholipids, and | | |
| | I) | | | | | | dextran sulfate. May prevent | | |
| | | | | | | | activation of the intrinsic blood | | |
| | | | | | | | coagulation cascade by binding to | | |
| | | | | | | | phospholipids on the surface of | | |
| | | | | | | | damaged cells. | | |
| gi\|9055162 | Apolipoprotein | 6.1 | 21.3 | 114 | 45.3 | 9 | Probably involved in lipid transport. | up | 8 |
| | M | | | | | | | regulated | |
| gi\|2921308 | Carboxylesterase | 5 | 61.1 | 93.3 | 26.4 | 13 | Involved in the detoxification of | tumor | 2 |
| | precursor | | | | | | xenobiotics and in the activation of | | |
| | | | | | | | ester and amide prodrugs. Involved | | |
| | | | | | | | in the extracellular metabolism of | | |
| | | | | | | | lung surfactant | | |
| gi\|13899173 | Caspase | 6.27 | 116.1 | 70.3 | 11.7 | 9 | Plays a role in the promotion of | tumor | 2 |
| | recruitment | | | | | | apoptosis | | |
| | domain | | | | | | | | |
| | protein 12 | | | | | | | | |
| gi\|49523333 | Clusterin | 5.4 | 51.7 | 79.6 | 22.1 | 10 | Not yet clear. It is known to be | tumor | 4 |
| | (Apolipoprotein | | | | | | expressed on a variety of tissues | | |
| | J) | | | | | | and it seems to be able to bind to | | |
| | | | | | | | cells, membranes and hydrophobic | | |
| | | | | | | | proteins. It has been associated | | |
| | | | | | | | with programmed cell death. | | |
| gi\|6753798 | Coagulation | 6 | 70.3 | 215 | 45 | 24 | Thrombin, which cleaves bonds | | 10 |
| | factor II | | | | | | after Arg and Lys, converts | | |
| | (prothrombin) | | | | | | fibrinogen to fibrin and activates | | |
| | | | | | | | factors V, VII, VIII, XIII, and in | | |
| | | | | | | | complex with thrombomodulin, | | |
| | | | | | | | protein C. | | |
| gi\|13624321 | Coagulation | 6.6 | 76 | 190 | 32.8 | 19 | The B chain of factor XIII is not | | 3 |
| | factor XIII, beta | | | | | | catalytically active, but is thought to | | |
| | subunit | | | | | | stabilize the A subunits and | | |
| | | | | | | | regulate the rate of | | |
| | | | | | | | transglutaminase formation by | | |
| | | | | | | | thrombin. | | |
| gi\|6996919 | Complememnt | 7.1 | 85 | 264 | 52 | 30 | Cell proliferation and complement | | 12 |
| | histocompatibility | | | | | | activation, alternative pathway. | | |
| | 2, complement | | | | | | | | |
| | component | | | | | | | | |
| | factor B | | | | | | | | |
| gi\|28175786 | Complement C3 | 6.4 | 186.4 | 289 | 29 | 40 | C3 plays a central role in the | | 12 |
| | precursor | | | | | | activation of the complement | | |
| | (HSE-MSF) | | | | | | system. Its processing by C3 | | |
| | | | | | | | convertase is the central reaction in | | |
| | | | | | | | both classical and alternative | | |
| | | | | | | | complement pathways. After | | |
| | | | | | | | activation C3b can bind covalently, | | |
| | | | | | | | via its reactive thiolester, to cell | | |
| | | | | | | | surface carbohydrates or immune | | |
| | | | | | | | aggregates. | | |
| gi\|220349 | Complement C4 | 9.1 | 59.2 | 217 | 47.9 | 22 | C4 plays a central role in the | | 8 |
| | | | | | | | activation of the classical pathway | | |
| | | | | | | | of the complement system. It is | | |
| | | | | | | | processed by activated C1 which | | |
| | | | | | | | remove from the alpha chain the | | |
| | | | | | | | C4a anaphylatoxin. | | |
| gi\|309119 | Complement | 6 | 46.6 | 93.9 | 33.8 | 11 | Controls the pathway of | | 1 |
| | C4b-binding | | | | | | complement activation. It | | |
| | protein | | | | | | accelerates the degradation of the | | |
| | precursor | | | | | | C4bC2a complex. It interacts with | | |
| | | | | | | | SAP | | |
| gi\|15030019 | Complement | 5.7 | 86.6 | 207 | 39 | 27 | Involved in the formation of the lytic | | 2 |
| | component 6 | | | | | | c5b-9m complex | | |
| gi\|27462724 | Complement | 4.8 | 76.9 | 72.3 | 18.3 | 9 | C1s B chain is a serine protease | | 1 |
| | component | | | | | | that combines with C1q and C1s to | | |
| | C1SA | | | | | | form C1, the first component of the | | |
| | | | | | | | classical pathway of the | | |
| | | | | | | | complement system. C1r activates | | |
| | | | | | | | C1s so that it can, in turn, activate | | |
| | | | | | | | C2 and C4 | | |
| gi\|19072788 | Complement | 6.6 | 139 | 264 | 32 | 30 | Factor H functions as a cofactor in | | 10 |
| | component | | | | | | the inactivation of C3b by factor I | | |
| | | | | | | | and also increases the rate of | | |
| | factor h | | | | | | dissociation of the C3bBb complex | | |
| | | | | | | | (C3 convertase) and the (C3b)NBB | | |
| | | | | | | | complex (C5 convertase) in the | | |
| | | | | | | | alternative complement pathway | | |
| gi\|6671744 | Complement | 9.2 | 67.2 | 87.6 | 21.7 | 7 | Responsible for cleaving the alpha- | | 6 |
| | component | | | | | | chains of C4b and C3b in the | | |
| | factor i | | | | | | presence of the cofactors C4- | | |
| | | | | | | | binding protein and factor H | | |
| | | | | | | | respectively. | | |
| gi\|9954973 | Complement D | 5 | 31.1 | 75.1 | 38.3 | 10 | C3 plays a central role in the | | 1 |
| | Chain D, N- | | | | | | activation of the complement | | |
| | Terminally | | | | | | system. Its processing by C3 | | |
| | Truncated C3dg | | | | | | convertase is the central reaction in | | |
| | Fragment | | | | | | both classical and alternative | | |
| | | | | | | | complement pathways. After | | |
| | | | | | | | activation C3b can bind covalently, | | |
| | | | | | | | via its reactive thiolester, to cell | | |
| | | | | | | | surface carbohydrates or immune | | |
| | | | | | | | aggregates. Derived from | | |
| | | | | | | | proteolytic degradation of | | |
| | | | | | | | complement C3, C3a | | |
| | | | | | | | anaphylatoxin is a mediator of local | | |
| | | | | | | | inflammatory process. It induces | | |
| | | | | | | | the contraction of smooth muscle, | | |
| | | | | | | | increases vascular permeability | | |
| | | | | | | | and causes histamine release from | | |
| | | | | | | | mast cells and basophilic | | |
| | | | | | | | leukocytes. The short isoform has | | |
| | | | | | | | B-cell stimulatory activity. | | |
| gi\|6754132 | Complement | 5.1 | 37.3 | 217 | 53.2 | 18 | Involved in the presentation of | | 9 |
| | histocompatibility | | | | | | foreign antigens to the immune | | |
| | 2, Q region locus | | | | | | system | | |
| | 10 | | | | | | | | |
| gi\|54173 | Contraspin | 5 | 46.7 | 259 | 60.5 | 22 | Contrapsin inhibits trypsin-like | | 6 |
| | | | | | | | proteases | | |
| gi\|38614350 | Cp protein | 5.5 | 121.1 | 248 | 40.5 | 36 | Ceruloplasmin is a blue, copper- | | 13 |
| | | | | | | | binding (6-7 atoms per molecule) | | |
| | | | | | | | glycoprotein found in plasma. Four | | |
| | | | | | | | possible functions are ferroxidase | | |
| | | | | | | | activity, amine oxidase activity, | | |
| | | | | | | | copper transport and homeostasis, | | |
| | | | | | | | and superoxide dismutase activity. | | |
| gi\|19388017 | Cpn2 protein | 5.5 | 62 | 112 | 29.7 | 11 | The 83 kDa subunit binds and | | 5 |
| | | | | | | | stabilizes the catalytic subunit at 37 | | |
| | | | | | | | degrees Celsius and keeps it in | | |
| | | | | | | | circulation. Under some | | |
| | | | | | | | circumstances it may be an | | |
| | | | | | | | allosteric modifier of the catalytic | | |
| | | | | | | | subunit | | |
| gi\|11055360 | Epidermal growth | 6.8 | 71.4 | 239 | 43.4 | 22 | cell proliferation-cellular | | 9 |
| | factor receptor | | | | | | morphogenesis-epidermal growth | | |
| | isoform 2 | | | | | | factor receptor signaling pathway | | |
| | | | | | | | positive regulation of cell | | |
| | | | | | | | proliferation-protein amino acid | | |
| | | | | | | | autophosphorylation-regulation of | | |
| | | | | | | | peptidyl-tyrosine phosphorylation | | |
| | | | | | | | signal transduction | | |
| gi\|6679689 | Esterase 1 | 4.9 | 61.1 | 164 | 46.8 | 18 | Involved in the detoxification of | | 9 |
| | | | | | | | xenobiotics and in the activation of | | |
| | | | | | | | ester and amide prodrugs. Involved | | |
| | | | | | | | in the extracellular metabolism of | | |
| | | | | | | | lung surfactant | | |
| gi\|33563252 | Fibrinogen, alpha | 7 | 61.3 | 204 | 47.2 | 26 | Fibrinogen has a double function: | tumor | 1° |
| | polypeptide | | | | | | yielding monomers that polymerize | | |
| | | | | | | | into fibrin and acting as a cofactor | | |
| | | | | | | | in platelet aggregation | | |
| gi\|33859809 | Fibrinogen, beta | 6.5 | 54.8 | 263 | 59.9 | 29 | Fibrinogen has a double function: | tumor | 1° |
| | polypeptide | | | | | | yielding monomers that polymerize | | |
| | | | | | | | into fibrin and acting as a cofactor | | |
| | | | | | | | in platelet aggregation | | |
| gi\|19527078 | Fibrinogen, | 5.5 | 49.4 | 234 | 60.6 | 22 | Fibrinogen has a double function: | tumor | 1° |
| | gamma | | | | | | yielding monomers that polymerize | | |
| | polypeptide | | | | | | into fibrin and acting as a cofactor | | |
| | | | | | | | in platelet aggregation | | |
| gi\|1181242 | Fibronectin | 5.2 | 160.9 | 161 | 23.5 | 25 | Fibronectins bind cell surfaces and | | 5 |
| | | | | | | | compounds including collagen, | | |
| | | | | | | | fibrin, heparin, DNA, and actin. | | |
| | | | | | | | They are involved in cell adhesion, | | |
| | | | | | | | cell motility, opsonization, wound | | |
| | | | | | | | healing, and maintenance of cell | | |
| | | | | | | | shape. Interaction with TNR | | |
| | | | | | | | mediates inhibition of cell adhesion | | |
| | | | | | | | and neurite outgrowth. | | |
| gi\|46849812 | Fibronectin 1 | 5.4 | 272.5 | 349 | 31 | 59 | Fibronectins bind cell surfaces and | | 9 |
| | | | | | | | various compounds including | | |
| | | | | | | | collagen. fibrin, heparin, DNA, and | | |
| | | | | | | | actin. Fibronectins are involved in | | |
| | | | | | | | cell adhesion, cell motility, | | |
| | | | | | | | opsonization, wound healing, and | | |
| | | | | | | | maintenance of cell shape. | | |
| | | | | | | | Interaction with TNR mediates | | |
| | | | | | | | inhibition of cell adhesion and | | |
| | | | | | | | neurite outgrowth. | | |
| gi\|28916693 | Gelsolin | 5.8 | 85.9 | 255 | 47.6 | 28 | Calcium-regulated, actin- | | 12 |
| | | | | | | | modulating protein that binds to the | | |
| | | | | | | | plus (or barbed) ends of actin | | |
| | | | | | | | monomers or filaments, preventing | | |
| | | | | | | | monomer exchange (end-blocking | | |
| | | | | | | | or capping). It can promote the | | |
| | | | | | | | assembly of monomers into | | |
| | | | | | | | filaments (nucleation) as well as | | |
| | | | | | | | sever filaments already formed. | | |
| gi\|52843238 | Glutathione | 9.1 | 25.4 | 117 | 43 | 9 | Unknown | down | 4 |
| | peroxidase 3 | | | | | | | regulated | |
| gi\|17512357 | Gpld1 protein | 6.3 | 93.6 | 125 | 19.4 | 16 | This protein hydrolyzes the inositol | | 3 |
| | | | | | | | phosphate linkage in proteins | | |
| | | | | | | | anchored by phosphatidylinositol | | |
| | | | | | | | glycans (GPI-anchor) thus | | |
| | | | | | | | releasing these proteins from the | | |
| | | | | | | | membrane. | | |
| gi\|1694789 | GRS protein | 5.3 | 20.1 | 70 | 44.6 | 5 | Retards apoptosis induced by IL-3 | | 1 |
| | | | | | | | deprivation. May function in the | | |
| | | | | | | | response of hemopoietic cells to | | |
| | | | | | | | external signals and in maintaining | | |
| | | | | | | | endothelial survival during infection | | |
| gi\|37719755 | GUGU alpha | 6.2 | 44.4 | 155 | 50.6 | 16 | Multifunction | | 3 |
| gi\|23956086 | Hemopexin | 9 | 51.3 | 238 | 55.7 | 23 | Binds heme and transports it to the | | 11 |
| | | | | | | | liver for breakdown and iron | | |
| | | | | | | | recovery, after which the free | | |
| | | | | | | | hemopexin returns to the | | |
| | | | | | | | circulation | | |
| gi\|50082914 | High molecular | 4.7 | 53.2 | 89.2 | 27.7 | 11 | (1) Kininogens are inhibitors of thiol | | 3 |
| | weight kininogen | | | | | | proteases; (2) HMW-kininogen | | |
| | I isoform DeltaD5 | | | | | | plays an important role in blood | | |
| | | | | | | | coagulation by helping to position | | |
| | | | | | | | optimally prekallikrein and factor XI | | |
| | | | | | | | next to factor XII; (3) HMW- | | |
| | | | | | | | kininogen inhibits the thrombin- and | | |
| | | | | | | | plasmin-induced aggregation of | | |
| | | | | | | | thrombocytes; (4) the active | | |
| | | | | | | | peptide bradykinin that is released | | |
| | | | | | | | from HMW-kininogen shows a | | |
| | | | | | | | variety of physiological effects: (4A) | | |
| | | | | | | | influence in smooth muscle | | |
| | | | | | | | contraction, (4B) induction of | | |
| | | | | | | | hypotension, (4C) natriuresis and | | |
| | | | | | | | diuresis, (4D) decrease in blood | | |
| | | | | | | | glucose level, (4E) it is a mediator | | |
| | | | | | | | of inflammation and causes (4E1) | | |
| | | | | | | | increase in vascular permeability, | | |
| | | | | | | | (4E2) stimulation of nociceptors | | |
| | | | | | | | (4E3) release of other mediators of | | |
| | | | | | | | inflammation (e.g. prostaglandins), | | |
| | | | | | | | (4F) it has a cardioprotective effect | | |
| | | | | | | | (directly via bradykinin action, | | |
| | | | | | | | indirectly via endothelium-derived | | |
| | | | | | | | relaxing factor action); (5) LMW- | | |
| | | | | | | | kininogen inhibits the aggregation | | |
| | | | | | | | of thrombocytes; (6) LMW- | | |
| | | | | | | | kininogen is in contrast to HMW- | | |
| | | | | | | | kininogen not involved in blood | | |
| | | | | | | | clotting | | |
| gi\|40715898 | HMW-kininogen- | 6 | 51 | 86.4 | 25.8 | 12 | (1) Kininogens are inhibitors of thiol | control | 2 |
| | II variant | | | | | | proteases; (2) HMW-kininogen | | |
| | | | | | | | plays an important role in blood | | |
| | | | | | | | coagulation by helping to position | | |
| | | | | | | | optimally prekallikrein and factor XI | | |
| | | | | | | | next to factor XII; (3) HMW- | | |
| | | | | | | | kininogen inhibits the thrombin- and | | |
| | | | | | | | plasmin-induced aggregation of | | |
| | | | | | | | thrombocytes; (4) the active | | |
| | | | | | | | peptide bradykinin that is released | | |
| | | | | | | | from HMW-kininogen shows a | | |
| | | | | | | | variety of physiological effects: (4A) | | |
| | | | | | | | influence in smooth muscle | | |
| | | | | | | | contraction, (4B) induction of | | |
| | | | | | | | hypotension, (4C) natriuresis and | | |
| | | | | | | | diuresis, (4D) decrease in blood | | |
| | | | | | | | glucose level, (4E) it is a mediator | | |
| | | | | | | | of inflammation and causes (4E1) | | |
| | | | | | | | increase in vascular permeability, | | |
| | | | | | | | (4E2) stimulation of nociceptors | | |
| | | | | | | | (4E3) release of other mediators of | | |
| | | | | | | | inflammation (e.g. prostaglandins), | | |
| | | | | | | | (4F) it has a cardioprotective effect | | |
| | | | | | | | (directly via bradykinin action, | | |
| | | | | | | | indirectly via endothelium-derived | | |
| | | | | | | | relaxing factor action); (5) LMW- | | |
| | | | | | | | kininogen inhibits the aggregation | | |
| | | | | | | | of thrombocytes; (6) LMW- | | |
| | | | | | | | kininogen is in contrast to HMW- | | |
| | | | | | | | kininogen not involved in blood | | |
| | | | | | | | clotting | | |
| gi\|31615671 | Ig (A Chain A, | 5.5 | 23.6 | 76.4 | 44.4 | 6 | Host defense | control | 3 |
| | Crystal Structure | | | | | | | | |
| | Of Fab Fragment | | | | | | | | |
| | Of Antibody | | | | | | | | |
| | Hyhel-26 | | | | | | | | |
| | Complexed with | | | | | | | | |
| | Lysozyme) | | | | | | | | |
| gi\|4930001 | Ig (A Chain A, | 6 | 23.7 | 65.8 | 41.6 | 7 | Host defense | control | 4 |
| | Idiotope-Anti- | | | | | | | | |
| | Idiotope Fab- | | | | | | | | |
| | Fab Complex) | | | | | | | | |
| gi\|11514687 | Ig (A Chain A, | 7.4 | 23.6 | 78.7 | 49.3 | 6 | Host defense | control | 1 |
| | Lyme Disease | | | | | | | | |
| | Antigen Ospa In | | | | | | | | |
| | Complex With | | | | | | | | |
| | Neutralizing | | | | | | | | |
| | Antibody Fab | | | | | | | | |
| | La-2) | | | | | | | | |
| gi\|42543442 | Ig (A Chain A, | 9.4 | 24.2 | 89.2 | 44.7 | 6 | Host defense | control | 2 |
| | S25-2-Kdo | | | | | | | | |
| | Monosaccharide | | | | | | | | |
| | Complex) | | | | | | | | |
| gi\|7766934 | Ig (A Chain A, | 6 | 23.8 | 78.2 | 42.9 | 6 | Host defense | control | 1 |
| | Structure Of An | | | | | | | | |
| | Activity | | | | | | | | |
| | Supressing Fab | | | | | | | | |
| | Fragment To | | | | | | | | |
| | Cytochrome | | | | | | | | |
| | P450 Aromatase) | | | | | | | | |
| gi\|27373551 | Ig (antibody | 7 | 12.7 | 68.5 | 48.7 | 5 | Host defense | tumor | 1 |
| | variable domain) | | | | | | | | |
| gi\|1870378 | Ig (Anti-DNA | 6.3 | 11.6 | 109 | 57 | 6 | Host defense | up | 5 |
| | immunoglobulin | | | | | | | regulated | |
| | light chain IgG) | | | | | | | | |
| gi\|12002896 | Ig (anti-human | 7.4 | 23.5 | 82.4 | 44.6 | 7 | Host defense | control | 1 |
| | apolipoprotein | | | | | | | | |
| | A monoclonal | | | | | | | | |
| | antibody | | | | | | | | |
| | mAb(a)23L kappa | | | | | | | | |
| | light chain) | | | | | | | | |
| gi\|349893 | Ig (C Chain C, | 7.8 | 24 | 114 | 47.5 | 8 | Host defense | control | 1 |
| | Fab (Igg2a, | | | | | | | | |
| | Kappa) Fragment | | | | | | | | |
| | (26-10) Complex | | | | | | | | |
| | With Digoxin) | | | | | | | | |
| gi\|47059057 | Ig (gamma-2b- | 8.9 | 51.9 | 59 | 29 | 8 | Host defense | tumor | 2 |
| | immunoglobulin) | | | | | | | | |
| gi\|1806128 | Ig | 9.3 | 51.7 | 88.4 | 41.9 | 12 | Host defense | | 5 |
| | (immunoglobulin | | | | | | | | |
| | constant heavy | | | | | | | | |
| | chain) | | | | | | | | |
| gi\|10121892 | Ig | 5.2 | 26 | 76.9 | 42.1 | 7 | Host defense | control | 3 |
| | (immunoglobulin | | | | | | | | |
| | IgM MP-18-3-117 | | | | | | | | |
| | kappa light chain) | | | | | | | | |
| gi\|196723 | Ig | 5.6 | 10.9 | 125 | 61.6 | 7 | Host defense | control | 2 |
| | (immunoglobulin | | | | | | | | |
| | kappa- | | | | | | | | |
| | chain VK-1) | | | | | | | | |
| gi\|18033701 | Ig | 5.5 | 11.3 | 65.6 | 70.2 | 5 | Host defense | control | 1 |
| | (immunoglobulin | | | | | | | | |
| | light chain | | | | | | | | |
| | constant region) | | | | | | | | |
| gi\|13399686 | Ig (L Chain L, | 6 | 23.9 | 115 | 70.5 | 10 | Host defense | control | 5 |
| | 64m-2 Antibody | | | | | | | | |
| | Fab Complexed | | | | | | | | |
| | With D | | | | | | | | |
| | (5ht)(6-4)t) | | | | | | | | |
| gi\|3212470 | Ig (L Chain L, | 5.7 | 23.1 | 104 | 58.6 | 5 | Host defense | control | 2 |
| | Anti Taq | | | | | | | | |
| | Fab Tp7) | | | | | | | | |
| gi\|5542523 | Ig (L Chain L, | 9.3 | 24.1 | 107 | 66.2 | 11 | Host defense | control | 2 |
| | Bactericidal | | | | | | | | |
| | Antibody Against | | | | | | | | |
| | Neisseria | | | | | | | | |
| | Meningitidis) | | | | | | | | |
| gi\|16975338 | Ig (L Chain L, | 6.6 | 23.6 | 102 | 69.2 | 11 | Host defense | control | 2 |
| | Crystal Structure | | | | | | | | |
| | Of | | | | | | | | |
| | Immunoglobulin | | | | | | | | |
| | Fab Fragment | | | | | | | | |
| | Complexed | | | | | | | | |
| | With 17-Beta- | | | | | | | | |
| | Estradiol) | | | | | | | | |
| gi\|18655521 | Ig (L Chain L, | 4.8 | 23.9 | 81.1 | 56.4 | 8 | Host defense | | 2 |
| | Crystal Structure | | | | | | | | |
| | Of The Fab | | | | | | | | |
| | Fragment Of The | | | | | | | | |
| | Mouse Anti- | | | | | | | | |
| | Human Fas | | | | | | | | |
| | Antibody Hfe7a) | | | | | | | | |
| gi\|1942810 | Ig (L Chain L, | 6.4 | 24.2 | 124 | 80 | 12 | Host defense | control | 2 |
| | Fab Fragment | | | | | | | | |
| | Of A Neutralizing | | | | | | | | |
| | Antibody | | | | | | | | |
| | Directed Against | | | | | | | | |
| | An Epitope | | | | | | | | |
| | Of Gp41 From | | | | | | | | |
| | Hiv-1) | | | | | | | | |
| gi\|7546516 | Ig (L Chain L, | 5.6 | 23.7 | 72.4 | 37 | 5 | Host defense | control | 1 |
| | Fab Fragment Of | | | | | | | | |
| | Neutralising | | | | | | | | |
| | Monoclonal | | | | | | | | |
| | Antibody 4c4 | | | | | | | | |
| | Complexed With | | | | | | | | |
| | G-H Loop From | | | | | | | | |
| | Fmdv) | | | | | | | | |
| gi\|17943084 | Ig (L Chain L, | 7.8 | 24.2 | 118 | 59.1 | 8 | Host defense | control | 2 |
| | Structural Basis | | | | | | | | |
| | For Disfavored | | | | | | | | |
| | Elimination | | | | | | | | |
| | Reaction In | | | | | | | | |
| | Catalytic | | | | | | | | |
| | Antibody 1d4) | | | | | | | | |
| gi\|2624723 | Ig (L Chain L, | 7.3 | 24 | 69.9 | 45.7 | 8 | Host defense | control | 1 |
| | Structure Of A | | | | | | | | |
| | Catalytic | | | | | | | | |
| | Antibody, Igg2a | | | | | | | | |
| | Fab Fragment) | | | | | | | | |
| gi\|32263981 | Ig (mAb | 9.9 | 11.6 | 80.9 | 59.9 | 7 | Host defense | control | 1 |
| | immunoglobulin | | | | | | | | |
| | light chain | | | | | | | | |
| | variable region) | | | | | | | | |
| gi\|18568342 | Ig (monoclonal | 6.5 | 26.1 | 77.8 | 51.7 | 8 | Host defense | control | 1 |
| | antibody BBK-2 | | | | | | | | |
| | light chain) | | | | | | | | |
| gi\|52382 | Ig (mu(b) | 6.4 | 50 | 153 | 40.7 | 19 | Host defense | control | 1 |
| | immunoglobulin | | | | | | | | |
| | heavy chain) | | | | | | | | |
| gi\|7439167 | Ig (PC4436 | 8.7 | 49.3 | 92.6 | 29.1 | 8 | Host defense | | 2 |
| | monoclonal | | | | | | | | |
| | antibody 13-1 | | | | | | | | |
| | heavy chain) | | | | | | | | |
| gi\|15824610 | Ig (Pterin- | 7.8 | 12 | 75.4 | 77.5 | 5 | Unknown | control | 1 |
| | mimicking anti- | | | | | | | | |
| | idiotope kappa | | | | | | | | |
| | chain variable | | | | | | | | |
| | region) | | | | | | | | |
| gi\|780265 | Ig gamma-1 chain | 9 | 17.3 | 70.5 | 38.8 | 8 | Host defense | | 3 |
| | C region (15C5) | | | | | | | | |
| | (fragment) | | | | | | | | |
| gi\|1799551 | Ig gamma-chain, | 9.4 | 36.2 | 88.5 | 31 | 9 | Host defense | | 1 |
| | secrete-type | | | | | | | | |
| gi\|223428 | Ig H-C allotype | 7.8 | 36.6 | 174 | 56 | 19 | Host defense | | 9 |
| | gamma2b | | | | | | | | |
| gi\|224008 | Ig J chain | 4.4 | 15.8 | 111 | 42.4 | 9 | Host defense | control | 2 |
| gi\|12832551 | Ig K (unnamed | 6.4 | 23.2 | 98 | 43.1 | 8 | Host defense | control | 2 |
| | protein product) | | | | | | | | |
| gi\|896077 | Ig kappa chain | 7.6 | 8.7 | 90.1 | 74.7 | 6 | Host defense | control | 3 |
| gi\|896089 | Ig kappa chain | 9.5 | 10.5 | 82 | 82.3 | 6 | Host defense | control | 1 |
| gi\|896103 | Ig kappa chain | 4.3 | 10.3 | 64.7 | 57.9 | 5 | Host defense | control | 3 |
| gi\|896107 | Ig kappa chain | 4.6 | 9.1 | 71.6 | 71.8 | 5 | Host defense | control | 1 |
| gi\|7513693 | Ig kappa chain | 6.4 | 23.6 | 104 | 58.9 | 11 | Host defense | control | 1 |
| | (Mab03-1) | | | | | | | | |
| gi\|110429 | Ig kappa chain V | 9.1 | 10.4 | 73.4 | 58.5 | 6 | Host defense | control | 1 |
| | region (9.42 | | | | | | | | |
| gi\|49258884 | Ig kappa chain V | 9.3 | 11.8 | 70.1 | 50.9 | 7 | Host defense | control | 3 |
| | region (D444) | | | | | | | | |
| gi\|930226 | Ig kappa chain V | 5.1 | 11 | 90 | 55.6 | 8 | Host defense | control | 2 |
| | region (hybridoma | | | | | | | | |
| | NC19-E11) | | | | | | | | |
| gi\|125796 | Ig kappa chain V- | 5.2 | 12 | 73.7 | 66.7 | 6 | Host defense | down | 1 |
| | III region PC | | | | | | | regulated | |
| | 2880/PC 1229 | | | | | | | | |
| gi\|197064 | Ig kappa chain V- | 7.3 | 11.9 | 92.3 | 99.1 | 7 | Host defense | control | 1 |
| | region (V-Jk1) | | | | | | | | |
| | protein | | | | | | | | |
| gi\|1613779 | Ig kappa light | 5.5 | 23.9 | 69.6 | 38.3 | 10 | Host defense | control | 1 |
| | chain | | | | | | | | |
| gi\|284924 | Ig light chain V | 6.1 | 10.7 | 86.9 | 75.5 | 6 | Host defense | tumor | 1 |
| | region (clone | | | | | | | | |
| | 185-c1) | | | | | | | | |
| gi\|494164 | Igg2a Fab | 5.7 | 23.9 | 65.7 | 39.6 | 5 | Host defense | control | 1 |
| | Fragment (Fab | | | | | | | | |
| | 179) | | | | | | | | |
| gi\|229901 | Igg2b, Kappa | 5.8 | 23.7 | 108 | 70.1 | 12 | Host defense | control | 5 |
| gi\|62204734 | Inter alpha- | 6.1 | 104 | 220 | 37 | 29 | Unknown | | 5 |
| | trypsin inhibitor, | | | | | | | | |
| | heavy chain 4 | | | | | | | | |
| gi\|16418335 | Leucine-rich | 6 | 37.4 | 71 | 23.4 | 8 | Unknown | | 3 |
| | alpha-2- | | | | | | | | |
| | glycoprotein | | | | | | | | |
| gi\|21594125 | Lifr protein | 6.5 | 81.3 | 95.5 | 20.4 | 10 | The WSXWS motif appears to be | control | 3 |
| | (leukemia | | | | | | necessary for proper protein folding | | |
| | inhibitory factor | | | | | | and thereby efficient intracellular | | |
| | receptor) | | | | | | transport and cell-surface receptor | | |
| | | | | | | | binding | | |
| gi\|8569601 | Major urinary | 4.9 | 20.6 | 161 | 73.5 | 11 | Binds pheromones | down | 3 |
| | protein 1 | | | | | | | regulated | |
| gi\|6754654 | Mannose binding | 7.3 | 25.4 | 113 | 45.6 | 10 | Binds mannose and N− | control | 5 |
| | lectin (A) | | | | | | acetylglucosamine in a Ca++ | | |
| | | | | | | | dependent way. Is capable of host | | |
| | | | | | | | defense against pathogens, by | | |
| | | | | | | | activating the classical complement | | |
| | | | | | | | pathway independently of the | | |
| | | | | | | | antibody | | |
| gi\|233018 | Mannose binding | 4.9 | 25.9 | 160 | 49.6 | 11 | Binds mannose and N− | control | 3 |
| | protein C | | | | | | acetylglucosamine. Host defense | | |
| | | | | | | | against pathogens, by activating | | |
| | | | | | | | the complement pathway | | |
| | | | | | | | independently of the antibody. | | |
| gi\|2118830 | MHC class I | 5.1 | 37 | 240 | 53.7 | 21 | Involved in the presentation of | | 9 |
| | histocompatibility | | | | | | foreign antigens to the immune | | |
| | antigen H-2 Q10 | | | | | | system | | |
| | alpha chain | | | | | | | | |
| gi\|387437 | MHC factor B | 8.7 | 54 | 246 | 58.5 | 30 | Factor B which is part of the | tumor | 4 |
| | | | | | | | alternate pathway of the | | |
| | | | | | | | complement system is cleaved by | | |
| | | | | | | | factor D into 2 fragments: Ba and | | |
| | | | | | | | Bb. Bb, a serine protease, then | | |
| | | | | | | | combines with complement factor | | |
| | | | | | | | 3b to generate the C3 or C5 | | |
| | | | | | | | convertase | | |
| gi\|55217 | Murine valosin- | 5.1 | 89.3 | 91.6 | 19.1 | 12 | Necessary for the fragmentation of | | 1 |
| | containing | | | | | | Golgi stacks during mitosis and for | | |
| | protein | | | | | | their reassembly after mitosis. | | |
| | | | | | | | Involved in the formation of the | | |
| | | | | | | | transitional endoplasmic reticulum | | |
| | | | | | | | (tER). The transfer of membranes | | |
| | | | | | | | from the endoplasmic reticulum to | | |
| | | | | | | | the Golgi apparatus occurs via 50- | | |
| | | | | | | | 70 nm transition vesicles which | | |
| | | | | | | | derive from part-rough, part-smooth | | |
| | | | | | | | transitional elements of the | | |
| | | | | | | | endoplasmic reticulum (tER). | | |
| | | | | | | | Vesicle budding from the tER is an | | |
| | | | | | | | ATP-dependent process. The | | |
| | | | | | | | ternary complex containing UFD1L, | | |
| | | | | | | | VCP and NPL4 binds ubiquitinated | | |
| | | | | | | | proteins and is necessary for the | | |
| | | | | | | | export of misfolded proteins from | | |
| | | | | | | | the ER to the cytoplasm, where | | |
| | | | | | | | they are degraded by the | | |
| | | | | | | | proteasome. The NPL4-UFD1L- | | |
| | | | | | | | VCP complex regulates spindle | | |
| | | | | | | | disassembly at the end of mitosis | | |
| | | | | | | | and is necessary for the formation | | |
| | | | | | | | of a closed nuclear envelope | | |
| gi\|6679182 | Orosomucoid 1 | 5.4 | 23.9 | 100 | 34.8 | 10 | Appears to function in modulating | control | 4 |
| | | | | | | | the activity of the immune system | | |
| | | | | | | | during the acute-phase reaction | | |
| gi\|31982113 | Plasminogen | 6.1 | 90.8 | 449 | 61.5 | 53 | Plasmin dissolves the fibrin of | | 10 |
| | [Mus musculus] | | | | | | blood clots and acts as a | | |
| | | | | | | | proteolytic factor in a variety of | | |
| | | | | | | | other processes including | | |
| | | | | | | | embryonic development, tissue | | |
| | | | | | | | remodeling, tumor invasion, and | | |
| | | | | | | | inflammation; in ovulation it | | |
| | | | | | | | weakens the walls of the Graafian | | |
| | | | | | | | follicle. It activates the urokinase- | | |
| | | | | | | | type plasminogen activator, | | |
| | | | | | | | collagenases and several | | |
| | | | | | | | complement zymogens, such as | | |
| | | | | | | | C1 and C5. It cleaves fibrin, | | |
| | | | | | | | fibronectin, thrombospondin, | | |
| | | | | | | | laminin and von Willebrand factor. | | |
| | | | | | | | Its role in tissue remodeling and | | |
| | | | | | | | tumor invasion may be modulated | | |
| | | | | | | | by CSPG4 | | |
| gi\|2144495 | PLMS plasmin | 6 | 90.8 | 352 | 51.4 | 38 | Plasmin dissolves the fibrin of | n.v.o. | 8 |
| | | | | | | | blood clots and acts as a | | |
| | | | | | | | proteolytic factor in a variety of | | |
| | | | | | | | other processes including | | |
| | | | | | | | embryonic development, tissue | | |
| | | | | | | | remodeling, tumor invasion, and | | |
| | | | | | | | inflammation; in ovulation it | | |
| | | | | | | | weakens the walls of the Graafian | | |
| | | | | | | | follicle. It activates the urokinase- | | |
| | | | | | | | type plasminogen activator, | | |
| | | | | | | | collagenases and several | | |
| | | | | | | | complement zymogens, such as | | |
| | | | | | | | C1 and C5. It cleaves fibrin, | | |
| | | | | | | | fibronectin, thrombospondin, | | |
| | | | | | | | laminin and von Willebrand factor. | | |
| | | | | | | | Its role in tissue remodeling and | | |
| | | | | | | | tumor invasion may be modulated | | |
| | | | | | | | by CSPG4 | | |
| gi\|6680608 | Pregnancy zone | 6.2 | 165.8 | 113 | 13.7 | 14 | Endopeptidase inhibitor activity | | 8 |
| | protein | | | | | | | | |
| gi\|53787 | Properdin | 8.4 | 47.5 | 143 | 32.5 | 16 | A positive regulator of the alternate | up | 6 |
| | (AA 5-441) | | | | | | pathway of complement. It binds to | regulated | |
| | | | | | | | and stabilizes the C3-and C5- | | |
| | | | | | | | convertase enzyme complexes. | | |
| gi\|49868 | Put. beta-actin | 5.7 | 39.2 | 136 | 44.4 | 16 | Involved in various types of cell | | 1 |
| | (aa 27-375) | | | | | | motility. | | |
| gi\|34785996 | Pzp protein | 6.2 | 165.9 | 187 | 28.2 | 32 | Unknown | up | 14 |
| | | | | | | | | regulated | |
| gi\|33859612 | Retinol binding | 5.6 | 23.2 | 147 | 51.7 | 14 | Delivers retinol from the liver stores | | 9 |
| | protein 4 | | | | | | to the peripheral tissues. In plasma, | | |
| | | | | | | | the RBP-retinol complex interacts | | |
| | | | | | | | with transthyretin, this prevents its | | |
| | | | | | | | loss by filtration through the kidney | | |
| | | | | | | | glomeruli. | | |
| gi\|6678083 | Serine (or | 5.3 | 45.9 | 149 | 62.7 | 20 | Inhibitor of serine proteases. Can | | 6 |
| | cysteine) | | | | | | inhibit trypsin and chymotrypsin; | | |
| | proteinase | | | | | | relatively ineffective against | | |
| | inhibitor, clade A, | | | | | | elastase | | |
| | member 1a | | | | | | | | |
| gi\|15029662 | Serine (or | 5.3 | 46 | 143 | 51.6 | 17 | Inhibitor of serine proteases. Its | | 8 |
| | cysteine) | | | | | | primary target is elastase, but it | | |
| | proteinase | | | | | | also has a moderate affinity for | | |
| | inhibitor, clade A, | | | | | | plasmin and thrombin. | | |
| | member 1a | | | | | | | | |
| gi\|6678085 | Serine (or | 5.2 | 46 | 171 | 47 | 14 | Inhibitor of serine proteases. Can | | 1 |
| | cysteine) | | | | | | inhibit trypsin and chymotrypsin; | | |
| | proteinase | | | | | | relatively ineffective against | | |
| | inhibitor, clade A, | | | | | | elastase | | |
| | member 1d | | | | | | | | |
| gi\|6678087 | Serine (or | 5.4 | 45.9 | 154 | 41.6 | 19 | Does not inhibit elastase or | | 5 |
| | cysteine) | | | | | | chymotrypsin. No target protease | | |
| | proteinase | | | | | | has been identified to date | | |
| | inhibitor, clade A, | | | | | | | | |
| | member 1e | | | | | | | | |
| gi\|18044689 | Serine (or | 5 | 46.9 | 99.1 | 40 | 13 | serine-type endopeptidase inhibitor | | 1 |
| | cysteine) | | | | | | activity | | |
| | proteinase | | | | | | | | |
| | inhibitor, clade A, | | | | | | | | |
| | member 3K | | | | | | | | |
| gi\|18252782 | Serine (or | 6.1 | 52 | 195 | 61.7 | 25 | Most important serine protease | | 7 |
| | cysteine) | | | | | | inhibitor in plasma that regulates | | |
| | proteinase | | | | | | the blood coagulation cascade. AT- | | |
| | inhibitor, clade C | | | | | | III inhibits thrombin as well as | | |
| | (antithrombin), | | | | | | factors IXa, Xa, and XIa. Its | | |
| | member 1 | | | | | | inhibitory activity is greatly | | |
| | | | | | | | enhanced in the presence of | | |
| | | | | | | | heparin. | | |
| gi\|6679383 | Serine (or | 5.8 | 54.9 | 106 | 34.6 | 17 | Serine-type endopeptidase inhibitor | up | 5 |
| | cysteine) | | | | | | activity | regulated | |
| | proteinase | | | | | | | | |
| | inhibitor, clade F, | | | | | | | | |
| | member 2 | | | | | | | | |
| gi\|15929675 | Serpina1a protein | 5.4 | 45.6 | 219 | 62 | 23 | Inhibitor of serine proteases. Can | | 12 |
| | | | | | | | inhibit trypsin and chymotrypsin; | | |
| | | | | | | | relatively ineffective against | | |
| | | | | | | | elastase. | | |
| gi\|14602605 | Serpina1a protein | 5.3 | 46 | 224 | 62 | 24 | Inhibitor of serine proteases. Can | | 7 |
| | | | | | | | inhibit trypsin and chymotrypsin; | | |
| | | | | | | | relatively ineffective against | | |
| | | | | | | | elastase. | | |
| gi\|38174334 | Serum amyloid | 6.2 | 26.2 | 122 | 37.5 | 9 | Unknown | up | 7 |
| | P-component | | | | | | | regulated | |
| gi\|20330802 | Transferrin | 6.8 | 76.7 | 159 | 40 | 47 | Transferrins are iron binding | | 13 |
| | | | | | | | transport proteins which can bind | | |
| | | | | | | | two Fe(3+) ions in associations with | | |
| | | | | | | | the binding of an anion, usually | | |
| | | | | | | | bicarbonate. It is responsible for | | |
| | | | | | | | the transport of iron from sites of | | |
| | | | | | | | absorption and heme degradation | | |
| | | | | | | | to those of storage and utilization. | | |
| | | | | | | | Serum transferrin may also have a | | |
| | | | | | | | further role in stimulating cell | | |
| | | | | | | | proliferation. | | |
| gi\|19354093 | Transthyretin | 5.5 | 15.8 | 155 | 96.6 | 11 | Thyroid hormone-binding protein. | | 8 |
| | | | | | | | Probably transports thyroxine from | | |
| | | | | | | | the bloodstream to the brain. | | |
| gi\|111243 | Vitamin D- | 5.2 | 53.1 | 288 | 65.3 | 30 | Multifunction | | 12 |
| | binding protein | | | | | | | | |
| gi\|1083568 | Zinc-alpha 2- | 5.7 | 33.5 | 94.6 | 40 | 10 | Stimulates lipid degradation in | | 1 |
| | glycoprotein | | | | | | adipocytes and causes the | | |
| | | | | | | | extensive fat losses associated | | |
| | | | | | | | with some advanced cancers | | |

SUPPLEMENTARY TABLE 2

| NCBI Number | Protein | Identified Peptides |
|---|---|---|
| gi\|15277503 | ACTB protein | AGFAGDDAPR |
| | | (SEQ ID NO 3) |
| | | AVFPSIVGRPR |
| | | (SEQ ID NO 4) |
| | | DLYANTVLSGGTTMYPGIADR |
| | | (SEQ ID NO 5) |
| | | DSYVGDEAQSKR |
| | | (SEQ ID NO 6) |
| | | GYSFTTTAER |
| | | (SEQ ID NO 7) |
| | | GYSFTTTAEREIVR |
| | | (SEQ ID NO 8) |
| | | HQGVMVGMGQK ox ox |
| | | (SEQ ID NO 9) |
| | | HQGVMVGMGQKDSYVGDEAQSK ox ox |
| | | (SEQ ID NO 10) |
| | | IIAPPERK |
| | | (SEQ ID NO 11) |
| | | IKIIAPPER |
| | | (SEQ ID NO 12) |
| | | ILTERGYSFTTTAER |
| | | (SEQ ID NO 13) |
| | | IWHHTFYNELR |
| | | (SEQ ID NO 14) |
| | | KDLYANTVLSGGTTMYPGIADR ox |
| | | (SEQ ID NO 15) |
| | | LCYVALDFEQEMATAASSSSLEK cam ox |
| | | (SEQ ID NO 16) |
| | | LDLAGRDLTDYLMK |
| | | (SEQ ID NO 17) |
| | | LDLAGRDLTDYLMK ox |
| | | (SEQ ID NO 18) |
| | | QEYDESGPSIVHR |
| | | (SEQ ID NO 19) |
| | | QEYDESGPSIVHRK |
| | | (SEQ ID NO 20) |
| | | SYELPDGQVITIGNER |
| | | (SEQ ID NO 21) |
| | | TTGIVMDSGDGVTHTVPIYEGYALPHAILR ox |
| | | (SEQ ID NO 22) |
| | | VAPEEHPVLLTEAPLNPK |
| | | (SEQ ID NO 23) |
| gi\|71621 | Actin beta | AGFAGDDAPR |
| | | (SEQ ID NO 24) |
| | | AVFPSIVGRPR |
| | | (SEQ ID NO 25) |
| | | DDDIAALVVDNGSGMCK ox cam |
| | | (SEQ ID NO 26) |
| | | DLYANTVLSGGTTMYPGIADR ox |
| | | (SEQ ID NO 27) |
| | | DSYVGDEAQSKR |
| | | (SEQ ID NO 28) |
| | | EITALAPSTMK ox |
| | | (SEQ ID NO 29) |
| | | GYSFTTTAER |
| | | (SEQ ID NO 30) |
| | | GYSFTTTAEREIVR |
| | | (SEQ ID NO 31) |
| | | HQGVMVGMGQK ox |
| | | (SEQ ID NO 32) |
| | | IIAPPERK |
| | | (SEQ ID NO 33) |
| | | IKIIAPPER |
| | | (SEQ ID NO 34) |
| | | IWHHTFYNELR |
| | | (SEQ ID NO 35) |
| | | KDLYANTVLSGGTTMYPGIADR ox |
| | | (SEQ ID NO 36) |
| | | LCYVALDFEQEMATAASSSSLEK cam ox |
| | | (SEQ ID NO 37) |
| | | LDLAGRDLTDYLMK ox |
| | | (SEQ ID NO 38) |
| | | QEYDESGPSIVHR |
| | | (SEQ ID NO 39) |
| | | QEYDESGPSIVHRK |
| | | (SEQ ID NO 40) |
| | | SYELPDGQVITIGNER |
| | | (SEQ ID NO 41) |
| | | TTGIVMDSGDGVTHTVPIYEGYALPHAILR ox |
| | | (SEQ ID NO 42) |
| | | VAPEEHPVLLTEAPLNPK |
| | | (SEQ ID NO 43) |
| gi\|21553101 | Afamin | AAPQLPMEELVSLSK |
| | | (SEQ ID NO 44) |
| | | ANVGFLPPFPTLDPEEK |
| | | (SEQ ID NO 45) |
| | | SLAMVQQECNQFQELGKDTLQR ox cam |
| | | (SEQ ID NO 46) |
| | | CWADNTLPECSK cam cam |
| | | (SEQ ID NO 47) |
| | | DRCWADNTLPECSK cam cam |
| | | (SEQ ID NO 48) |
| | | DSDPDKFFAEFIYEYSR |
| | | (SEQ ID NO 49) |
| | | EACIINANKDDRPEGLSLR cam |
| | | (SEQ ID NO 50) |
| | | FTAAREECSEVQEPESCFSPESSK cam cam |
| | | (SEQ ID NO 51) |
| | | FTESENVCQER cam |
| | | (SEQ ID NO 52) |
| | | HFLVKFTK |
| | | (SEQ ID NO 53) |
| | | IGFKDLTTLLEDVSSMYEGCCEGDVVHCIR cam cam |
| | | cam |
| | | (SEQ ID NO 54) |
| | | KANVGFLPPFPTLDPEEK |
| | | (SEQ ID NO 55) |
| | | RLCFFYNK cam |
| | | (SEQ ID NO 56) |
| | | TINPAVDHCCK cam cam |
| | | (SEQ ID NO 57) |
| | | TINPAVDHCCKTDFAFR cam cam |
| | | (SEQ ID NO 58) |
| | | VLNSINVAVFSK |
| | | (SEQ ID NO 59) |
| | | VMLDYRDR |
| | | (SEQ ID NO 60) |
| | | VMLDYRDR ox |
| | | (SEQ ID NO 61) |
| | | VYMDFLEDCCSR ox cam cam |
| | | (SEQ ID NO 62) |
| gi\|20072386 | Afm protein | ANVGFLPPFPTLDPEEK |
| | | (SEQ ID NO 63) |
| | | ASSSYQRNVCGALIK cam |
| | | (SEQ ID NO 64) |
| | | ATCFQAKAAPITQYLK cam |
| | | (SEQ ID NO 65) |
| | | CWADNTLPECSK cam cam |
| | | (SEQ ID NO 66) |
| | | DDRPEGLSLREAK |
| | | (SEQ ID NO 67) |
| | | DSDPDKFFAEFIYEYSR |
| | | (SEQ ID NO 68) |
| | | EACIINANKDDRPEGLSLR cam |
| | | (SEQ ID NO 69) |
| | | EAKFTESENVCQER cam |
| | | (SEQ ID NO 70) |
| | | FTESENVCQER cam |
| | | (SEQ ID NO 71) |
| | | ITKVYMDFLEDCCSR ox cam cam |
| | | (SEQ ID NO 72) |
| | | KANVGFLPPFPTLDPEEK |
| | | (SEQ ID NO 73) |
| | | LCFFYNKK cam |
| | | (SEQ ID NO 74) |
| | | NVCGALIKFGPK cam |
| | | (SEQ ID NO 75) |
| | | QFQELGKDTLQR |
| | | (SEQ ID NO 76) |
| | | RHPDLSTPELLR |
| | | (SEQ ID NO 77) |
| | | SLAMVQQECK ox cam |
| | | (SEQ ID NO 78) |
| | | SQSQVVNHICSK cam |
| | | (SEQ ID NO 79) |
| | | SQSQVVNHICSKQDSISSK cam |
| | | (SEQ ID NO 80) |
| | | VLNSINVAVFSK |
| | | (SEQ ID NO 81) |
| | | VMLDYRDR |
| | | VYMDFLEDCCSR ox cam cam |
| | | (SEQ ID NO 83) |
| gi\|11277085 | Albumin (fragment) | AETFTFHSDICTLPEKEK cam |
| | | (SEQ ID NO 84) |
| | | APQVSTPTLVEAAR |
| | | (SEQ ID NO 85) |
| | | CCAEANPPACYGTVLAEFQPLVEEPK cam cam cam |
| | | (SEQ ID NO 86) |
| | | CCAEANPPACYGTVLAEFQPLVEEPKNLVK cam |
| | | cam cam |
| | | (SEQ ID NO 87) |
| | | CCSGSLVER cam cam |
| | | (SEQ ID NO 88) |
| | | CCTLPEDQRLPCVEDYLSAILNR cam cam cam |
| | | (SEQ ID NO 89) |
| | | DVFLGTFLYEYSR |
| | | (SEQ ID NO 90) |
| | | ENPTTFMGHYLHEVAR |
| | | (SEQ ID NO 91) |
| | | HPDYSVSLLLR |
| | | (SEQ ID NO 92) |
| | | LGEYGFQNAILVR |
| | | (SEQ ID NO 93) |
| | | LPCVEDYLSAILNR cam |
| | | (SEQ ID NO 94) |
| | | LQTCCDKPLLK cam cam |
| | | (SEQ ID NO 95) |
| | | LQTCCDKPLLKK cam cam |
| | | (SEQ ID NO 96) |
| | | LSQTFPNADFAEITK |
| | | (SEQ ID NO 97) |
| | | LSQTFPNADFAEITKLATDLTK |
| | | (SEQ ID NO 98) |
| | | NYAEAKDVFLGTFLYEYSR |
| | | (SEQ ID NO 99) |
| | | QEPERNECFLQHK cam |
| | | (SEQ ID NO 100) |
| | | RHPDYSVSLLLR |
| | | (SEQ ID NO 101) |
| | | RPCFSALTVDETYVPK cam |
| | | (SEQ ID NO 102) |
| | | TNCDLYEKLGEYGFQNAILVR cam |
| | | (SEQ ID NO 103) |
| | | VCLLHEKTPVSEHVTK cam |
| | | (SEQ ID NO 104) |
| | | YMCENQATISSK cam |
| | | (SEQ ID NO 105) |
| | | YTQKAPQVSTPTLVEAAR |
| | | (SEQ ID NO 106) |
| gi\|33859506 | Albumin 1 | SEIAHRYNDLGEQHFK |
| | | (SEQ ID NO 107) |
| | | AETFTFHSDICTLPEKEK cam |
| | | (SEQ ID NO 108) |
| | | AFKAWAVAR |
| | | (SEQ ID NO 109) |
| | | AHCLSEVEHDTMPADLPAIAADFVEDQEVCK cam ox |
| | | cam |
| | | (SEQ ID NO 110) |
| | | APQVSTPTLVEAAR |
| | | (SEQ ID NO 111) |
| | | APQVSTPTLVEAARNLGR |
| | | (SEQ ID NO 112) |
| | | CCAEANPPACYGTVLAEFQPLVEEPK cam cam cam |
| | | (SEQ ID NO 113) |
| | | CCSGSLVER cam cam |
| | | (SEQ ID NO 114) |
| | | CCTLPEDQR cam cam |
| | | (SEQ ID NO 115) |
| | | CCTLPEDQRLPCVEDYLSAILNR cam cam cam |
| | | (SEQ ID NO 116) |
| | | DDNPSLPPFERPEAEAMCTSFK ox cam |
| | | (SEQ ID NO 117) |
| | | DVFLGTFLYEYSR |
| | | (SEQ ID NO 118) |
| | | DVFLGTFLYEYSRR |
| | | (SEQ ID NO 119) |
| | | EAHKSEIAHR |
| | | (SEQ ID NO 120) |
| | | ECCHGDLLECADDRAELAK cam cam cam |
| | | (SEQ ID NO 121) |
| | | EFKAETFTFHSDICTLPEK cam |
| | | (SEQ ID NO 122) |
| | | ENPTTFMGHYLHEVAR ox |
| | | (SEQ ID NO 123) |
| | | ENYGELADCCTK cam cam |
| | | (SEQ ID NO 124) |
| | | HPDYSVSLLLR |
| | | (SEQ ID NO 125) |
| | | LCAIPNLRENYGELADCCTK cam cam cam |
| | | (SEQ ID NO 126) |
| | | LGEYGFQNAILVR |
| | | (SEQ ID NO 127) |
| | | LPCVEDYLSAILNR cam |
| | | (SEQ ID NO 128) |
| | | LQTCCDKPLLK cam cam |
| | | (SEQ ID NO 129) |
| | | LQTCCDKPLLKK cam cam |
| | | (SEQ ID NO 130) |
| | | LSQTFPNADFAEITK |
| | | (SEQ ID NO 131) |
| | | LVQEVTDFAK |
| | | (SEQ ID NO 132) |
| | | QEPERNECFLQHK cam |
| | | (SEQ ID NO 133) |
| | | RHPDYSVSLLLR |
| | | (SEQ ID NO 134) |
| | | RPCFSALTVDETYVPK cam |
| | | (SEQ ID NO 135) |
| | | RPCFSALTVDETYVPKEFK cam |
| | | (SEQ ID NO 136) |
| | | SLHTLFGDKLCAIPNLR cam |
| | | (SEQ ID NO 137) |
| | | TNCDLYEKLGEYGFQNAILVR cam |
| | | (SEQ ID NO 138) |
| | | TVMDDFAQFLDTCCK cam cam |
| | | (SEQ ID NO 139) |
| | | YMCENQATISSK ox cam |
| | | (SEQ ID NO 140) |
| | | YNDLGEQHFK |
| | | (SEQ ID NO 141) |
| | | YNDLGEQHFKGLVLIAFSQYLQK |
| | | (SEQ ID NO 142) |
| | | YTQKAPQVSTPTLVEAAR |
| | | (SEQ ID NO 143) |
| gi\|42542817 | Alpha fetoprotein | ALQTMKQELLINLVK |
| | | (SEQ ID NO 144) |
| | | AQDQEVCFTEEGPK cam |
| | | (SEQ ID NO 145) |
| | | AQDQEVCFTEEGPKLISK cam |
| | | (SEQ ID NO 146) |
| | | CSQSGNLPGCQDNLEEELQK cam cam |
| | | (SEQ ID NO 147) |
| | | CSQSGNLPGCQDNLEEELQKHIEESQALSK cam |
| | | cam |
| | | (SEQ ID NO 148) |
| | | DETYAPPPFSEDK |
| | | (SEQ ID NO 149) |
| | | EGSMLNEHVCSVIR cam |
| | | (SEQ ID NO 150) |
| | | ELREGSMLNEHVCSVIR ox cam |
| | | (SEQ ID NO 151) |
| | | FIYEVSRR |
| | | (SEQ ID NO 152) |
| | | LQNLFLIGYTR |
| | | (SEQ ID NO 153) |
| | | NPFMYAPAILSLAAQYDK ox |
| | | (SEQ ID NO 154) |
| | | NSGDGCLESQLSVFLDEICHETELSNK cam cam |
| | | (SEQ ID NO 155) |
| | | QKPELTEEQLAAVTADFSGLLEK |
| | | (SEQ ID NO 156) |
| | | QSCALYQTLGDYKLQNLFLIGYTR cam |
| | | (SEQ ID NO 157) |
| | | TAPASVPPFQFPEPAESCK cam |
| | | (SEQ ID NO 158) |
| | | TAPASVPPFQFPEPAESCKAHEENR cam |
| | | (SEQ ID NO 159) |
| | | THPNLPVSVILR |
| | | (SEQ ID NO 160) |
| | | VMTYICSQQNILSSK cam |
| | | (SEQ ID NO 161) |
| | | YGLSGCCSQSGVER cam cam |
| | | (SEQ ID NO 162) |
| gi\|191765 | Alpha fetoprotein | APQVSTPTLVEAAR |
| | | (SEQ ID NO 163) |
| | | CCSGSLVER cam cam |
| | | (SEQ ID NO 164) |
| | | CCTLPEDQRLPCVEDYLSAILNR cam cam cam |
| | | (SEQ ID NO 165) |
| | | DVFLGTFLYEYSR |
| | | (SEQ ID NO 166) |
| | | HPDYSVSLLLR |
| | | (SEQ ID NO 167) |
| | | LGEYGFQNAILVR |
| | | (SEQ ID NO 168) |
| | | LPCVEDYLSAILNR cam |
| | | (SEQ ID NO 169) |
| | | LQTCCDKPLLK cam cam |
| | | (SEQ ID NO 170) |
| | | LQTCCDKPLLKK cam cam |
| | | (SEQ ID NO 171) |
| | | LSQTFPNADFAEITK |
| | | (SEQ ID NO 172) |
| | | LSQTFPNADFAEITKLATDLTK |
| | | (SEQ ID NO 173) |
| | | NYAEAKDVFLGTFLYEYSR |
| | | (SEQ ID NO 174) |
| | | RHPDYSVSLLLR |
| | | (SEQ ID NO 175) |
| | | TNCDLYEKLGEYGFQNAILVR cam |
| | | (SEQ ID NO 176) |
| | | VCLLHEKTPVSEHVTK cam |
| | | (SEQ ID NO 177) |
| | | YMCENQATISSK ox cam |
| | | (SEQ ID NO 178) |
| | | YTQKAPQVSTPTLVEAAR |
| | | (SEQ ID NO 179) |
| gi\|191844 | Alpha-1 protease | AVLTIDETGTEAAAATVFEAVPMSMPPILR |
| | inhibitor 2 | (SEQ ID NO 180) |
| | | DQSPASHEIATNLGDFAISLYR |
| | | (SEQ ID NO 181) |
| | | FDHPFLFIIFEEHTQSPIFVGK |
| | | (SEQ ID NO 182) |
| | | FLEEAKNHYQAEVFSVNFAESEEAK |
| | | (SEQ ID NO 183) |
| | | IFNNGADLSGITEENAPLK |
| | | (SEQ ID NO 184) |
| | | IFNNGADLSGITEENAPLKLSK |
| | | (SEQ ID NO 185) |
| | | IVEAVKELDQDTVFALANYILFK |
| | | (SEQ ID NO 186) |
| | | KPFDPENTEEAEFHVDK |
| | | (SEQ ID NO 187) |
| | | KVINDFVEK |
| | | (SEQ ID NO 188) |
| | | LSISGDYNLK |
| | | (SEQ ID NO 189) |
| | | LSISGDYNLKTLMSPLGITR |
| | | (SEQ ID NO 190) |
| | | MQHLEQTLNK |
| | | (SEQ ID NO 191) |
| | | MQHLEQTLNKELISK |
| | | (SEQ ID NO 192) |
| | | NHYQAEVFSVNFAESEEAK |
| | | (SEQ ID NO 193) |
| | | NHYQAEVFSVNFAESEEAKK |
| | | (SEQ ID NO 194) |
| | | SFQHLLQTLNRPDSELQLSTGNGLFVNNDLK |
| | | (SEQ ID NO 195) |
| | | TLMSPLGITR |
| | | (SEQ ID NO 196) |
| | | VINDFVEKGTQGK |
| | | (SEQ ID NO 197) |
| | | WKKPFDPENTEEAEFHVDK |
| | | (SEQ ID NO 198) |
| gi\|31982171 | Alpha-2 | ADSHFRHGIPFFVK |
| | macroglobulin MUG1 | (SEQ ID NO 199) |
| | | AHFSVMGDILSSAIR |
| | | (SEQ ID NO 200) |
| | | ALMAYAFALAGNQNK |
| | | (SEQ ID NO 201) |
| | | ALMAYAFALAGNQNKR ox |
| | | (SEQ ID NO 202) |
| | | ALSCLESSWK cam |
| | | (SEQ ID NO 203) |
| | | DGSYSAFGDQNGER |
| | | (SEQ ID NO 204) |
| | | EGNTWLTAFVLK |
| | | (SEQ ID NO 205) |
| | | ESGEKEEHSFTVMEFVLPR |
| | | (SEQ ID NO 206) |
| | | ESVVFVQTDKPVYKPGQSVK |
| | | (SEQ ID NO 207) |
| | | FALEIPVEFSMVPMAK ox ox |
| | | (SEQ ID NO 208) |
| | | FSTSQSLPASQTR |
| | | (SEQ ID NO 209) |
| | | HTNLVPHGTEKDVYR |
| | | (SEQ ID NO 210) |
| | | HTSSWLVTPK |
| | | (SEQ ID NO 211) |
| | | HVAYAVYSLSK |
| | | (SEQ ID NO 212) |
| | | LPIICFDYGMVPISAPR cam ox |
| | | (SEQ ID NO 213) |
| | | LQVTASPQSLCGLR cam |
| | | (SEQ ID NO 214) |
| | | LSEDQEDCILYSSWLAEK cam |
| | | (SEQ ID NO 215) |
| | | LSSSDEEDFLYVDIKGPTHEFSK |
| | | (SEQ ID NO 216) |
| | | LVDIKGDPIPNEK |
| | | (SEQ ID NO 217) |
| | | MLRPLNELLPLAYIEDPKK |
| | | (SEQ ID NO 218) |
| | | MLSGFIPLKPTVK |
| | | (SEQ ID NO 219) |
| | | MSLVLPPTVVK |
| | | (SEQ ID NO 220) |
| | | MSLVLPPTVVKDSAR |
| | | (SEQ ID NO 221) |
| | | NKESVVFVQTDKPVYKPGQSVK |
| | | (SEQ ID NO 222) |
| | | NLFDELVLDKDLFQCVSFIIPR cam |
| | | (SEQ ID NO 223) |
| | | QMSFSLAAEPIQGPYK ox |
| | | (SEQ ID NO 224) |
| | | QQNSNGGFSSTQDTVVALDALSK |
| | | (SEQ ID NO 225) |
| | | SLDEEAIKENNSIHWK |
| | | (SEQ ID NO 226) |
| | | SQKTTLVTIQSTGSFSQK |
| | | (SEQ ID NO 227) |
| | | TTLVTIQSTGSFSQK |
| | | (SEQ ID NO 228) |
| | | YTYGKPVPGHVK |
| | | (SEQ ID NO 229) |
| gi\|7304875 | Alpha-2-HS- | ANLMHNLGGEEVSVACK cam |
| | glycoprotein (Fetuin) | (SEQ ID NO 230) |
| | | AQNVPLPVSTLVEFVIAATDCTAK cam |
| | | (SEQ ID NO 231) |
| | | CNLLAEK |
| | | (SEQ ID NO 232) |
| | | CNLLAEKQHGFCK cam cam |
| | | (SEQ ID NO 233) |
| | | ELACDDPEAEQVALLAVDYLNNHLLQGFK cam |
| | | (SEQ ID NO 234) |
| | | EVTDPAKCNLLAEK cam |
| | | (SEQ ID NO 235) |
| | | HAFSPVASVESASGETLHSPK |
| | | (SEQ ID NO 236) |
| | | QLTEHAVEGDCDFHILK cam |
| | | (SEQ ID NO 237) |
| | | QLTEHAVEGDCDFHILKQDGQFR cam |
| | | (SEQ ID NO 238) |
| | | QVLNQIDK |
| | | (SEQ ID NO 239) |
| | | QVLNQIDKVK |
| | | (SEQ ID NO 240) |
| | | TYHDLR |
| | | (SEQ ID NO 241) |
| | | VGQPGAAGPVSPMCPGR cam |
| | | (SEQ ID NO 242) |
| gi\|14318646 | Amylase 1, salivary | AHFSISNSAEDPFIAIHAESKI |
| | | (SEQ ID NO 243) |
| | | AILDKLHNLNTK |
| | | (SEQ ID NO 244) |
| | | ALVFVDNHDNQR |
| | | (SEQ ID NO 245) |
| | | DFPGVPYSGFDFNDGK |
| | | (SEQ ID NO 246) |
| | | GHGAGGASILTFWDAR |
| | | (SEQ ID NO 247) |
| | | LSGLLDLALEKDYVR |
| | | (SEQ ID NO 248) |
| | | NWGEGWGLMPSDR ox |
| | | (SEQ ID NO 249) |
| | | SGNEDEFRDMVNR |
| | | (SEQ ID NO 250) |
| | | TAIVHLFEWR |
| | | (SEQ ID NO 251) |
| | | TASGGIENYQDAAQVR |
| | | (SEQ ID NO 252) |
| | | VADYMNHLIDIGVAGFR ox |
| | | (SEQ ID NO 253) |
| | | VMSSYYWPR |
| | | (SEQ ID NO 254) |
| | | WVDIAKECER cam |
| | | (SEQ ID NO 255) |
| gi\|425520 | Anti-colorectal | ADAAPTVSIFPPSSEQLTSGGASVVCFLNNFYPK |
| | carcinoma light chain | cam |
| | | (SEQ ID NO 256) |
| | | ASQDINSYLSWFQQKPGK |
| | | (SEQ ID NO 257) |
| | | HNSYTCEATHK cam |
| | | (SEQ ID NO 258) |
| | | MTQSPSSMYASLGER ox |
| | | (SEQ ID NO 259) |
| | | RADAAPTVSIFPPSSEQLTSGGASVVCFLNNFYPK |
| | | cam |
| | | (SEQ ID NO 260) |
| | | TPAQFLGILLLWFPGMK ox |
| | | (SEQ ID NO 261) |
| gi\|26345182 | Apolipoprotein A1 | ARPALEDLR |
| | (Unnamed protein | (SEQ ID NO 262) |
| | product) | DFANVYVDAVKDSGR |
| | | (SEQ ID NO 263) |
| | | DFWDNLEKETDWVR |
| | | (SEQ ID NO 264) |
| | | HSLMPMLETLK ox |
| | | (SEQ ID NO 265) |
| | | LAELKSNPTLNEYHTR |
| | | (SEQ ID NO 266) |
| | | LSPVAEEFRDR |
| | | (SEQ ID NO 267) |
| | | MRTHVDSLR |
| | | (SEQ ID NO 268) |
| | | QKLQELQGR |
| | | (SEQ ID NO 269) |
| | | QKVQPYLDEFQK |
| | | (SEQ ID NO 270) |
| | | SNPTLNEYHTR |
| | | (SEQ ID NO 271) |
| | | TQLAPHSEQMR |
| | | (SEQ ID NO 272) |
| | | TQLAPHSEQMRESLAQR ox |
| | | (SEQ ID NO 273) |
| | | TQVQSVIDKASETLTAQ |
| | | (SEQ ID NO 274) |
| | | VAPLGAELQESARQK |
| | | (SEQ ID NO 275) |
| | | VKDFANVYVDAVK |
| | | (SEQ ID NO 276) |
| | | VQPYLDEFQK |
| | | (SEQ ID NO 277) |
| | | VQPYLDEFQKK |
| | | (SEQ ID NO 278) |
| | | WKEDVELYR |
| | | (SEQ ID NO 279) |
| gi\|14789706 | Apolipoprotein A4 | ALVQQLEQFR |
| | | (SEQ ID NO 280) |
| | | ALVQQLEQFRQQLGPNSGEVESHLSFLEK |
| | | (SEQ ID NO 281) |
| | | ATIDQNLEDLRR |
| | | (SEQ ID NO 282) |
| | | EAVEQFQKTDVTQQLSTLFQDK |
| | | (SEQ ID NO 283) |
| | | EKVNSFMSTLEK |
| | | (SEQ ID NO 284) |
| | | FLKAAVLTLALVAITGTR |
| | | (SEQ ID NO 285) |
| | | GSPDQPQALPLPEQAQEQAQEQAQEQVQPKPLES |
| | | (SEQ ID NO 286) |
| | | LGDASTYADGVHNK |
| | | (SEQ ID NO 287) |
| | | LNHQMEGLAFQMK |
| | | (SEQ ID NO 288) |
| | | LNHQMEGLAFQMKK |
| | | (SEQ ID NO 289) |
| | | LQEHLKPYAVDLQDQINTQTQEMK |
| | | (SEQ ID NO 290) |
| | | LQLTPYIQR |
| | | (SEQ ID NO 291) |
| | | LVPFVVQLSGHLAQETER |
| | | (SEQ ID NO 292) |
| | | MMPHANKVTQTFGENMQK ox |
| | | (SEQ ID NO 293) |
| | | NLAPLVEDVQSK |
| | | (SEQ ID NO 294) |
| | | NMEELKGHLTPR |
| | | (SEQ ID NO 295) |
| | | QLEQQVEEFRR |
| | | (SEQ ID NO 296) |
| | | QQLGPNSGEVESHLSFLEK |
| | | (SEQ ID NO 297) |
| | | SLAPLTVGVQEK |
| | | (SEQ ID NO 298) |
| | | SLAPLTVGVQEKLNHQMEGLAFQMK |
| | | (SEQ ID NO 299) |
| | | TDVTQQLSTLFQDK |
| | | (SEQ ID NO 300) |
| | | TDVTQQLSTLFQDKLGDASTYADGVHNK |
| | | (SEQ ID NO 301 |
| | | TVEPMGEMFNK |
| | | (SEQ ID NO 302) |
| | | VKGNTEGLQK |
| | | (SEQ ID NO 303) |
| | | VNSFMSTLEK |
| | | (SEQ ID NO 304) |
| gi\|6753096 | Apolipoprotein A-I | AQSVIDKASETLTAQ |
| | | (SEQ ID NO 305) |
| | | ARPALEDLR |
| | | (SEQ ID NO 306) |
| | | DFANVYVDAVK |
| | | (SEQ ID NO 307) |
| | | DFWDNLEKETDWVR |
| | | (SEQ ID NO 308) |
| | | HSLMPMLETLK ox ox |
| | | (SEQ ID NO 309) |
| | | LAELKSNPTLNEYHTR |
| | | (SEQ ID NO 310) |
| | | LQELQGR |
| | | (SEQ ID NO 311) |
| | | LSPVAEEFR |
| | | (SEQ ID NO 312) |
| | | LSPVAEEFRDR |
| | | (SEQ ID NO 313) |
| | | MRTHVDSLR ox |
| | | (SEQ ID NO 314) |
| | | QEMNKDLEEVK ox |
| | | (SEQ ID NO 315) |
| | | QKLQELQGR |
| | | (SEQ ID NO 316) |
| | | THVDSLR |
| | | (SEQ ID NO 317) |
| | | TQLAPHSEQMR |
| | | (SEQ ID NO 318) |
| | | TQLAPHSEQMRESLAQR ox |
| | | (SEQ ID NO 319) |
| | | VAPLGAELQESARQK |
| | | (SEQ ID NO 320) |
| | | VKDFANVYVDAVK |
| | | (SEQ ID NO 321) |
| | | VQPYLDEFQK |
| | | (SEQ ID NO 322) |
| | | VQPYLDEFQKK |
| | | (SEQ ID NO 323) |
| | | WKEDVELYR |
| | | (SEQ ID NO 324) |
| gi\|6753102 | Apolipoprotein E | AQAFGDRIR |
| | | (SEQ ID NO 325) |
| | | EHMEEVRSK |
| | | (SEQ ID NO 326) |
| | | ELEEQLGPVAEETR |
| | | (SEQ ID NO 327) |
| | | ELEEQLGPVAEETRAR |
| | | (SEQ ID NO 328) |
| | | ERLGPLVEQGR |
| | | (SEQ ID NO 329) |
| | | GRLEEVGNQAR |
| | | (SEQ ID NO 330) |
| | | GWFEPIVEDMHR ox |
| | | (SEQ ID NO 331) |
| | | LGADMEDLRNR |
| | | (SEQ ID NO 332) |
| | | LGKEVQAAQAR |
| | | (SEQ ID NO 333) |
| | | LGPLVEQGRQR |
| | | (SEQ ID NO 334) |
| | | LGQYRNEVHTMLGQSTEEIR |
| | | (SEQ ID NO 335) |
| | | LKGWFEPIVEDMHR |
| | | (SEQ ID NO 336) |
| | | LQAEIFQAR |
| | | (SEQ ID NO 337) |
| | | MEEQTQQIR |
| | | (SEQ ID NO 338) |
| | | NEVHTMLGQSTEEIRAR ox |
| | | (SEQ ID NO 339) |
| | | QWANLMEK |
| | | (SEQ ID NO 340) |
| | | SKMEEQTQQIR |
| | | (SEQ ID NO 341) |
| | | TANLGAGAAQPLRDR |
| | | (SEQ ID NO 342) |
| gi\|1938223 | Apolipoprotein H | ATFGCHETYKLDGPEEAECTK cam cam |
| | (beta-2 glycoprotein I) | (SEQ ID NO 343) |
| | | ATVLYQGMR ox |
| | | (SEQ ID NO 344) |
| | | CLPHFAMIGNDTVMCTEQGNWTR ox cam cam |
| | | (SEQ ID NO 345) |
| | | CPFPPRPENGYVNYPAKPVLLYK cam |
| | | (SEQ ID NO 346) |
| | | CPFPPRPENGYVNYPAKPVLLYKDK cam |
| | | (SEQ ID NO 347) |
| | | CSYTVEAHCR cam cam |
| | | (SEQ ID NO 348) |
| | | DGTIEIPSCFKEHSSLAFWK cam |
| | | (SEQ ID NO 349) |
| | | FTCPLTGMWPINTLR cam |
| | | (SEQ ID NO 350) |
| | | ITCPPPPVPK cam |
| | | (SEQ ID NO 351) |
| | | KATVLYQGMR |
| | | (SEQ ID NO 352) |
| | | KCSYTVEAHCR cam cam |
| | | (SEQ ID NO 353) |
| | | RFTCPLTGMWPINTLR cam |
| | | (SEQ ID NO 354) |
| | | TGTWSFLPTCR cam |
| | | (SEQ ID NO 355) |
| | | TSYDPGEQIVYSCKPGYVSR cam |
| | | (SEQ ID NO 356) |
| | | VCPFAGILENGIVR cam |
| | | (SEQ ID NO 357) |
| | | WSPDIPACAR cam |
| | | (SEQ ID NO 358) |
| gi\|9055162 | Apolipoprotein M | CVEEFQSLTSCLDFK cam cam |
| | | (SEQ ID NO 359) |
| | | CVEEFQSLTSCLDFKAFLVTPR cam cam |
| | | (SEQ ID NO 360) |
| | | FLLYNRSPHPPEK |
| | | (SEQ ID NO 361) |
| | | LTEGKGNMELR |
| | | (SEQ ID NO 362) |
| | | QLQLRATIR |
| | | (SEQ ID NO 363) |
| | | SPHPPEKCVEEFQSLTSCLDFK cam cam |
| | | (SEQ ID NO 364) |
| | | TDLFSSSCPGGIMLK cam ox |
| | | (SEQ ID NO 365) |
| | | TDLFSSSCPGGIMLKETGQGYQR cam |
| | | (SEQ ID NO 366) |
| | | TEGRPDMKTDLFSSSCPGGIMLK ox cam |
| | | (SEQ ID NO 367) |
| gi\|2921308 | Carboxylesterase | DAGVSTYMYEFR |
| | precursor | (SEQ ID NO 368) |
| | | FAPPQPAEPWSFVK |
| | | (SEQ ID NO 369) |
| | | MNEETASLLLR |
| | | (SEQ ID NO 370) |
| | | MNEETASLLLR ox |
| | | (SEQ ID NO 371) |
| | | MNEETASLLLRR ox |
| | | (SEQ ID NO 372) |
| | | MVMKFWANFAR ox ox |
| | | (SEQ ID NO 373) |
| | | RFHSELNISESMIPAVIEK ox |
| | | (SEQ ID NO 374) |
| | | SELILDMFGDIFFGIPAVLMSR ox ox |
| | | (SEQ ID NO 375) |
| | | SFNTVPYIVGFNK |
| | | (SEQ ID NO 376) |
| | | SLRDAGVSTYMYEFR |
| | | (SEQ ID NO 377) |
| | | VLGKYISLEGFEQPVAVFLGVPFAKPPLGSLR |
| | | (SEQ ID NO 378) |
| | | YISLEGFEQPVAVFLGVPFAKPPLGSLR |
| | | (SEQ ID NO 379) |
| | | YRPSFVSDK |
| | | (SEQ ID NO 380) |
| gi\|49523333 | Clusterin | ASGIIDTLFQDR |
| | (Apolipoprotein J) | (SEQ ID NO 381) |
| | | ASGIIDTLFQDRFFAR |
| | | (SEQ ID NO 382) |
| | | ELHDPHYFSPIGFPHK |
| | | (SEQ ID NO 383) |
| | | ELHDPHYFSPIGFPHKRPHFLYPK |
| | | (SEQ ID NO 384) |
| | | HTCMKFYAR cam ox |
| | | (SEQ ID NO 385) |
| | | QQSQVLDAMQDSFAR ox |
| | | (SEQ ID NO 386) |
| | | RPHFLYPK |
| | | (SEQ ID NO 387) |
| | | SLLNSLEEAKK |
| | | (SEQ ID NO 388) |
| | | TLIEKTNAER |
| | | (SEQ ID NO 389) |
| | | YINKEIQNAVQGVK |
| | | (SEQ ID NO 390) |
| gi\|6753798 | Coagulation factor II | DNLSPPLGQCLTER cam |
| | (prothrombin) | (SEQ ID NO 391) |
| | | DTTEKELLDSYIDGR |
| | | (SEQ ID NO 392) |
| | | EECSVPVCGQEGR cam cam |
| | | (SEQ ID NO 393) |
| | | ENLDRDIALLK |
| | | (SEQ ID NO 394) |
| | | ETFMDCLEGR cam |
| | | (SEQ ID NO 395) |
| | | ETFMDCLEGR ox cam |
| | | (SEQ ID NO 396) |
| | | GIAPWQVMLFR |
| | | (SEQ ID NO 397) |
| | | GIAPWQVMLFRK ox |
| | | (SEQ ID NO 398) |
| | | GKYGFYTHVFR |
| | | (SEQ ID NO 399) |
| | | IRITDNMFCAGFK cam |
| | | (SEQ ID NO 400) |
| | | ITDNMFCAGFK cam |
| | | (SEQ ID NO 401) |
| | | KPVPFSDYIHPVCLPDKQTVTSLLR cam |
| | | (SEQ ID NO 402) |
| | | LYQGNLAVTTLGSPCLPWNSLPAK cam |
| | | (SEQ ID NO 403) |
| | | NPDSSTTGPWCYTTDPTVR cam |
| | | (SEQ ID NO 404) |
| | | REECSVPVCGQEGR cam cam |
| | | (SEQ ID NO 405) |
| | | RGDACEGDSGGPFVMK cam |
| | | (SEQ ID NO 406) |
| | | SGGSKDNLSPPLGQCLTER cam |
| | | (SEQ ID NO 407) |
| | | SPQELLCGASLISDR cam |
| | | (SEQ ID NO 408) |
| | | TFGLGEADCGLRPLFEK cam |
| | | (SEQ ID NO 409) |
| | | TLSKYQDFDPEVK |
| | | (SEQ ID NO 410) |
| | | TTDAEFHTFFNEK |
| | | (SEQ ID NO 411) |
| | | WYQMGIVSWGEGCDRK cam |
| | | (SEQ ID NO 412) |
| | | YNWRENLDR |
| | | (SEQ ID NO 413) |
| | | YTVCDSVRKPR cam |
| | | (SEQ ID NO 414) |
| gi\|13624321 | Coagulation factor | CNEYYLLK cam |
| | XIII, beta subunit | (SEQ ID NO 415) |
| | | CNEYYLLKGSETSR cam |
| | | (SEQ ID NO 416) |
| | | CTAEGWSPNPR cam |
| | | (SEQ ID NO 417) |
| | | DAYISETSIAGSVLR |
| | | (SEQ ID NO 418) |
| | | DIVAYTCTAGYYTTTGK cam |
| | | (SEQ ID NO 419) |
| | | GDVRYPMCIR cam |
| | | (SEQ ID NO 420) |
| | | GMCASPPVIR cam |
| | | (SEQ ID NO 421) |
| | | IAQYYYTFK |
| | | (SEQ ID NO 422) |
| | | ILHGDLIDFVCK cam |
| | | (SEQ ID NO 423) |
| | | LIENGYFHPVK |
| | | (SEQ ID NO 424) |
| | | LSFFCLAGYATESGKQEEQIR cam |
| | | (SEQ ID NO 425) |
| | | QCDFPTVENGR cam |
| | | (SEQ ID NO 426) |
| | | QGYNLSPSIPLSEISAQCNR cam |
| | | (SEQ ID NO 427) |
| | | QTGEAECQANGWSLTPQCNK cam cam |
| | | (SEQ ID NO 428) |
| | | RDAYISETSIAGSVLR |
| | | (SEQ ID NO 429) |
| | | SFYFPMSVDK |
| | | (SEQ ID NO 430) |
| | | SFYFPMSVDKK ox |
| | | (SEQ ID NO 431) |
| | | TTGGKDEEVVHCLSAGWSSQPSCR cam cam |
| | | (SEQ ID NO 432) |
| | | VKDIVAYTCTAGYYTTTGK cam |
| | | (SEQ ID NO 433) |
| gi\|6996919 | Complement | AEGIPEFYDYDVALVK |
| | histocompatibility 2, | (SEQ ID NO 434) |
| | complement | ALLDIGRDPK |
| | component factor B | (SEQ ID NO 435) |
| | | ALRLPQTATCK cam |
| | | (SEQ ID NO 436) |
| | | CLTNLIEKVASYGVRPR cam |
| | | (SEQ ID NO 437) |
| | | CPRPQDFENGEFWPR cam |
| | | (SEQ ID NO 438) |
| | | DVKALFVSEQGK |
| | | (SEQ ID NO 439) |
| | | FIQVGVISWGVVDVCR cam |
| | | (SEQ ID NO 440) |
| | | GDSGGPLIVHKR |
| | | (SEQ ID NO 441) |
| | | GGSFQLLQGGQALEYLCPSGFYPYPVQTR cam |
| | | (SEQ ID NO 442) |
| | | GNDYHKQPWQAK |
| | | (SEQ ID NO 443) |
| | | HVIIIMTDGLHNMGGNPVTVIQDIR ox |
| | | (SEQ ID NO 444) |
| | | KDNEHHVFK |
| | | (SEQ ID NO 445) |
| | | LEDIVTYHCSR cam |
| | | (SEQ ID NO 446) |
| | | LKYGQTLRPICLPCTEGTTR cam cam |
| | | (SEQ ID NO 447) |
| | | LNQISYEDHKLK |
| | | (SEQ ID NO 448) |
| | | NPREDYLDVYVFGVGPLVDSVNINALASK |
| | | (SEQ ID NO 449) |
| | | QHKEQLLPVK |
| | | (SEQ ID NO 450) |
| | | QQLVPSYAR |
| | | (SEQ ID NO 451) |
| | | RDLEIEEVLFHPK |
| | | (SEQ ID NO 452) |
| | | RQQLVPSYAR |
| | | (SEQ ID NO 453) |
| | | SLSLCGMVWEHK cam |
| | | (SEQ ID NO 454) |
| | | SLSLCGMVWEHKK cam |
| | | (SEQ ID NO 455) |
| | | SRFIQVGVISWGVVDVCR cam |
| | | (SEQ ID NO 456) |
| | | STGSWSDLQTR |
| | | (SEQ ID NO 457) |
| | | VKDASEVVTPR |
| | | (SEQ ID NO 458) |
| | | VKDMEDLENVFYQMIDETK ox ox |
| | | (SEQ ID NO 459) |
| | | VSDERSSDADWVTEK |
| | | (SEQ ID NO 460) |
| | | WDGQTAICDDGAGYCPNPGIPIGTR cam cam |
| | | (SEQ ID NO 461) |
| | | YGLLTYATVPK |
| | | (SEQ ID NO 462) |
| | | YGQTLRPICLPCTEGTTR cam cam |
| | | (SEQ ID NO 463) |
| gi\|28175786 | Complement C3 | AAVFNHFISDGVKK |
| | precursor (HSE-MSF) | (SEQ ID NO 464) |
| | | AFYEHAPK |
| | | (SEQ ID NO 465) |
| | | AVMVSFQSGYLFIQTDK ox |
| | | (SEQ ID NO 466) |
| | | DNHLAPGQQTTLR |
| | | (SEQ ID NO 468) |
| | | DNHLAPGQQTTLRIEGNQGAR |
| | | (SEQ ID NO 469) |
| | | DSCIGTLVVKGDPR cam |
| | | (SEQ ID NO 470) |
| | | EPGQDLVVLSLPITPEFIPSFR |
| | | (SEQ ID NO 471) |
| | | EVVADSVWVDVKDSCIGTLVVK cam |
| | | (SEQ ID NO 472) |
| | | FFKPAMPFDLMVFVTNPDGSPASK ox ox |
| | | (SEQ ID NO 473) |
| | | GDPRDNHLAPGQQTTLR |
| | | (SEQ ID NO 474) |
| | | IEGNQGAR |
| | | (SEQ ID NO 475) |
| | | IFTVDNNLLPVGK |
| | | (SEQ ID NO 476) |
| | | IRAFYEHAPK |
| | | (SEQ ID NO 477) |
| | | IRYYTYLVMNK ox |
| | | (SEQ ID NO 478) |
| | | ISLAHSLTR |
| | | (SEQ ID NO 479) |
| | | KVLMEGVRPSNADALVGK |
| | | (SEQ ID NO 480) |
| | | LESEETIVLEAHDAQGDIPVTVTVQDFLKR |
| | | (SEQ ID NO 481) |
| | | LSINTPNSR |
| | | (SEQ ID NO 482) |
| | | LVAYYTLIGASGQR |
| | | (SEQ ID NO 483) |
| | | MELKPGDNLNVNFHLR ox |
| | | (SEQ ID NO 484) |
| | | NVDGTAFVIFGVQDGDKK |
| | | (SEQ ID NO 485) |
| | | NYAGVFMDAGLAFK |
| | | (SEQ ID NO 486) |
| | | QIFSAEFEVK |
| | | (SEQ ID NO 487) |
| | | QPLTITVR |
| | | (SEQ ID NO 488) |
| | | QVLTSEKTVLTGASGHLR |
| | | (SEQ ID NO 489) |
| | | SGIPIVTSPYQIHFTK |
| | | (SEQ ID NO 490) |
| | | SLYVSVTVILHSGSDMVEAER ox |
| | | (SEQ ID NO 491) |
| | | TIYTPGSTVLYR |
| | | (SEQ ID NO 492) |
| | | TMEAHPYSTMHNSNNYLHLSVSR ox ox |
| | | (SEQ ID NO 493) |
| | | TSQGLQTEQR |
| | | (SEQ ID NO 494) |
| | | TVLTGASGHLR |
| | | (SEQ ID NO 495) |
| | | TVVILIETPDGIPVKR |
| | | (SEQ ID NO 496) |
| | | VEPTETFYYIDDPNGLEVSIIAK |
| | | (SEQ ID NO 497) |
| | | VGLVAVDKGVFVLNK |
| | | (SEQ ID NO 498) |
| | | VLMEGVRPSNADALVGK |
| | | (SEQ ID NO 499) |
| | | VVIEDGVGDAVLTR |
| | | (SEQ ID NO 500) |
| | | VVIEDGVGDAVLTRK |
| | | (SEQ ID NO 501) |
| | | YVTVVANFGETVVEK |
| | | (SEQ ID NO 502) |
| | | YYTYLVMNK |
| | | (SEQ ID NO 503) |
| gi\|220349 | Complement C4 | APHIQLVAQSPWLR |
| | | (SEQ ID NO 504) |
| | | ATETQGVNLLFSSR |
| | | (SEQ ID NO 505) |
| | | ATETQGVNLLFSSRR |
| | | (SEQ ID NO 506) |
| | | FALMDEQGKR |
| | | (SEQ ID NO 507) |
| | | FVSSAFSLDLSR |
| | | (SEQ ID NO 508) |
| | | GHIFVQTDQPIYNPGQR |
| | | (SEQ ID NO 509) |
| | | GLETQAKLVEGR |
| | | (SEQ ID NO 510) |
| | | GTGFLSIEPLDPR |
| | | (SEQ ID NO 511) |
| | | HLVPGAHFLLQALVQEMSGSEASNVPVK ox |
| | | (SEQ ID NO 512) |
| | | KYVLPNFEVK |
| | | (SEQ ID NO 513) |
| | | LLLFSPSVVNLGTPLSVGVQLLDAPPGQEVK |
| | | (SEQ ID NO 514) |
| | | LLVSAGSLYPAIAR |
| | | (SEQ ID NO 515) |
| | | LSSGDDFVLLSLEVPLEDVR |
| | | (SEQ ID NO 516) |
| | | LTVQAPPSRGTGFLSIEPLDPR |
| | | (SEQ ID NO 517) |
| | | MRPSTDFLTITVENSHGLR ox |
| | | (SEQ ID NO 518) |
| | | NTAFKATETQGVNLLFSSR |
| | | (SEQ ID NO 519) |
| | | RAPHIQLVAQSPWLR |
| | | (SEQ ID NO 520) |
| | | RGHIFVQTDQPIYNPGQR |
| | | (SEQ ID NO 521) |
| | | SCGLFDLRR cam |
| | | (SEQ ID NO 522) |
| | | TFLRGLETQAK |
| | | (SEQ ID NO 523) |
| | | YIYGKPVQGVAYTR |
| | | (SEQ ID NO 524) |
| | | YVLPNFEVK |
| | | (SEQ ID NO 525) |
| gi\|309119 | Complement C4b- | ATYTHRDSVR |
| | binding protein | (SEQ ID NO 526) |
| | precursor | GKGVAWSNPFPECVIVK cam |
| | | (SEQ ID NO 527) |
| | | HSGTEDFYPYNHGISYTCDPGFR cam |
| | | (SEQ ID NO 528) |
| | | IICSQPNILHGVIVSGYK cam |
| | | (SEQ ID NO 529) |
| | | IICSQPNILHGVIVSGYKATYTHR cam |
| | | (SEQ ID NO 530) |
| | | ITLVTYECDKGYR cam |
| | | (SEQ ID NO 531) |
| | | MMVYCKPSGEWEISVSCAK ox cam cam |
| | | (SEQ ID NO 532) |
| | | TMQYVPNSHDVK ox |
| | | (SEQ ID NO 533) |
| | | TVPVWSSSPPTCEK cam |
| | | (SEQ ID NO 534) |
| | | WKGTAPQCK cam |
| | | (SEQ ID NO 535) |
| | | YECLPGYGR cam |
| | | (SEQ ID NO 536) |
| gi\|15030019 | Complement | ALVHLPLEYNSAVYSR |
| | component 6 | (SEQ ID NO 537) |
| | | ATDLQLSDVFLK |
| | | (SEQ ID NO 538) |
| | | CLPDRTWSQGDVECQR cam cam |
| | | (SEQ ID NO 539) |
| | | ECNNPAPQR cam |
| | | (SEQ ID NO 540) |
| | | FDPCQCAPCPNNGRPR cam cam cam |
| | | (SEQ ID NO 541) |
| | | FFPIPIFHFSEK |
| | | (SEQ ID NO 542) |
| | | FLCDSGRCIPSK cam cam |
| | | (SEQ ID NO 543) |
| | | FLYMEIHKEDTCTK cam |
| | | (SEQ ID NO 544) |
| | | GEVLDNSFTGGICK cam |
| | | (SEQ ID NO 545) |
| | | LSGTECLCVCQSGTYGENCER cam cam cam cam |
| | | (SEQ ID NO 546) |
| | | NEDSLSVDER |
| | | (SEQ ID NO 547) |
| | | NEHSHYSSAFNK |
| | | (SEQ ID NO 548) |
| | | NEHSHYSSAFNKVIK |
| | | (SEQ ID NO 549) |
| | | NIPCAVTKR cam |
| | | (SEQ ID NO 550) |
| | | NKFLCDSGR cam |
| | | (SEQ ID NO 551) |
| | | NKNEDSLSVDER |
| | | (SEQ ID NO 552) |
| | | QELQNSGLTEEEAQNCVQYETK cam |
| | | (SEQ ID NO 553) |
| | | QRQVVVNDYYWK |
| | | (SEQ ID NO 554) |
| | | QVVVNDYYWK |
| | | (SEQ ID NO 555) |
| | | SQQAAALAWEK |
| | | (SEQ ID NO 556) |
| | | SVLRPSQFGGQPCTEPLVTFQPCVPSK cam cam |
| | | (SEQ ID NO 557) |
| | | TKFFPIPIFHFSEK |
| | | (SEQ ID NO 558) |
| | | TRECNNPAPQR cam |
| | | (SEQ ID NO 559) |
| | | TSCLKPVVQDVLTISPFQR cam |
| | | (SEQ ID NO 560) |
| | | TWSQGDVECQR cam |
| | | (SEQ ID NO 561) |
| | | VYQIGESIELTCPR cam |
| | | (SEQ ID NO 562) |
| | | YGGSFLQGSEK |
| | | (SEQ ID NO 563) |
| gi\|27462724 | Complement | CIPACGVPTEPFQVHQR cam cam |
| | component C1SA | (SEQ ID NO 564) |
| | | IFGGQPAKIENFPWQVFFNHPR |
| | | (SEQ ID NO 565) |
| | | ISDPLMYVGTMSVR ox ox |
| | | (SEQ ID NO 566) |
| | | LQVVFTSDFSNEER |
| | | (SEQ ID NO 567) |
| | | LRYHGDPISCAK cam |
| | | (SEQ ID NO 568) |
| | | NQQFGPYCGNGFPGPLTIR cam |
| | | (SEQ ID NO 569) |
| | | SPNSPIIEEFQFPYNK |
| | | (SEQ ID NO 570) |
| | | WVNDQLGIELPR |
| | | (SEQ ID NO 571) |
| | | YHGDPISCAK cam |
| | | (SEQ ID NO 572) |
| gi\|19072788 | Complement | CDNGFSPPSGYSWDYLR cam |
| | component factor h | (SEQ ID NO 573) |
| | | CIEKIPCSQPPTIEHGSINLPR cam cam |
| | | (SEQ ID NO 574) |
| | | CLPVTELENGR cam |
| | | (SEQ ID NO 575) |
| | | CSPPYILNGIYTPHR cam |
| | | (SEQ ID NO 576) |
| | | CTAQGWEPEVPCVR cam cam |
| | | (SEQ ID NO 577) |
| | | CTAQGWEPEVPCVRK cam cam |
| | | (SEQ ID NO 578) |
| | | CTLKPCEFPQFK cam cam |
| | | (SEQ ID NO 579) |
| | | CTLKPCEFPQFKYGR cam cam |
| | | (SEQ ID NO 580) |
| | | CTPTGWIPVPR cam |
| | | (SEQ ID NO 581) |
| | | CVATDQLEKCR cam cam |
| | | (SEQ ID NO 582) |
| | | CVEILCTPPR cam cam |
| | | (SEQ ID NO 583) |
| | | GDAVCTGSGWSSQPFCEEKR cam cam |
| | | (SEQ ID NO 584) |
| | | HTEKIYSHSGEDIEFGCK cam |
| | | (SEQ ID NO 585) |
| | | IIHRSDDEIR |
| | | (SEQ ID NO 586) |
| | | IKTCSASDIHIDNGFLSESSSIYALNR cam |
| | | (SEQ ID NO 587) |
| | | IPCSQPPTIEHGSINLPR cam |
| | | (SEQ ID NO 588) |
| | | IVSGAAETDQEYYFGQVVR |
| | | (SEQ ID NO 589) |
| | | KPCGHPGDTPFGSFR cam |
| | | (SEQ ID NO 590) |
| | | KVEYSHGEVVK |
| | | (SEQ ID NO 591) |
| | | LYYEESLRPNFPVSIGNK |
| | | (SEQ ID NO 592) |
| | | LYYEESLRPNFPVSIGNKYSYK |
| | | (SEQ ID NO 593) |
| | | NGKWVASNPSR |
| | | (SEQ ID NO 594) |
| | | SCDMPVFENSITKNTR cam ox |
| | | (SEQ ID NO 595) |
| | | SDDEIRYECNYGFYPVTGSTVSK cam |
| | | (SEQ ID NO 596) |
| | | SYRTGEQVTFR |
| | | (SEQ ID NO 597) |
| | | TCGDIPELEHGSAK cam |
| | | (SEQ ID NO 598) |
| | | TSCPPPPQIPNTQVIETTVK cam |
| | | (SEQ ID NO 599) |
| | | VGDLLEFSCHSGHR cam |
| | | (SEQ ID NO 600) |
| | | VGPDSVQCYHFGWSPGFPTCK cam cam |
| | | (SEQ ID NO 601) |
| | | YRVGDLLEFSCHSGHR cam |
| | | (SEQ ID NO 602) |
| gi\|6671744 | Complement | ECELPNSVPACVPWSPYLFQPNDR cam cam |
| | component factor i | (SEQ ID NO 603) |
| | | ITCGGIYIGGCWILTAAHCVRPSR cam cam cam |
| | | (SEQ ID NO 604) |
| | | SPSASDLPQEELVDQK |
| | | (SEQ ID NO 605) |
| | | VANYFDWISYHVGR |
| | | (SEQ ID NO 606) |
| | | VANYFDWISYHVGRSLVSQHNV |
| | | (SEQ ID NO 607) |
| | | VIGGKPANVGDYPWQVAIKDGQR |
| | | (SEQ ID NO 608) |
| | | VIVHEKYNGATFQNDIALIEMK |
| | | (SEQ ID NO 609) |
| gi\|9954973 | Complement D Chain | DFDSVPPVVR |
| | D, N-Terminally | (SEQ ID NO 610) |
| | Truncated C3dg | DICEGQVNSLPGSINK cam |
| | Fragment | (SEQ ID NO 611) |
| | | EADVSLTAFVLIALQEAR |
| | | (SEQ ID NO 612) |
| | | FLNTAKDR |
| | | (SEQ ID NO 613) |
| | | GYTQQLAFK |
| | | (SEQ ID NO 614) |
| | | KGYTQQLAFK |
| | | (SEQ ID NO 615) |
| | | QKPDGVFQEDGPVIHQEMIGGFR |
| | | (SEQ ID NO 616) |
| | | RQEALELIK |
| | | (SEQ ID NO 617) |
| | | WLILEK |
| | | (SEQ ID NO 618) |
| | | WLNDER |
| | | (SEQ ID NO 619) |
| gi\|6754132 | Complement | AKGNEQSFHVSLR |
| | histocompatibility 2, | (SEQ ID NO 620) |
| | Q region locus 10 | APWMEQEGPEYWER |
| | | (SEQ ID NO 621) |
| | | APWMEQEGPEYWERETQR |
| | | (SEQ ID NO 622) |
| | | AYLEAECVEWLLR cam |
| | | (SEQ ID NO 623) |
| | | FDSDAETPRMEPR |
| | | (SEQ ID NO 624) |
| | | FIIVGYVDDTQFVR |
| | | (SEQ ID NO 625) |
| | | FIIVGYVDDTQFVRFDSDAETPR |
| | | (SEQ ID NO 626) |
| | | GNEQSFHVSLR |
| | | (SEQ ID NO 627) |
| | | GYLQYAYDGR |
| | | (SEQ ID NO 628) |
| | | GYLQYAYDGRDYIALNEDLK |
| | | (SEQ ID NO 629) |
| | | KWEQAGAAEYYR |
| | | (SEQ ID NO 630) |
| | | TDPPKTHVTHHPGSEGDVTLR |
| | | (SEQ ID NO 631) |
| | | THVTHHPGSEGDVTLR |
| | | (SEQ ID NO 632) |
| | | TWTAADVAAIITR |
| | | (SEQ ID NO 633) |
| | | WASVVVPLGK |
| | | (SEQ ID NO 634) |
| | | WEQAGAAEYYR |
| | | (SEQ ID NO 635) |
| | | YFETSVSRPGLGEPR |
| | | (SEQ ID NO 636) |
| | | YLELGKETLLR |
| | | (SEQ ID NO 637) |
| gi\|54173 | Contraspin | ALYQTEAFTADFQQPTEAK |
| | | (SEQ ID NO 638) |
| | | AVLDVAETGTEAAAATGVIGGIR |
| | | (SEQ ID NO 639) |
| | | AVLDVAETGTEAAAATGVIGGIRK |
| | | (SEQ ID NO 640) |
| | | AVLPAVCFNRPFLIVIYHTSAQSILFMAK cam ox |
| | | (SEQ ID NO 641) |
| | | DLQILAEFHEK |
| | | (SEQ ID NO 642) |
| | | DLQILAEFHEKTR |
| | | (SEQ ID NO 643) |
| | | EVFTEQADLSGITEAK |
| | | (SEQ ID NO 644) |
| | | EVFTEQADLSGITEAKK |
| | | (SEQ ID NO 645) |
| | | FSIASDYRLEEDVLPEMGIK |
| | | (SEQ ID NO 646) |
| | | GKTMEEILEGLK ox |
| | | (SEQ ID NO 647) |
| | | HFRDEELSCSVLELK cam |
| | | (SEQ ID NO 648) |
| | | ISFDPQDTFESEFYLDEKR |
| | | (SEQ ID NO 649) |
| | | KLISELDDGTLMVLVNYIYFK ox |
| | | (SEQ ID NO 650) |
| | | KLSVSQVVHK |
| | | (SEQ ID NO 651) |
| | | KTLFSSQIEELNLPK |
| | | (SEQ ID NO 652) |
| | | LISELDDGTLMVLVNYIYFK ox |
| | | (SEQ ID NO 653) |
| | | MQQVEASLQPETLR |
| | | (SEQ ID NO 654) |
| | | MQQVEASLQPETLRK |
| | | (SEQ ID NO 655) |
| | | NIVFSPLSISAALALVSLGAK |
| | | (SEQ ID NO 656) |
| | | NQDKNIVFSPLSISAALALVSLGAK |
| | | (SEQ ID NO 657) |
| | | TLFSSQIEELNLPK |
| | | (SEQ ID NO 658) |
| | | TRALYQTEAFTADFQQPTEAK |
| | | (SEQ ID NO 659) |
| gi\|38614350 | Cp protein | AEDEHLGILGPPIHANVGDK |
| | | (SEQ ID NO 660) |
| | | AEDEHLGILGPPIHANVGDKVK |
| | | (SEQ ID NO 661) |
| | | AEVEDKVYVHLK |
| | | (SEQ ID NO 662) |
| | | AGLQAFFQVR |
| | | (SEQ ID NO 663) |
| | | ALYFEYTDGTFSK |
| | | (SEQ ID NO 664) |
| | | DCNKPSPEDNIQDR cam |
| | | (SEQ ID NO 665) |
| | | DIFTGLIGPMKICK cam |
| | | (SEQ ID NO 666) |
| | | DLYSGLIGPLIVCR cam |
| | | (SEQ ID NO 667) |
| | | DTANLFPHK |
| | | (SEQ ID NO 668) |
| | | EMGPTYADPVCLSK ox cam |
| | | (SEQ ID NO 669) |
| | | ETFTYEWTVPK |
| | | (SEQ ID NO 670) |
| | | EYTDGSFTNR |
| | | (SEQ ID NO 671) |
| | | EYTDGSFTNRK |
| | | (SEQ ID NO 672) |
| | | GQHPLSIQPMGVSFTAENEGTYYGPPGR ox |
| | | (SEQ ID NO 673) |
| | | IYHSHVDAPK |
| | | (SEQ ID NO 674) |
| | | IYHSHVDAPKDIASGLIGPLILCK cam |
| | | (SEQ ID NO 675) |
| | | IYTFHAHGVTYTK |
| | | (SEQ ID NO 676) |
| | | KALYFEYTDGTFSK |
| | | (SEQ ID NO 677) |
| | | KLISVDTEQSNFYLQNGPDR |
| | | (SEQ ID NO 678) |
| | | LISVDTEQSNFYLQNGPDR |
| | | (SEQ ID NO 679) |
| | | MAYREYTDGSFTNR ox |
| | | (SEQ ID NO 680) |
| | | MFGNLQGLTMHVK ox ox |
| | | (SEQ ID NO 681) |
| | | MFTTAPDQVDKEDEDFQESNK ox |
| | | (SEQ ID NO 682) |
| | | MYYSGVDPTKDIFTGLIGPMK |
| | | (SEQ ID NO 683) |
| | | NMATRPYSIHAHGVK |
| | | (SEQ ID NO 684) |
| | | NMATRPYSIHAHGVK ox |
| | | (SEQ ID NO 685) |
| | | QFEDFTVYLGER |
| | | (SEQ ID NO 686) |
| | | QKYTVNQCQR cam |
| | | (SEQ ID NO 687) |
| | | RAEDEHLGILGPPIHANVGDK |
| | | (SEQ ID NO 688) |
| | | RDTANLFPHK |
| | | (SEQ ID NO 689) |
| | | SGAGREDSACIPWAYYSTVDR cam |
| | | (SEQ ID NO 690) |
| | | SLTLLMNPDTK ox |
| | | (SEQ ID NO 691) |
| | | TESSTVVPTLPGEVR |
| | | (SEQ ID NO 692) |
| | | TIDKPAWLGFLGPVIK |
| | | (SEQ ID NO 693) |
| | | TYTWQIPER |
| | | (SEQ ID NO 694) |
| | | TYYVAAVEVEWDYSPSR |
| | | (SEQ ID NO 695) |
| | | VFFEQGATR |
| | | (SEQ ID NO 696) |
| | | VKDLYSGLIGPLIVCR cam |
| | | (SEQ ID NO 697) |
| | | VNKDNEEFLESNK |
| | | (SEQ ID NO 698) |
| | | YTVNQCQR cam |
| | | (SEQ ID NO 699) |
| gi\|19388017 | Cpn2 protein | AAHSQCAYSNPEGTVLLACEESR cam cam |
| | | (SEQ ID NO 700) |
| | | ALGLDEGEPAGSWDLTVEGR |
| | | (SEQ ID NO 701) |
| | | CRWLNIQLSSR cam |
| | | (SEQ ID NO 702) |
| | | DLRTLNLAQNLLTQLPK |
| | | (SEQ ID NO 703) |
| | | GAFQSLTGLQMLK |
| | | (SEQ ID NO 704) |
| | | GQLVPNLKQEQLICPVNPGHLSFR cam |
| | | (SEQ ID NO 705) |
| | | HLEPDAFGGLPR |
| | | (SEQ ID NO 706) |
| | | LFQSLRDLR |
| | | (SEQ ID NO 707) |
| | | LVSLTLSHNAITDLPEHVFR |
| | | (SEQ ID NO 708) |
| | | LVSLTLSHNAITDLPEHVFRNLEQLVK |
| | | (SEQ ID NO 709) |
| | | QEQLICPVNPGHLSFR cam |
| | | (SEQ ID NO 710) |
| | | TLNLAQNLLTQLPK |
| | | (SEQ ID NO 711) |
| | | TLPGRLFQSLR |
| | | (SEQ ID NO 712) |
| | | VVFLNTQVR |
| | | (SEQ ID NO 713) |
| | | WLNIQLSSR |
| | | (SEQ ID NO 714) |
| gi\|11055360 | Epidermal growth | ACGPDYYEVEEDGIR cam |
| | factor receptor | (SEQ ID NO 715) |
| | isoform 2 | ACGPDYYEVEEDGIRK cam |
| | | (SEQ ID NO 716) |
| | | AVNHVCNPLCSSEGCWGPEPR cam cam cam |
| | | (SEQ ID NO 717) |
| | | CNILEGEPR cam |
| | | (SEQ ID NO 718) |
| | | FSNNPILCNMDTIQWR cam |
| | | (SEQ ID NO 719) |
| | | GPDNCIQCAHYIDGPHCVK cam cam cam |
| | | (SEQ ID NO 720) |
| | | GRSPSDCCHNQCAAGCTGPR cam cam cam cam |
| | | (SEQ ID NO 721) |
| | | IICAQQCSHR cam cam |
| | | (SEQ ID NO 722) |
| | | IPLENLQIIR |
| | | (SEQ ID NO 723) |
| | | KLFGTPNQK |
| | | (SEQ ID NO 724) |
| | | LTQLGTFEDHFLSLQR |
| | | (SEQ ID NO 725) |
| | | MYNNCEVVLGNLEITYVQR ox cam |
| | | (SEQ ID NO 726) |
| | | NLCYANTINWK cam |
| | | (SEQ ID NO 727) |
| | | NLCYANTINWKK cam |
| | | (SEQ ID NO 728) |
| | | NLQEILIGAVR |
| | | (SEQ ID NO 729) |
| | | NYVVTDHGSCVR cam |
| | | (SEQ ID NO 730) |
| | | SLKEISDGDVIISGNR |
| | | (SEQ ID NO 731) |
| | | SPSDCCHNQCAAGCTGPR cam cam cam cam |
| | | (SEQ ID NO 732) |
| | | TIQEVAGYVLIALNTVER |
| | | (SEQ ID NO 733) |
| | | TPPLDPRELEILK |
| | | (SEQ ID NO 734) |
| | | YCTAISGDLHILPVAFKGDSFTR cam |
| | | (SEQ ID NO 735) |
| | | YSFGATCVKK cam |
| | | (SEQ ID NO 736) |
| gi\|6679689 | Esterase 1 | AISESGVVINTNVGKK |
| | | (SEQ ID NO 737) |
| | | FAPPQPAEPWSFVK |
| | | (SEQ ID NO 738) |
| | | ILSDMFSTEKEILPLK ox |
| | | (SEQ ID NO 739) |
| | | ISEDCLYLNIYSPADLTK cam |
| | | (SEQ ID NO 740) |
| | | KSELILDMFGDIFFGIPAVLLSR |
| | | (SEQ ID NO 741) |
| | | LKAEEVAFWTELLAK |
| | | (SEQ ID NO 742) |
| | | MNEETASLLLR |
| | | (SEQ ID NO 743) |
| | | MNEETASLLLRR |
| | | (SEQ ID NO 744) |
| | | MVMKFWANFAR |
| | | (SEQ ID NO 745) |
| | | NGNPNGEGLPHWPEYDEQEGYLQIGATTQQAQR |
| | | (SEQ ID NO 746) |
| | | QKTESELLEISGK |
| | | (SEQ ID NO 747) |
| | | RPQTVEGDHGDEIFFVFGAPLLK |
| | | (SEQ ID NO 748) |
| | | SELILDMFGDIFFGIPAVLLSR ox |
| | | (SEQ ID NO 749) |
| | | SFNTVPYIVGFNK |
| | | (SEQ ID NO 750) |
| | | SLRDAGVSTYMYEFR |
| | | (SEQ ID NO 751) |
| | | YISLEGFEQPVAVFLGVPFAKPPLGSLR |
| | | (SEQ ID NO 752) |
| | | YRPSFVSDK |
| | | (SEQ ID NO 753) |
| | | YRPSFVSDKRPQTVEGDHGDEIFFVFGAPLLK |
| | | (SEQ ID NO 754) |
| gi\|33563252 | Fibrinogen, alpha | ALTEMRQMR ox ox |
| | polypeptide | (SEQ ID NO 755) |
| | | AQQIQALQSNVR |
| | | (SEQ ID NO 756) |
| | | AVNREINLQDYEGHQK |
| | | (SEQ ID NO 757) |
| | | DSDWPFCSDDDWNHKCPSGCR cam cam cam |
| | | (SEQ ID NO 758) |
| | | EINLQDYEGHQK |
| | | (SEQ ID NO 759) |
| | | EINLQDYEGHQKQLQQVIAK |
| | | (SEQ ID NO 760) |
| | | GDFANANNFDNTYGQVSEDLR |
| | | (SEQ ID NO 761) |
| | | GDFANANNFDNTYGQVSEDLRR |
| | | (SEQ ID NO 762) |
| | | GDFATRGPGSK |
| | | (SEQ ID NO 763) |
| | | GDSRGDFATR |
| | | (SEQ ID NO 764) |
| | | GLIDEANQDFTNR |
| | | (SEQ ID NO 765) |
| | | GLIDEANQDFTNRINK |
| | | (SEQ ID NO 766) |
| | | HPDLSGFFDNHFGLISPNFKEFGSK |
| | | (SEQ ID NO 767) |
| | | LKNSLFDFQR |
| | | (SEQ ID NO 768) |
| | | MADEAGSEAHREGETR |
| | | (SEQ ID NO 769) |
| | | MKGLIDEANQDFTNR |
| | | (SEQ ID NO 770) |
| | | MSPVPDLVPGSFK ox |
| | | (SEQ ID NO 771) |
| | | MSPVPDLVPGSFKSQLQEAPPEWK ox |
| | | (SEQ ID NO 772) |
| | | NNKDSNSLTR |
| | | (SEQ ID NO 773) |
| | | QYLPALKMSPVPDLVPGSFK ox |
| | | (SEQ ID NO 774) |
| | | RLEVDIDIK |
| | | (SEQ ID NO 775) |
| | | SQLQEAPPEWK |
| | | (SEQ ID NO 776) |
| | | SQLQEAPPEWKALTEMR |
| | | (SEQ ID NO 777) |
| | | THSDSDILTNIEDPSSHVPEFSSSSK |
| | | (SEQ ID NO 778) |
| | | VIEKAQQIQALQSNVR |
| | | (SEQ ID NO 779) |
| | | VTSSGTSTTHR |
| | | (SEQ ID NO 780) |
| gi\|33859809 | Fibrinogen, B beta | AHYGGFTVQNEASK |
| | polypeptide | (SEQ ID NO 781) |
| | | AHYGGFTVQNEASKYQVSVNK |
| | | (SEQ ID NO 782) |
| | | EDGGGWWYNR |
| | | (SEQ ID NO 783) |
| | | EEPPSLRPAPPPISGGGYR |
| | | (SEQ ID NO 784) |
| | | ENENVINEYSSILEDQR |
| | | (SEQ ID NO 785) |
| | | GFGNIATNEDAK |
| | | (SEQ ID NO 786) |
| | | GFGNIATNEDAKK |
| | | (SEQ ID NO 787) |
| | | GGETSEMYLIQPDTSIKPYR |
| | | (SEQ ID NO 788) |
| | | GSWYSMRR ox |
| | | (SEQ ID NO 789) |
| | | HGTDDGVVWMNWK |
| | | (SEQ ID NO 790) |
| | | IQKLESDISAQMEYCR cam |
| | | (SEQ ID NO 791) |
| | | IRPFFPQQ |
| | | (SEQ ID NO 792) |
| | | KEEPPSLRPAPPPISGGGYR |
| | | (SEQ ID NO 793) |
| | | KGGETSEMYLIQPDTSIKPYR |
| | | (SEQ ID NO 794) |
| | | LYIDETVNDNIPLNLR |
| | | (SEQ ID NO 795) |
| | | LYIDETVNDNIPLNLRVLR |
| | | (SEQ ID NO 796) |
| | | MGPTELLIEMEDWKGDK |
| | | (SEQ ID NO 797) |
| | | MSMKIRPFFPQQ ox |
| | | (SEQ ID NO 798) |
| | | QAQVKENENVINEYSSILEDQR |
| | | (SEQ ID NO 799) |
| | | QCSKEDGGGWWYNR cam |
| | | (SEQ ID NO 800) |
| | | QDGSVDFGRK |
| | | (SEQ ID NO 801 |
| | | SILEDLRSK |
| | | (SEQ ID NO 802) |
| | | TENGGWTVIQNR |
| | | (SEQ ID NO 803) |
| | | TPCTVSCNIPVVSGK cam cam |
| | | (SEQ ID NO 804) |
| | | TPCTVSCNIPVVSGKECEEIIR cam cam cam |
| | | (SEQ ID NO 805) |
| | | VYCDMKTENGGWTVIQNR cam |
| | | (SEQ ID NO 806) |
| | | YCGLPGEYWLGNDK cam |
| | | (SEQ ID NO 807) |
| | | YCGLPGEYWLGNDKISQLTR cam |
| | | (SEQ ID NO 808) |
| | | YYWGGLYSWDMSK |
| | | (SEQ ID NO 809) |
| gi\|19527078 | Fibrinogen, gamma | AIQVYYNPDQPPKPGMIDSATQK |
| | polypeptide | (SEQ ID NO 810) |
| | | CHAGHLNGVYHQGGTYSK cam |
| | | (SEQ ID NO 811) |
| | | DNCCILDER cam cam |
| | | (SEQ ID NO 812) |
| | | EGFGHLSPTGTTEFWLGNEK |
| | | (SEQ ID NO 813) |
| | | ESGLYFIRPLK |
| | | (SEQ ID NO 814) |
| | | ESGLYFIRPLKAK |
| | | (SEQ ID NO 815) |
| | | FGSFCPTTCGIADFLSSYQTDVDNDLR cam cam |
| | | (SEQ ID NO 816) |
| | | GAKESGLYFIRPLK |
| | | (SEQ ID NO 817) |
| | | IHLISMQSTIPYALR |
| | | (SEQ ID NO 818) |
| | | IIPFNRLSIGEGQQHHMGGSK |
| | | (SEQ ID NO 819) |
| | | IQLKDWNGR |
| | | (SEQ ID NO 820) |
| | | LSIGEGQQHHMGGSK |
| | | (SEQ ID NO 821) |
| | | LSIGEGQQHHMGGSKQAGDV |
| | | (SEQ ID NO 822) |
| | | MVEEIVKYEALLLTHETSIR |
| | | (SEQ ID NO 823) |
| | | QQFLVYCEIDGSGNGWTVLQK cam |
| | | (SEQ ID NO 824) |
| | | QQFLVYCEIDGSGNGWTVLQKR cam |
| | | (SEQ ID NO 825) |
| | | RIDGSLDFK |
| | | (SEQ ID NO 826) |
| | | SSTTNGFDDGIIWATWK |
| | | (SEQ ID NO 827) |
| | | SSTTNGFDDGIIWATWKSR |
| | | (SEQ ID NO 828) |
| | | TSTADYAMFR |
| | | (SEQ ID NO 829) |
| | | WYSMKETTMK |
| | | (SEQ ID NO 830) |
| | | YLQEIYNSNNQK |
| | | (SEQ ID NO 831) |
| gi\|1181242 | Fibronectin | ATGVFTTLQPLR |
| | | (SEQ ID NO 832) |
| | | DGQERDAPIVNR |
| | | (SEQ ID NO 833) |
| | | DTLTSRPAQGVITTLENVSPPRR |
| | | (SEQ ID NO 834) |
| | | FLTTTPNSLLVSWQAPR |
| | | (SEQ ID NO 835) |
| | | FTNIGPDTMR |
| | | (SEQ ID NO 836) |
| | | GGQPKQYNVGPLASK |
| | | (SEQ ID NO 837) |
| | | GVTYNIIVEALQNQR |
| | | (SEQ ID NO 838) |
| | | GVTYNIIVEALQNQRR |
| | | (SEQ ID NO 839) |
| | | NLQPGSEYTATLVAVK |
| | | (SEQ ID NO 840) |
| | | HHAEHSVGRPR |
| | | (SEQ ID NO 841) |
| | | IAGYRLTAGLTR |
| | | (SEQ ID NO 842) |
| | | IHLYTLNDNAR |
| | | (SEQ ID NO 843) |
| | | ITGYIIKYEKPGSPPR |
| | | (SEQ ID NO 844) |
| | | ITYGETGGNSPVQEFTVPGSK |
| | | (SEQ ID NO 845) |
| | | LGVRPSQGGEAPR |
| | | (SEQ ID NO 846) |
| | | LTCQCLGFGSGHFR cam cam |
| | | (SEQ ID NO 847) |
| | | NTFAEITGLSPGVTYLFK |
| | | (SEQ ID NO 848) |
| | | QYNVGPLASKYPLR |
| | | (SEQ ID NO 849) |
| | | SSPVIIDASTAIDAPSNLR |
| | | (SEQ ID NO 850) |
| | | STTPDITGYR |
| | | (SEQ ID NO 851) |
| | | TKTETITGFQVDAIPANGQTPVQR |
| | | (SEQ ID NO 852) |
| | | VTDATETTITISWR |
| | | (SEQ ID NO 853) |
| | | VTWAPPPSIELTNLLVR |
| | | (SEQ ID NO 854) |
| | | WLPSTSPVTGYR |
| | | (SEQ ID NO 855) |
| | | TVLVTWTPPR |
| | | (SEQ ID NO 856) |
| gi\|46849812 | Fibronectin 1 | APITGYIIR |
| | | (SEQ ID NO 857) |
| | | ATGVFTTLQPLR |
| | | (SEQ ID NO 858) |
| | | CDPIDQCQDSETR cam cam |
| | | (SEQ ID NO 859) |
| | | CHEGGQSYK cam |
| | | (SEQ ID NO 860) |
| | | DGQERDAPIVNR |
| | | (SEQ ID NO 861) |
| | | DQCIVDDITYNVNDTFHK cam |
| | | (SEQ ID NO 862) |
| | | EYLGAICSCTCFGGQR cam cam cam |
| | | (SEQ ID NO 863) |
| | | FLTTTPNSLLVSWQAPR |
| | | (SEQ ID NO 864) |
| | | FTNIGPDTMR |
| | | (SEQ ID NO 865) |
| | | GDSPASSKPVSINYK |
| | | (SEQ ID NO 866) |
| | | GEWACIPYSQLR cam |
| | | (SEQ ID NO 867) |
| | | GGNSNGALCHFPFLYNNR cam |
| | | (SEQ ID NO 868) |
| | | GGQPKQYNVGPLASK |
| | | (SEQ ID NO 869) |
| | | GLTPGVIYEGQLISIQQYGHR |
| | | (SEQ ID NO 870) |
| | | GNLLQCVCTGNGR cam cam |
| | | (SEQ ID NO 871) |
| | | GRISCTIANR cam |
| | | (SEQ ID NO 872) |
| | | GVTYNIIVEALQNQR |
| | | (SEQ ID NO 873) |
| | | GVTYNIIVEALQNQRR |
| | | (SEQ ID NO 874) |
| | | HALQSASAGSGSFTDVR |
| | | (SEQ ID NO 875) |
| | | HHAEHSVGRPR |
| | | (SEQ ID NO 876) |
| | | HYQINQQWER |
| | | (SEQ ID NO 877) |
| | | IAGYRLTAGLTR |
| | | (SEQ ID NO 878) |
| | | IGDQWDKQHDLGHMMR ox |
| | | (SEQ ID NO 879) |
| | | IGDTWSK |
| | | (SEQ ID NO 880) |
| | | IGEKWDR |
| | | (SEQ ID NO 881) |
| | | IHLYTLNDNAR |
| | | (SEQ ID NO 882) |
| | | ISCTIANR cam |
| | | (SEQ ID NO 883) |
| | | ITGYIIKYEKPGSPPR |
| | | (SEQ ID NO 884) |
| | | ITYGETGGNSPVQEFTVPGSK |
| | | (SEQ ID NO 885) |
| | | LGVRPSQGGEAPR |
| | | (SEQ ID NO 886) |
| | | LTCQCLGFGSGHFR cam cam |
| | | (SEQ ID NO 887) |
| | | MSCTCLGNGKGEFK cam cam |
| | | (SEQ ID NO 888) |
| | | NLQPGSEYTVTLVAVKGNQQSPK |
| | | (SEQ ID NO 889) |
| | | NRCNDQDTR cam |
| | | (SEQ ID NO 890) |
| | | NTFAEITGLSPGVTYLFK |
| | | (SEQ ID NO 891) |
| | | QDRVPPSR |
| | | (SEQ ID NO 892) |
| | | QYNVGPLASKYPLR |
| | | (SEQ ID NO 893) |
| | | RPGAAEPSPDGTTGHTYNQYTQR |
| | | (SEQ ID NO 894) |
| | | SDNVPPPTDLQFVELTDVK |
| | | (SEQ ID NO 895) |
| | | SSPVIIDASTAIDAPSNLR |
| | | (SEQ ID NO 896) |
| | | STATINNIKPGADYTITLYAVTGR |
| | | (SEQ ID NO 897) |
| | | STTPDITGYR |
| | | (SEQ ID NO 898) |
| | | TEIDKPSQMQVTDVQDNSISVR ox |
| | | TFYQIGDSWEK |
| | | (SEQ ID NO 899) |
| | | TFYSCTTEGR cam |
| | | (SEQ ID NO 900) |
| | | TKTETITGFQVDAIPANGQTPVQR |
| | | (SEQ ID NO 901) |
| | | VEVLPVSLPGEHGQR |
| | | (SEQ ID NO 902) |
| | | VFAVHQGR |
| | | (SEQ ID NO 903) |
| | | VTDATETTITISWR |
| | | (SEQ ID NO 904) |
| | | VTWAPPPSIELTNLLVR |
| | | (SEQ ID NO 905) |
| | | WCHDNGVNYK cam |
| | | (SEQ ID NO 906) |
| | | WKCDPIDQCQDSETR cam cam |
| | | (SEQ ID NO 907) |
| | | WKEATIPGHLNSYTIK |
| | | (SEQ ID NO 908) |
| | | WLPSTSPVTGYR |
| | | (SEQ ID NO 909) |
| | | WSRPQAPITGYR |
| | | (SEQ ID NO 910) |
| | | YEKPGSPPR |
| | | (SEQ ID NO 911) |
| | | YIVNVYQISEEGK |
| | | (SEQ ID NO 912) |
| | | YSFCTDHAVLVQTR cam |
| | | (SEQ ID NO 913) |
| | | YTGNTYK |
| | | (SEQ ID NO 914) |
| gi\|28916693 | Gelsolin | AGKEPGLQIWR |
| | | (SEQ ID NO 915) |
| | | AVQHREVQGFESSTFSGYFK |
| | | (SEQ ID NO 916) |
| | | DGGQTAPASIR |
| | | (SEQ ID NO 917) |
| | | DPDQTDGPGLGYLSSHIANVER |
| | | (SEQ ID NO 918) |
| | | EPAHLMSLFGGKPMIIYK |
| | | (SEQ ID NO 919) |
| | | EVQGFESSTFSGYFK |
| | | (SEQ ID NO 920) |
| | | FDLVPVPPNLYGDFFTGDAYVILK |
| | | (SEQ ID NO 921) |
| | | GGVASGFKHVVPNEVVVQR |
| | | (SEQ ID NO 922) |
| | | HVVPNEVVVQR |
| | | (SEQ ID NO 923) |
| | | IEGSNKVPVDPATYGQFYGGDSYIILYNYR |
| | | (SEQ ID NO 924) |
| | | MDAHPPRLFACSNR ox cam |
| | | (SEQ ID NO 925) |
| | | NWRDPDQTDGPGLGYLSSHIANVER |
| | | (SEQ ID NO 926) |
| | | QTQVSVLPEGGETPLFKQFFK |
| | | (SEQ ID NO 927) |
| | | SEDCFILDHGR cam |
| | | (SEQ ID NO 928) |
| | | SEDCFILDHGRDGK cam |
| | | (SEQ ID NO 929) |
| | | SGALNSNDAFVLK |
| | | (SEQ ID NO 930) |
| | | SQHVQVEEGSEPDAFWEALGGK |
| | | (SEQ ID NO 931) |
| | | TASDFISKMQYPR ox |
| | | (SEQ ID NO 932) |
| | | TPSAAYLWVGAGASEAEK |
| | | (SEQ ID NO 933) |
| | | TPSAAYLWVGAGASEAEKTGAQELLK |
| | | (SEQ ID NO 934) |
| | | VEKFDLVPVPPNLYGDFFTGDAYVILK |
| | | (SEQ ID NO 935) |
| | | VPFDAATLHTSTAMAAQHGMDDDGTGQK |
| | | (SEQ ID NO 936) |
| | | VPVDPATYGQFYGGDSYIILYNYR |
| | | (SEQ ID NO 937) |
| | | VSEARPSTMVVEHPEFLK |
| | | (SEQ ID NO 938) |
| | | VSNGAGSMSVSLVADENPFAQGALR |
| | | (SEQ ID NO 939) |
| | | VVQGKEPAHLMSLFGGKPMIIYK ox |
| | | (SEQ ID NO 940) |
| | | YIETDPANR |
| | | (SEQ ID NO 941) |
| | | YIETDPANRDR |
| | | (SEQ ID NO 942) |
| gi\|52843238 | Glutathione | FLVGPDGIPVMR ox |
| | peroxidase 3 | (SEQ ID NO 943) |
| | | LFWEPMKIHDIR |
| | | (SEQ ID NO 944) |
| | | MDILSYMRR ox |
| | | (SEQ ID NO 945) |
| | | NSCPPTAELLGSPGR cam |
| | | (SEQ ID NO 946) |
| | | QEPGENSEILPSLK |
| | | (SEQ ID NO 947) |
| | | TTVSNVKMDILSYMR ox |
| | | (SEQ ID NO 948) |
| | | WNFEKFLVGPDGIPVMR ox |
| | | (SEQ ID NO 949) |
| | | YVRPGGGFVPNFQLFEK |
| | | (SEQ ID NO 950) |
| | | YVRPGGGFVPNFQLFEKGDVNGEK |
| | | (SEQ ID NO 951) |
| gi\|17512357 | Gpld1 protein | AQYVLTSPEASSR |
| | | (SEQ ID NO 952) |
| | | GIVATFYSHPR |
| | | (SEQ ID NO 953) |
| | | GKYHDVSER |
| | | (SEQ ID NO 954) |
| | | GRNQVVVAAGR |
| | | (SEQ ID NO 955) |
| | | IYDNLYGRK |
| | | (SEQ ID NO 956) |
| | | LGTSLSSGYVR |
| | | (SEQ ID NO 957) |
| | | LSGALHVYSFSSD |
| | | (SEQ ID NO 958) |
| | | NDFHRNLTMFISR ox |
| | | (SEQ ID NO 959) |
| | | NLRLMLAGSSQK ox |
| | | (SEQ ID NO 960) |
| | | NLTMFISRDIR |
| | | (SEQ ID NO 961) |
| | | SLGKVYGYFLPNR |
| | | (SEQ ID NO 962) |
| | | SSWGARLSGALHVYSFSSD |
| | | (SEQ ID NO 963) |
| | | TQPALLSTFSGDRR |
| | | (SEQ ID NO 964) |
| | | VYGYFLPNR |
| | | (SEQ ID NO 965) |
| | | WYVPVRDLLR |
| | | (SEQ ID NO 966) |
| | | YHDVSERTHWTPFLNASIHYIR |
| | | (SEQ ID NO 967) |
| gi\|1694789 | GRS protein | LAQDYLQCVLQIPQPGSGPSK cam |
| | | (SEQ ID NO 968) |
| | | QNGGWENGFVKK |
| | | (SEQ ID NO 969) |
| | | SGWMTFLEVTGKICEMLSLLK cam |
| | | (SEQ ID NO 970) |
| | | TLFTQVMEK |
| | | (SEQ ID NO 971) |
| | | VLQNVAFSVQKEVEK |
| | | (SEQ ID NO 972) |
| gi\|37719755 | GUGU alpha | AMFHINKPR |
| | | (SEQ ID NO 973) |
| | | AMFHINKPRR |
| | | (SEQ ID NO 974) |
| | | AMNQWVSGPAYYVEYLIK |
| | | (SEQ ID NO 975) |
| | | DGYMLSLNR ox |
| | | (SEQ ID NO 976) |
| | | DQKDGYMLSLNR |
| | | (SEQ ID NO 977) |
| | | EHYQEDMGSLFYLTLDVLETDCHVLSR ox cam |
| | | (SEQ ID NO 978) |
| | | GSIQHLPELDDEKPEESK |
| | | (SEQ ID NO 979) |
| | | HVPLIQPVEK |
| | | (SEQ ID NO 980) |
| | | ISAFDRFGR |
| | | (SEQ ID NO 981) |
| | | KTHTTCPDCPSPIDLSNPSALEAATESLAK cam cam |
| | | (SEQ ID NO 982) |
| | | LVVLPFPGKEQR |
| | | (SEQ ID NO 983) |
| | | MFYESVYGQCK cam |
| | | (SEQ ID NO 984) |
| | | MISAFDRFGR |
| | | (SEQ ID NO 985) |
| | | SAECPGPEKENNPLVLPP cam |
| | | (SEQ ID NO 986) |
| | | SQASCSLQHSDSEPVGICQGSTVQSSLR cam cam |
| | | (SEQ ID NO 987) |
| | | THTTCPDCPSPIDLSNPSALEAATESLAK cam cam |
| | | (SEQ ID NO 988) |
| gi\|23956086 | Hemopexin | DYFVSCPGR cam |
| | | (SEQ ID NO 989) |
| | | ELGSPPGISLETIDAAFSCPGSSR cam |
| | | (SEQ ID NO 990) |
| | | FNPVTGEVPPR |
| | | (SEQ ID NO 991) |
| | | FNPVTGEVPPRYPLDAR |
| | | (SEQ ID NO 992) |
| | | GATYAFTGSHYWR |
| | | (SEQ ID NO 993) |
| | | GECQSEGVLFFQGNR cam |
| | | (SEQ ID NO 994) |
| | | GECQSEGVLFFQGNRK cam |
| | | (SEQ ID NO 995) |
| | | GGNNLVSGYPK |
| | | (SEQ ID NO 996) |
| | | GPDSVFLIK |
| | | (SEQ ID NO 997) |
| | | LFQEEFPGIPYPPDAAVECHR cam |
| | | (SEQ ID NO 998) |
| | | LWWLDLK |
| | | (SEQ ID NO 999) |
| | | LYVSSGRR |
| | | (SEQ ID NO 1000) |
| | | RLWWLDLK |
| | | (SEQ ID NO 1001) |
| | | SGAQATWTEVSWPHEK |
| | | (SEQ ID NO 1002) |
| | | SGAQATWTEVSWPHEKVDGALCLDK cam |
| | | (SEQ ID NO 1003) |
| | | SLGPNTCSSNGSSLYFIHGPNLYCYSSIDKLNAAK |
| | | cam cam |
| | | (SEQ ID NO 1004) |
| | | SLPQPQKVNSILGCSQ cam |
| | | (SEQ ID NO 1005) |
| | | VWVYPPEKK |
| | | (SEQ ID NO 1006) |
| | | WFWDFATR |
| | | (SEQ ID NO 1007) |
| | | WKNPITSVDAAFR |
| | | (SEQ ID NO 1008) |
| | | WLERYYCFQGNK cam |
| | | (SEQ ID NO 1009) |
| | | YYCFQGNK cam |
| | | (SEQ ID NO 1010) |
| | | YYCFQGNKFLR cam |
| | | (SEQ ID NO 1011) |
| gi\|50082914 | High molecular | ATSQVVAGTKYVIEFIAR |
| | weight kininogen I | (SEQ ID NO 1012) |
| | isoform DeltaD5 | AYFPCIGCVHAISTDSPDLEPVLK cam cam |
| | | (SEQ ID NO 1013) |
| | | CQALDMTEMAR ox cam |
| | | (SEQ ID NO 1014) |
| | | ENEFFIVTQTCK cam |
| | | (SEQ ID NO 1015) |
| | | FPSLHGDCVALPNGDDGECR cam cam |
| | | (SEQ ID NO 1016) |
| | | LISDFPEATSPK |
| | | (SEQ ID NO 1017) |
| | | RENEFFIVTQTCK cam |
| | | (SEQ ID NO 1018) |
| | | RPPGFSPFR |
| | | (SEQ ID NO 1019) |
| | | SGNQYMLHR |
| | | (SEQ ID NO 1020) |
| | | TDGSPTFYSFK |
| | | (SEQ ID NO 1021) |
| | | VIEGTKTDGSPTFYSFK |
| | | (SEQ ID NO 1022) |
| gi\|40715898 | HMW-kininogen-II | AKMDGSATFYSFNYQIK ox |
| | variant | (SEQ ID NO 1023) |
| | | ATSQVVAGTNYVIEFIAR |
| | | (SEQ ID NO 1024) |
| | | CQALDKTIPIR cam |
| | | (SEQ ID NO 1025) |
| | | EGNCSAQRGLAWQDCDFK cam cam |
| | | (SEQ ID NO 1026) |
| | | ENKFFIVTQTCK cam |
| | | (SEQ ID NO 1027) |
| | | FFIVTQTCK cam |
| | | (SEQ ID NO 1028) |
| | | GDCVALPNGDGGECR cam cam |
| | | (SEQ ID NO 1029) |
| | | KATSQVVAGTNYVIEFIAR |
| | | (SEQ ID NO 1030) |
| | | MDGSATFYSFNYQIK ox |
| | | (SEQ ID NO 1031) |
| | | QLPRNVLGAHGSQLAGR |
| | | (SEQ ID NO 1032) |
| | | RPPGFSPFR |
| | | (SEQ ID NO 1033) |
| | | RRPPGFSPFR |
| | | (SEQ ID NO 1034) |
| gi\|31615671 | Ig (A Chain A, Crystal | ASQSISNNLHWYQQK |
| | Structure Of Fab | (SEQ ID NO 1035) |
| | Fragment Of | DIVLTQSPATLSVTPGDSVSLSCR cam |
| | Antibody Hyhel-26 | (SEQ ID NO 1036) |
| | Complexed With | DSTYSMSSTLTLTKDEYER |
| | Lysozyme) | (SEQ ID NO 1037) |
| | | HNSYTCEATHK cam |
| | | (SEQ ID NO 1038) |
| | | QNGVLNSWTDQDSK |
| | | (SEQ ID NO 1039) |
| | | YASQSISGIPSR |
| | | (SEQ ID NO 1040) |
| gi\|4930001 | Ig (A Chain A, | IAVAWYQQKPGQSPK |
| | Idiotope-Anti-Idiotope | (SEQ ID NO 1041) |
| | Fab-Fab Complex) | DSTYSMSSTLTLTKDEYER ox |
| | | (SEQ ID NO 1042) |
| | | FMSTSVGDRVSITCK cam |
| | | (SEQ ID NO 1043) |
| | | HNSYTCEATHK cam |
| | | (SEQ ID NO 1044) |
| | | LLIYWASTR |
| | | (SEQ ID NO 1045) |
| | | QNGVLDSWTDQDSK |
| | | (SEQ ID NO 1046) |
| | | VSITCKASQDVR cam |
| | | (SEQ ID NO 1047) |
| gi\|11514687 | Ig (A Chain A, Lyme | ASQDINKYIAWYQHKPGK |
| | Disease Antigen | (SEQ ID NO 1048) |
| | Ospa In Complex | DSTYSMSSTLTLTKDEYER ox |
| | With Neutralizing | (SEQ ID NO 1049) |
| | Antibody Fab La-2) | HNSYTCEATHK cam |
| | | (SEQ ID NO 1050) |
| | | HNSYTCEATHKTSTSPIVK cam |
| | | (SEQ ID NO 1051) |
| | | QNGVLNSWTDQDSK |
| | | (SEQ ID NO 1052) |
| | | RADAAPTVSIFPPSSEQLTSGGASVVCFLNNFYPK |
| | | cam |
| | | (SEQ ID NO 1053) |
| | | YIAWYQHKPGK |
| | | (SEQ ID NO 1054) |
| gi\|42543442 | Ig (A Chain A, S25-2- | DIVMSQSPSSLAVSAGEK ox |
| | Kdo Monosaccharide | (SEQ ID NO 1055) |
| | Complex) | DSTYSMSSTLTLTKDEYER ox |
| | | (SEQ ID NO 1056) |
| | | HNSYTCEATHK cam |
| | | (SEQ ID NO 1057) |
| | | LLIYWASTR |
| | | (SEQ ID NO 1058) |
| | | NYLAWYQQKPGQSPK |
| | | (SEQ ID NO 1059) |
| | | QNGVLNSWTDQDSK |
| | | (SEQ ID NO 1060) |
| | | TSTSPIVKSFNR |
| | | (SEQ ID NO 1061) |
| gi\|7766934 | Ig (A Chain A, | ADAAPTVSIFPPSSEQLTSGGASVVCFLNNFYPK |
| | Structure Of An | cam |
| | Activity Suppressing | (SEQ ID NO 1062) |
| | Fab Fragment To | DIVLTQSPASLAVSLGQR |
| | Cytochrome P450 | (SEQ ID NO 1063) |
| | Aromatase) | DIVLTQSPASLAVSLGQRATISCR cam |
| | | (SEQ ID NO 1064) |
| | | HNSYTCEATHK cam |
| | | (SEQ ID NO 1065) |
| | | HNSYTCEATHKTSTSPIVK cam |
| | | (SEQ ID NO 1066) |
| | | LLIYLVSNLESGVPAR |
| | | (SEQ ID NO 1067) |
| gi\|27373551 | Ig (antibody variable | DFQPGYNTGLK |
| | domain) | (SEQ ID NO 1068) |
| | | FTISKTSTTVDLK |
| | | (SEQ ID NO 1069) |
| | | GLEYIGFINPRGTPYYASWAK |
| | | (SEQ ID NO 1070) |
| | | GTPYYASWAK |
| | | (SEQ ID NO 1071) |
| | | IWGPGTLVTVSS |
| | | (SEQ ID NO 1072) |
| gi\|1870378 | Ig (Anti-DNA | ASQSISDYLHWYQQK |
| | immunoglobulin light | (SEQ ID NO 1073) |
| | chain IgG) | ASQSISDYLHWYQQKSHESPR |
| | | (SEQ ID NO 1074) |
| | | DIVMTQSPATLSVTPGDR ox |
| | | (SEQ ID NO 1075) |
| | | DIVMTQSPATLSVTPGDRVSLSCR ox cam |
| | | (SEQ ID NO 1076) |
| | | LLIKYASQSISGIPSR |
| | | (SEQ ID NO 1077) |
| | | YASQSISGIPSR |
| | | (SEQ ID NO 1078) |
| gi\|l2002896 | Ig (anti-human | ADAAPTVSIFPPSSEQLTSGGASVVCFLNNFYPK |
| | apolipoprotein A | cam |
| | monoclonal antibody | (SEQ ID NO 1079) |
| | mAb(a)23L kappa | HNSYTCEATHK cam |
| | light chain) | (SEQ ID NO 1080) |
| | | HNSYTCEATHKTSTSPIVK cam |
| | | (SEQ ID NO 1081) |
| | | LLIHYTSTLQPGIPSR |
| | | (SEQ ID NO 1082) |
| | | RADAAPTVSIFPPSSEQLTSGGASVVCFLNNFYPK |
| | | cam |
| | | (SEQ ID NO 1083) |
| | | TFGGGTKLEIK |
| | | (SEQ ID NO 1084) |
| | | YIAWYQHKPGKGPR |
| | | (SEQ ID NO 1085) |
| gi\|349893 | Ig (C Chain C, Fab | DSTYSMSSTLTLTKDEYER |
| | (Igg2a, Kappa) | (SEQ ID NO 1086) |
| | Fragment (26-10) | DVVMTQTPLSLPVSLGDQASISCR cam |
| | Complex With | (SEQ ID NO 1087) |
| | Digoxin) | FSGSGSGTDFTLK |
| | | (SEQ ID NO 1088) |
| | | FSGVPDRFSGSGSGTDFTLK |
| | | (SEQ ID NO 1089) |
| | | HNSYTCEATHK cam |
| | | (SEQ ID NO 1090) |
| | | HNSYTCEATHKTSTSPIVK cam |
| | | (SEQ ID NO 1091) |
| | | QNGVLNSWTDQDSK |
| | | (SEQ ID NO 1092) |
| | | TSTSPIVKSFNR |
| | | (SEQ ID NO 1093) |
| | | VSNRFSGVPDR |
| | | (SEQ ID NO 1094) |
| gi\|47059057 | Ig (gamma-2b- | APQVYILPPPAEQLSR |
| | immunoglobulin) | (SEQ ID NO 1095) |
| | | CPAPNLEGGPSVFIFPPNIKDVLMISLTPK cam ox |
| | | (SEQ ID NO 1096) |
| | | LEPSGPISTINPCPPCKECHK cam cam cam |
| | | (SEQ ID NO 1097) |
| | | SEDTAMYYCAR cam |
| | | (SEQ ID NO 1098) |
| | | TTPPSVYPLAPGCGDTTGSSVTLGCLVK cam cam |
| | | (SEQ ID NO 1099) |
| | | VNNKDLPSPIER |
| | | (SEQ ID NO 1100) |
| | | VVSALPIQHQDWMSGKEFK ox |
| | | (SEQ ID NO 1101) |
| gi\|1806128 | Ig (immunoglobulin | ALPSPIEKTISKPR |
| | constant heavy chain) | (SEQ ID NO 1102) |
| | | APQVYVLPPPAEEMTKK |
| | | (SEQ ID NO 1103) |
| | | ECPPCAAPDLLGGPSVFIFPPK cam cam |
| | | (SEQ ID NO 1104) |
| | | GPVRAPQVYVLPPPAEEMTK |
| | | (SEQ ID NO 1105) |
| | | GSLFACSVVHEGLHNHLTTK cam |
| | | (SEQ ID NO 1106) |
| | | NTATVLDSDGSYFMYSK |
| | | (SEQ ID NO 1107) |
| | | TEQNYKNTATVLDSDGSYFMYSK ox |
| | | (SEQ ID NO 1108) |
| | | TTAPSVYPLAPVCGGTTGSSVTLGCLVK cam cam |
| | | (SEQ ID NO 1109) |
| | | VPITQNPCPPLK cam |
| | | (SEQ ID NO 1110) |
| | | VPITQNPCPPLKECPPCAAPDLLGGPSVFIFPPK cam |
| | | cam cam |
| | | (SEQ ID NO 1111) |
| | | VVSALPIQHQDWMSGK |
| | | (SEQ ID NO 1112) |
| | | VVSALPIQHQDWMSGKEFK |
| | | (SEQ ID NO 1113) |
| gi\|10121892 | Ig (immunoglobulin | ALIYSASYR |
| | IgM MP-18-3-117 | (SEQ ID NO 1114) |
| | kappa light chain) | ASQNVGTNVAWYQQKPGQSPK |
| | | (SEQ ID NO 1115) |
| | | FMSTSVGDRVSVTCK cam |
| | | (SEQ ID NO 1116) |
| | | FMSTSVGDRVSVTCK ox cam |
| | | (SEQ ID NO 1117) |
| | | HNSYTCEATHK cam |
| | | (SEQ ID NO 1118) |
| | | HNSYTCEATHKTSTSPIVK cam |
| | | (SEQ ID NO 1119) |
| | | RADAAPTVSIFPPSSEQLTSGGASVVCFLNNFYPK |
| | | cam |
| | | (SEQ ID NO 1120) |
| gi\|196723 | Ig (immunoglobulin | DVQITQSPSYLAASPGETITINCR cam |
| | kappa-chain VK-1) | (SEQ ID NO 1121) |
| | | DVQITQSPSYLAASPGETITINCRASK cam |
| | | (SEQ ID NO 1122) |
| | | LLIYSGSTLQSGIPSR |
| | | (SEQ ID NO 1123) |
| | | SISKYLAWYQEKPGK |
| | | (SEQ ID NO 1124) |
| | | TNKLLIYSGSTLQSGIPSR |
| | | (SEQ ID NO 1125) |
| | | YLAWYQEKPGK |
| | | (SEQ ID NO 1126) |
| | | YLAWYQEKPGKTNK |
| | | (SEQ ID NO 1127) |
| gi\|18033701 | Ig (immunoglobulin | ANGTPITQGVDTSNPTK |
| | light chain constant | (SEQ ID NO 1128) |
| | region) | EGNKFMASSFLHLTSDQWR |
| | | (SEQ ID NO 1129) |
| | | FMASSFLHLTSDQWR |
| | | (SEQ ID NO 1130) |
| | | SHNSFTCQVTHEGDTVEK cam |
| | | (SEQ ID NO 1131) |
| | | STPTLTVFPPSSEELKENK |
| | | (SEQ ID NO 1132) |
| gi\|13399686 | Ig (L Chain L, 64m-2 | ADAAPTVSIFPPSSEQLTSGGASVVCFLNNFYPK |
| | Antibody Fab | cam |
| | Complexed With | (SEQ ID NO 1133) |
| | D(5ht)(6-4)t) | DSTYSMSSTLTLTKDEYER |
| | | (SEQ ID NO 1134) |
| | | DVLMTQTPLSLPVSLGDQASISCR cam |
| | | (SEQ ID NO 1135) |
| | | FSGSGSGTDFTLK |
| | | (SEQ ID NO 1136) |
| | | FSGVPDRFSGSGSGTDFTLK |
| | | (SEQ ID NO 1137) |
| | | HNSYTCEATHK cam |
| | | (SEQ ID NO 1138) |
| | | QNGVLNSWTDQDSK |
| | | (SEQ ID NO 1139) |
| | | RADAAPTVSIFPPSSEQLTSGGASVVCFLNNFYPK |
| | | cam |
| | | (SEQ ID NO 1140) |
| | | SSQSIVHSNGNTYLEWYLQKPGQSPK |
| | | (SEQ ID NO 1141) |
| | | VSNRFSGVPDR |
| | | (SEQ ID NO 1142) |
| gi\|3212470 | Ig (L Chain L, Anti | ADAAPTVSIFPPSSEQLTSGGASVVCFLNNFYPK |
| | Taq Fab Tp7) | cam |
| | | (SEQ ID NO 1143) |
| | | DEYERHNSYTCEATHK cam |
| | | (SEQ ID NO 1144) |
| | | DIQMTQSPAIMSASPGEK ox ox |
| | | (SEQ ID NO 1145) |
| | | DSTYSMSSTLTLTKDEYER |
| | | (SEQ ID NO 1146) |
| | | FSGSGSGTSYSLTISR |
| | | (SEQ ID NO 1147) |
| | | HNSYTCEATHK cam |
| | | (SEQ ID NO 1148) |
| | | HNSYTCEATHKTSTSPIVK cam |
| | | (SEQ ID NO 1149) |
| | | LLIYDSTNLASGVPVR |
| | | (SEQ ID NO 1150) |
| | | RADAAPTVSIFPPSSEQLTSGGASVVCFLNNFYPK |
| | | cam |
| | | (SEQ ID NO 1151) |
| gi\|5542523 | Ig (L Chain L, | ADAAPTVSIFPPSSEQLTSGGASVVCFLNNFYPK |
| | Bactericidal Antibody | cam |
| | Against Neisseria | (SEQ ID NO 1152) |
| | Meningitidis) | DIVMTQTPLSLPVSLGDKASISCR cam |
| | | (SEQ ID NO 1153) |
| | | DIVMTQTPLSLPVSLGDKASISCR ox cam |
| | | (SEQ ID NO 1154) |
| | | DSTYSMSSTLTLTKDEYER ox |
| | | (SEQ ID NO 1155) |
| | | FSGSGSGTDFTLK |
| | | (SEQ ID NO 1156) |
| | | FSGVPDRFSGSGSGTDFTLK |
| | | (SEQ ID NO 1157) |
| | | HNSYTCEATHK cam |
| | | (SEQ ID NO 1158) |
| | | HNSYTCEATHKTSTSPIVK cam |
| | | (SEQ ID NO 1159) |
| | | QNGVLNSWTDQDSK |
| | | (SEQ ID NO 1160) |
| | | TFGGGTKLEIK |
| | | (SEQ ID NO 1161) |
| | | VSNRFSGVPDR |
| | | (SEQ ID NO 1162) |
| gi\|16975338 | Ig (L Chain L, Crystal | ADAAPTVSIFPPSSEQLTSGGASVVCFLNNFYPK |
| | Structure Of | cam |
| | Immunoglobulin Fab | (SEQ ID NO 1163) |
| | Fragment Complexed | ASGNIHNYLAWYQQK |
| | With 17-Beta- | (SEQ ID NO 1164) |
| | Estradiol) | DEYERHNSYTCEATHK cam |
| | | (SEQ ID NO 1165) |
| | | DIQMTQSPASLSASVGETVTITCR ox cam |
| | | (SEQ ID NO 1166) |
| | | DSTYSMSSTLTLTKDEYER |
| | | (SEQ ID NO 1167) |
| | | DSTYSMSSTLTLTKDEYER ox |
| | | (SEQ ID NO 1168) |
| | | FSGSGSGTQYSLK |
| | | (SEQ ID NO 1169) |
| | | HNSYTCEATHK cam |
| | | (SEQ ID NO 1170) |
| | | HNSYTCEATHKTSTSPIVK cam |
| | | (SEQ ID NO 1171) |
| | | QNGVLNSWTDQDSK |
| | | (SEQ ID NO 1172) |
| | | SPQLLVYNAK |
| | | (SEQ ID NO 1173) |
| gi\|18655521 | Ig (L Chain L, Crystal | ADAAPTVSIFPPSSEQLTSGGASVVCFLNNFYPK |
| | Structure Of The Fab | cam |
| | Fragment Of The | (SEQ ID NO 1174) |
| | Mouse Anti- Human | ASQSVDYDGDSYMNWYQQKPGQPPK |
| | Fas Antibody Hfe7a) | (SEQ ID NO 1175) |
| | | ASQSVDYDGDSYMNWYQQKPGQPPK ox |
| | | (SEQ ID NO 1176) |
| | | DIVLTQSPASLAVSLGQR |
| | | (SEQ ID NO 1177) |
| | | DSTYSMSSTLTLTKDEYER |
| | | (SEQ ID NO 1178) |
| | | DSTYSMSSTLTLTKDEYER ox |
| | | (SEQ ID NO 1179) |
| | | HNSYTCEATHK cam |
| | | (SEQ ID NO 1180) |
| | | LLIYAASNLESGIPAR |
| | | (SEQ ID NO 1181) |
| gi\|1942810 | Ig (L Chain L, Fab | ADAAPTVSIFPPSSEQLTSGGASVVCFLNNFYPK |
| | Fragment Of A | cam |
| | Neutralizing Antibody | (SEQ ID NO 1182) |
| | Directed Against An | DVVMTQTPLTLSVTIGQPASISCK cam |
| | Epitope Of Gp41 | (SEQ ID NO 1183) |
| | From Hiv-1) | DVVMTQTPLTLSVTIGQPASISCK ox cam |
| | | (SEQ ID NO 1184) |
| | | FTGSGSGTDFTLK |
| | | (SEQ ID NO 1185) |
| | | HNSYTCEATHK cam |
| | | (SEQ ID NO 1186) |
| | | HNSYTCEATHKTSTSPIVK cam |
| | | (SEQ ID NO 1187) |
| | | LDSGVPDRFTGSGSGTDFTLK |
| | | (SEQ ID NO 1188) |
| | | LIYLVSKLDSGVPDR |
| | | (SEQ ID NO 1189) |
| | | QNGVLNSWTDQDSK |
| | | (SEQ ID NO 1190) |
| | | SSQSLLDSDGKTYLNWLLQRPGQSPK |
| | | (SEQ ID NO 1191) |
| | | TFGGGTKLEIK |
| | | (SEQ ID NO 1192) |
| | | VEAEDLGVYYCWQGTHFPR cam |
| | | (SEQ ID NO 1193) |
| gi\|7546516 | Ig (L Chain L, Fab | ADAAPTVSIFPPSSEQLTSGGASVVCFLNNFYPK |
| | Fragment Of | cam |
| | Neutralising | (SEQ ID NO 1194) |
| | Monoclonal Antibody | DIVLTQSPASLAVSLGQR |
| | 4c4 Complexed With | (SEQ ID NO 1195) |
| | G-H Loop From | HNSYTCEATHK cam |
| | Fmdv) | (SEQ ID NO 1196) |
| | | LLIYRASNLESGIPAR |
| | | (SEQ ID NO 1197) |
| | | RADAAPTVSIFPPSSEQLTSGGASVVCFLNNFYPK |
| | | cam |
| | | (SEQ ID NO 1198) |
| gi\|17943084 | Ig (L Chain L, | DSTYSMSSTLTLTKDEYER |
| | Structural Basis For | (SEQ ID NO 1199) |
| | Disfavored | DSTYSMSSTLTLTKDEYER ox |
| | Elimination Reaction | (SEQ ID NO 1200) |
| | In Catalytic Antibody | EVVMTQSPLSLPVSLGDQASISCR cam |
| | 1d4) | (SEQ ID NO 1201) |
| | | EVVMTQSPLSLPVSLGDQASISCR ox cam |
| | | (SEQ ID NO 1202) |
| | | FSGSGSGTDFTLK |
| | | (SEQ ID NO 1203) |
| | | FSGVPDRFSGSGSGTDFTLK |
| | | (SEQ ID NO 1204) |
| | | HNSYTCEATHK cam |
| | | (SEQ ID NO 1205) |
| | | HNSYTCEATHKTSTSPIVK cam |
| | | (SEQ ID NO 1206) |
| | | QNGVLNSWTDQDSK |
| | | (SEQ ID NO 1207) |
| | | SSQSLVHSNGNTYLHWYLQKPGQSPK |
| | | (SEQ ID NO 1208) |
| | | TSTSPIVKSFNR |
| | | (SEQ ID NO 1209) |
| | | VSNRFSGVPDR |
| | | (SEQ ID NO 1210) |
| gi\|2624743 | Ig (L Chain L, | DEYERHNSYTCEATHK cam |
| | Structure Of A | (SEQ ID NO 1211) |
| | Catalytic Antibody, | DIVMTQSPLTLSVTIGQPASISCKSSQSLLYSNGK ox |
| | Igg2a Fab Fragment) | cam |
| | | (SEQ ID NO 1212) |
| | | DSTYSMSSTLTLTKDEYER |
| | | (SEQ ID NO 1213) |
| | | DSTYSMSSTLTLTKDEYER ox |
| | | (SEQ ID NO 1214) |
| | | FTGSGSGTDFTLK |
| | | (SEQ ID NO 1215) |
| | | HNSYTCEATHK cam |
| | | (SEQ ID NO 1216) |
| | | LDSGVPDRFTGSGSGTDFTLK |
| | | (SEQ ID NO 1217) |
| | | QNGVLNSWTDQDSK |
| | | (SEQ ID NO 1218) |
| gi\|32263981 | Ig (mAb | FSGSGSGTSYSLTISR |
| | immunoglobulin light | (SEQ ID NO 1219) |
| | chain variable region) | FSGSGSGTSYSLTISRMEAEDAATYYCQQR ox cam |
| | | (SEQ ID NO 1220) |
| | | MEAEDAATYYCQQR cam |
| | | (SEQ ID NO 1221) |
| | | MEAEDAATYYCQQR ox cam |
| | | (SEQ ID NO 1222) |
| | | QIVLTQSPAIMSASPGEK ox |
| | | (SEQ ID NO 1223) |
| | | SSYPFTFGSGTK |
| | | (SEQ ID NO 1224) |
| | | SSYPFTFGSGTKLEIK |
| | | (SEQ ID NO 1225) |
| gi\|18568342 | Ig (monoclonal | CDIQMTQTTSALSASLGDR cam |
| | antibody BBK-2 light | (SEQ ID NO 1226) |
| | chain) | CDIQMTQTTSALSASLGDR cam ox |
| | | (SEQ ID NO 1227) |
| | | DSTYSMSSTLTLTKDEYER ox |
| | | (SEQ ID NO 1228) |
| | | HNSYTCEATHK cam |
| | | (SEQ ID NO 1229) |
| | | HNSYTCEATHKTSTSPIVK cam |
| | | (SEQ ID NO 1230) |
| | | MRFSAQFLGLLLLCFQGTR ox cam |
| | | (SEQ ID NO 1231) |
| | | NLEQEDVATYFCQQGYTLPYTFGGGTKLEIK cam |
| | | (SEQ ID NO 1232) |
| | | QNGVLNSWTDQDSK |
| | | (SEQ ID NO 1233) |
| gi\|52382 | Ig (mu(b) | DGKLVESGFTTDPVTIENK |
| | immunoglobulin | (SEQ ID NO 1234) |
| | heavy chain) | DLHVPIPAVAEMNPNVNVFVPPR ox |
| | | (SEQ ID NO 1235) |
| | | EFVCTVTHR cam |
| | | (SEQ ID NO 1236) |
| | | EQLNLRESATVTCLVK cam |
| | | (SEQ ID NO 1237) |
| | | FISKPNEVHK |
| | | (SEQ ID NO 1238) |
| | | GFSPADISVQWLQR |
| | | (SEQ ID NO 1239) |
| | | GVASVCVEDWNNR cam |
| | | (SEQ ID NO 1240) |
| | | GVASVCVEDWNNRK cam |
| | | (SEQ ID NO 1241) |
| | | HPPAVYLLPPAR |
| | | (SEQ ID NO 1242) |
| | | KEFVCTVTHR cam |
| | | (SEQ ID NO 1243) |
| | | LICEATNFTPKPITVSWLK cam |
| | | (SEQ ID NO 1244) |
| | | LVESGFTTDPVTIENK |
| | | (SEQ ID NO 1245) |
| | | LVESGFTTDPVTIENKGSTPQTYK |
| | | (SEQ ID NO 1246) |
| | | NKDLHVPIPAVAEMNPNVNVFVPPR |
| | | (SEQ ID NO 1247) |
| | | NKDLHVPIPAVAEMNPNVNVFVPPR ox |
| | | (SEQ ID NO 1248) |
| | | NLVAMGCLAR cam |
| | | (SEQ ID NO 1249) |
| | | NLVAMGCLAR ox cam |
| | | (SEQ ID NO 1250) |
| | | SILEGSDEYLVCK cam |
| | | (SEQ ID NO 1251) |
| | | TGGKYLATSQVLLSPK |
| | | (SEQ ID NO 1252) |
| | | YLATSQVLLSPK |
| | | (SEQ ID NO 1253) |
| gi\|7439167 | Ig (PC4436 | APQVYTIPPPKEQMAK |
| | monoclonal antibody | (SEQ ID NO 1254) |
| | 13-1 heavy chain) | APQVYTIPPPKEQMAK ox |
| | | (SEQ ID NO 1255) |
| | | CRVNSAAFPAPIEK cam |
| | | (SEQ ID NO 1256) |
| | | DCGCKPCICTVPEVSSVFIFPPKPK cam cam cam |
| | | cam |
| | | (SEQ ID NO 1257) |
| | | DDSKSSVYLQMNR |
| | | (SEQ ID NO 1258) |
| | | GRPKAPQVYTIPPPK |
| | | (SEQ ID NO 1259) |
| | | SNWEAGNTFTCSVLHEGLHNHHTEK cam |
| | | (SEQ ID NO 1260) |
| | | TTPPSVYPLAPGSAAQTNSMVTLGCLVK ox cam |
| | | (SEQ ID NO 1261) |
| | | VNSAAFPAPIEK |
| | | (SEQ ID NO 1262) |
| | | VNSAAFPAPIEKTISK |
| | | (SEQ ID NO 1263) |
| gi\|15824610 | Ig (Pterin-mimicking | DIVLTQSPASLAVSLGQR |
| | anti-idiotope kappa | (SEQ ID NO 1264) |
| | chain variable region) | FSGSGSGTDFTLNIHPVEEEDAATYYCQHSR cam |
| | | (SEQ ID NO 1265) |
| | | LLIYLASNLESGVPAR |
| | | (SEQ ID NO 1266) |
| | | ELPYTFGGGTKLEIK |
| | | (SEQ ID NO 1267) |
| | | DIVLTQSPASLAVSLGQRATISCR cam |
| | | (SEQ ID NO 68) |
| gi\|780265 | Ig gamma-1 chain C | APQVYTIPPPKEQMAK ox |
| | region (15C5) | (SEQ ID NO 1269) |
| | (fragment) | CRVNSAAFPAPIEK cam |
| | | (SEQ ID NO 1270) |
| | | NTQPIMDTDGSYFVYSKLNVQK ox |
| | | (SEQ ID NO 1271) |
| | | SNWEAGNTFTCSVLHEGLHNHHTEK cam |
| | | (SEQ ID NO 1272) |
| | | SVSELPIMHQDWLNGK |
| | | (SEQ ID NO 1273) |
| | | SVSELPIMHQDWLNGK ox |
| | | (SEQ ID NO 1274) |
| | | VNSAAFPAPIEK |
| | | (SEQ ID NO 1275) |
| | | VNSAAFPAPIEKTISK |
| | | (SEQ ID NO 1276) |
| gi\|1799551 | Ig gamma-chain, | AQTPQVYTIPPPR |
| | secrete-type | (SEQ ID NO 1277) |
| | | AQTPQVYTIPPPREQMSK |
| | | (SEQ ID NO 1278) |
| | | AQTPQVYTIPPPREQMSK ox |
| | | (SEQ ID NO 1279) |
| | | DALMISLTPK ox |
| | | (SEQ ID NO 1280) |
| | | EVHTAWTQPR |
| | | (SEQ ID NO 1281) |
| | | GYFPEPVTVK |
| | | (SEQ ID NO 1282) |
| | | GYFPEPVTVKWNYGALSSGVR |
| | | (SEQ ID NO 1283) |
| | | NTPPILDSDGTYFLYSK |
| | | (SEQ ID NO 1284) |
| | | VNNKALPAPIER |
| | | (SEQ ID NO 1285) |
| | | VVSALPIQHQDWMR |
| | | (SEQ ID NO 1286) |
| | | VVSALPIQHQDWMR ox |
| | | (SEQ ID NO 1287) |
| | | WNYGALSSGVR |
| | | (SEQ ID NO 1288) |
| gi\|223428 | Ig H-C allotype | APQVYILPPPAEQLSR |
| | gamma2b | (SEQ ID NO 1289) |
| | | APQVYILPPPAEQLSRK |
| | | (SEQ ID NO 1290) |
| | | CPAPNLEGGPSVFIFPPNIK cam |
| | | (SEQ ID NO 1291) |
| | | CPAPNLEGGPSVFIFPPNIKDVLMISLTPK cam |
| | | (SEQ ID NO 1292) |
| | | CPAPNLEGGPSVFIFPPNIKDVLMISLTPK cam ox |
| | | (SEQ ID NO 1293) |
| | | GLVRAPQVYILPPPAEQLSR |
| | | (SEQ ID NO 1294) |
| | | HEGLKNYYLK |
| | | (SEQ ID NO 1295) |
| | | ISWFVNNVEVHTAQTQTHR |
| | | (SEQ ID NO 1296) |
| | | LEPSGPISTINPCPPCKECHK cam cam cam |
| | | (SEQ ID NO 1297) |
| | | TDSFSCNVR cam |
| | | (SEQ ID NO 1298) |
| | | TDSFSCNVRHEGLK cam |
| | | (SEQ ID NO 1299) |
| | | TTPPSVYPLAPGCGDTTGSSVTLGCLVK cam cam |
| | | (SEQ ID NO 1300) |
| | | VNNKDLPSPIER |
| | | (SEQ ID NO 1301) |
| | | VTCVVVDVSEDDPDVR cam |
| | | (SEQ ID NO 1302) |
| | | VVSALPIQHQDWMSGK |
| | | (SEQ ID NO 1303) |
| | | VVSALPIQHQDWMSGK ox |
| | | (SEQ ID NO 1304) |
| | | VVSALPIQHQDWMSGKEFK |
| | | (SEQ ID NO 1305) |
| | | VVSALPIQHQDWMSGKEFK o |
| | | (SEQ ID NO 1306) |
| | | WEKTDSFSCNVR cam |
| | | (SEQ ID NO 1307) |
| gi\|224008 | Ig J chain | CYTTMVPLR cam |
| | | (SEQ ID NO 1308) |
| | | CYTTMVPLR cam ox |
| | | (SEQ ID NO 1309) |
| | | CYTTMVPLRYHGETK cam |
| | | (SEQ ID NO 1310) |
| | | CYTTMVPLRYHGETK cam ox |
| | | (SEQ ID NO 1311) |
| | | IIPSTEDPNEDIVER |
| | | (SEQ ID NO 1312) |
| | | IIPSTEDPNEDIVERNIR |
| | | (SEQ ID NO 1313) |
| | | MVQAALTPDSCYPD ox cam |
| | | (SEQ ID NO 1314) |
| | | NFVYHLSDVCK cam |
| | | (SEQ ID NO 1315) |
| | | NFVYHLSDVCKK cam |
| | | (SEQ ID NO 1316) |
| gi\|12832551 | Ig K (unnamed | ADAAPTVSIFPPSSEQLTSGGASVVCFLNNFYPK |
| | protein product) | cam |
| | | (SEQ ID NO 1317) |
| | | ASENIYSNLAWYQQK |
| | | (SEQ ID NO 1318) |
| | | ASENIYSNLAWYQQKQGK |
| | | (SEQ ID NO 1319) |
| | | HNSYTCEATHK cam |
| | | (SEQ ID NO 1320) |
| | | HNSYTCEATHKTSTSPIVK cam |
| | | (SEQ ID NO 1321) |
| | | QGKSPQLLVYAATNLADGVPSR |
| | | (SEQ ID NO 1322) |
| | | RADAAPTVSIFPPSSEQLTSGGASVVCFLNNFYPK |
| | | cam |
| | | (SEQ ID NO 1323) |
| | | SPQLLVYAATNLADGVPSR |
| | | (SEQ ID NO 1324) |
| gi\|896077 | Ig kappa chain | ASSSVSSSYLHWYQQK |
| | | (SEQ ID NO 1325) |
| | | ENVLTQSPAIMSASPGEK ox |
| | | (SEQ ID NO 1326) |
| | | ENVLTQSPAIMSASPGEKVTMTCR ox cam |
| | | (SEQ ID NO 1327) |
| | | ENVLTQSPAIMSASPGEKVTMTCR ox ox cam |
| | | (SEQ ID NO 1328) |
| | | LWIYSTSNLASGVPAR |
| | | (SEQ ID NO 1329) |
| | | SGASPKLWIYSTSNLASGVPAR |
| | | (SEQ ID NO 1330) |
| gi\|896089 | Ig kappa chain | DIVMSQSPSSLAVSVGEK ox |
| | | (SEQ ID NO 1331) |
| | | ESGVPDRFTGSGSGTDFTLTISSVK |
| | | (SEQ ID NO 1332) |
| | | FTGSGSGTDFTLTISSVK |
| | | (SEQ ID NO 1333) |
| | | LLIYWASTR |
| | | (SEQ ID NO 1334) |
| | | NYLAWYQQKPGQSPK |
| | | (SEQ ID NO 1335) |
| | | SSQSLLYSSNQK |
| | | (SEQ ID NO 1336) |
| gi\|896103 | Ig kappa chain | ASQDIGSSLNWLQQEPDGTIKR |
| | | (SEQ ID NO 1337) |
| | | DIQMTQSPSSLSASLGER |
| | | (SEQ ID NO 1338) |
| | | DIQMTQSPSSLSASLGER ox |
| | | (SEQ ID NO 1339) |
| | | LIYATSSLDSGVPK |
| | | (SEQ ID NO 1340) |
| | | LIYATSSLDSGVPKR |
| | | (SEQ ID NO 1341) |
| gi\|896107 | Ig kappa chain | ADAAPTVSIFPPSSEQLTSGGASVVCFLNNFYPK |
| | | cam |
| | | (SEQ ID NO 1342) |
| | | ASENIYSNLAWYQQK |
| | | (SEQ ID NO 1343) |
| | | ASENIYSNLAWYQQKQGK |
| | | (SEQ ID NO 1344) |
| | | HNSYTCEATHK cam |
| | | (SEQ ID NO 1345) |
| | | HNSYTCEATHKTSTSPIVK cam |
| | | (SEQ ID NO 1346) |
| | | QGKSPQLLVYAATNLADGVPSR |
| | | (SEQ ID NO 1347) |
| | | RADAAPTVSIFPPSSEQLTSGGASVVCFLNNFYPK |
| | | cam |
| | | (SEQ ID NO 1348) |
| | | SPQLLVYAATNLADGVPSR |
| | | (SEQ ID NO 1349) |
| gi\|7513693 | Ig kappa chain | ADAAPTVSIFPPSSEQLTSGGASVVCFLNNFYPK |
| | (Mab03-1) | cam |
| | | (SEQ ID NO 1350) |
| | | DEYERHNSYTCEATHK cam |
| | | (SEQ ID NO 1351) |
| | | DIVMTQSPSSLAMSVGQK |
| | | (SEQ ID NO 1352) |
| | | DIVMTQSPSSLAMSVGQK ox |
| | | (SEQ ID NO 1353) |
| | | DIVMTQSPSSLAMSVGQK ox ox |
| | | (SEQ ID NO 1354) |
| | | DSTYSMSSTLTLTKDEYER ox |
| | | (SEQ ID NO 1355) |
| | | HNSYTCEATHK cam |
| | | (SEQ ID NO 1356) |
| | | HNSYTCEATHKTSTSPIVK cam |
| | | (SEQ ID NO 1357) |
| | | LLVYFASTR |
| | | (SEQ ID NO 1358) |
| | | NYLAWYQQKPGQSPK |
| | | (SEQ ID NO 1359) |
| | | SSQSLLNSRNQK |
| | | (SEQ ID NO 1360) |
| gi\|110429 | Ig kappa chain V | ASQDINKYIAWYQHKPGK |
| | region (9.42) | (SEQ ID NO 1361) |
| | | DIQMTQSPSSLSASLGGK |
| | | (SEQ ID NO 1362) |
| | | DIQMTQSPSSLSASLGGK ox |
| | | (SEQ ID NO 1363) |
| | | LLIHYTSTLQPGIPSR |
| | | (SEQ ID NO 1364) |
| | | YIAWYQHKPGK |
| | | (SEQ ID NO 1365) |
| | | YIAWYQHKPGKGPR |
| | | (SEQ ID NO 1366) |
| gi\|49258884 | Ig kappa chain V | ADAAPTVSIFPPSSEQLTSGGASVVCFLNNFYPK |
| | region (D444) | cam |
| | | (SEQ ID NO 1367) |
| | | ASQSIGTDIHWYQQR |
| | | (SEQ ID NO 1368) |
| | | DILLTQSPAILSVSPGER |
| | | (SEQ ID NO 1369) |
| | | HNSYTCEATHK cam |
| | | (SEQ ID NO 1370) |
| | | LLIKYASESISGIPSR |
| | | (SEQ ID NO 1371) |
| | | RADAAPTVSIFPPSSEQLTSGGASVVCFLNNFYPK |
| | | cam |
| | | (SEQ ID NO 1372) |
| | | YASESISGIPSR |
| | | (SEQ ID NO 1373) |
| gi\|930226 | Ig kappa chain V | ASQDIKSYLSWYQQKPWK |
| | region (hybridoma | (SEQ ID NO 1374) |
| | NC19-E11) | DIKMTQSPSSMYASLGER ox |
| | | (SEQ ID NO 1375) |
| | | DIKMTQSPSSMYASLGER ox ox |
| | | (SEQ ID NO 1376) |
| | | MTQSPSSMYASLGER ox |
| | | (SEQ ID NO 1377) |
| | | MTQSPSSMYASLGER ox ox |
| | | (SEQ ID NO 1378) |
| | | SYLSWYQQKPWK |
| | | (SEQ ID NO 1379) |
| | | SYLSWYQQKPWKSPK |
| | | (SEQ ID NO 1380) |
| | | TLIYYATSLADGVPSR |
| | | (SEQ ID NO 1381) |
| gi\|125796 | Ig kappa chain V-III | ASESVDNYGISFMNWFQQKPGQPPK |
| | region PC 2880/PC | (SEQ ID NO 1382) |
| | 1229 | ASESVDNYGISFMNWFQQKPGQPPK ox |
| | | (SEQ ID NO 1383) |
| | | DIVLTQSPASLAVSLGQR |
| | | (SEQ ID NO 1384) |
| | | EVPWTFGGGTK |
| | | (SEQ ID NO 1385) |
| | | EVPWTFGGGTKLEIK |
| | | (SEQ ID NO 1386) |
| | | LLIYAASNQGSGVPAR |
| | | (SEQ ID NO 1387) |
| gi\|197064 | Ig kappa chain V- | ASQDISNYLNWYQQKPDGTVK |
| | region (V-Jk1) protein | (SEQ ID NO 1388) |
| | | DIQMTQTTSSLSASLGDR ox |
| | | (SEQ ID NO 1389) |
| | | DIQMTQTTSSLSASLGDRVTISCR cam |
| | | (SEQ ID NO 1390) |
| | | DIQMTQTTSSLSASLGDRVTISCR ox cam |
| | | (SEQ ID NO 1391) |
| | | FSGSGSGTDYSLTISNLEQEDIATYFCQQGNTLPR |
| | | cam |
| | | (SEQ ID NO 1392) |
| | | LLIYYTSRLHSGVPSR |
| | | (SEQ ID NO 1393) |
| | | TFGGGTKLEIK |
| | | (SEQ ID NO 1394) |
| gi\|1613779 | Ig kappa light chain | ADAAPTVSIFPPSSEQLTSGGASVVCFLNNFYPK |
| | | cam |
| | | (SEQ ID NO 1395) |
| | | ANRLVDGVPSR |
| | | (SEQ ID NO 1396) |
| | | DIKMTQSPSSMYASLGER |
| | | (SEQ ID NO 1397) |
| | | DIKMTQSPSSMYASLGER ox |
| | | (SEQ ID NO 1398) |
| | | DIKMTQSPSSMYASLGER ox ox |
| | | (SEQ ID NO 1399) |
| | | HNSYTCEATHK cam |
| | | (SEQ ID NO 1400) |
| | | HNSYTCEATHKTSTSPIVK cam |
| | | (SEQ ID NO 1401) |
| | | MTQSPSSMYASLGER |
| | | (SEQ ID NO 1402) |
| | | MTQSPSSMYASLGER ox |
| | | (SEQ ID NO 1403) |
| | | MTQSPSSMYASLGER ox ox |
| | | (SEQ ID NO 1404) |
| gi\|284924 | Ig light chain V region | ASENIYSYLAWYQQK |
| | (clone 185-c1) | (SEQ ID NO 1405) |
| | | DIQMTQSPASLSASVGETVTITCR ox cam |
| | | (SEQ ID NO 6 |
| | | FSGSGSGTQFSLK |
| | | (SEQ ID NO 1407) |
| | | QGKSPQLLVYNAK |
| | | (SEQ ID NO 1408) |
| | | SPQLLVYNAK |
| | | (SEQ ID NO 1409) |
| | | TLAEGVPSRFSGSGSGTQFSLK |
| | | (SEQ ID NO 1410) |
| gi\|494164 | Igg2a Fab Fragment | ADAAPTVSIFPPSSEQLTSGGASVVCFLNNFYPK |
| | (Fab 179) | cam |
| | | (SEQ ID NO 1411) |
| | | DIVMTQSPSSLTVTAGEK ox |
| | | (SEQ ID NO 1412) |
| | | HNSYTCEATHK cam |
| | | (SEQ ID NO 1413) |
| | | HNSYTCEATHKTSTSPIVK cam |
| | | (SEQ ID NO 1414) |
| | | NYLTWYQQKPGQPPK |
| | | (SEQ ID NO 1415) |
| gi\|229901 | Igg2b, Kappa | ADAAPTVSIFPPSSEQLTSGGASVVCFLNNFYPK |
| | | cam |
| | | (SEQ ID NO 1416) |
| | | ASQDISNYLNWYQQKPDGTVK |
| | | (SEQ ID NO 1417) |
| | | DIQMTQTTSSLSASLGDR |
| | | (SEQ ID NO 1418) |
| | | DIQMTQTTSSLSASLGDR ox |
| | | (SEQ ID NO 1419) |
| | | DIQMTQTTSSLSASLGDRVTISCR cam |
| | | (SEQ ID NO 1420) |
| | | DIQMTQTTSSLSASLGDRVTISCR ox cam |
| | | (SEQ ID NO 1421) |
| | | DSTYSMSSTLTLTKDEYER ox |
| | | (SEQ ID NO 1422) |
| | | HNSYTCEATHK cam |
| | | (SEQ ID NO 1423) |
| | | HNSYTCEATHKTSTSPIVK cam |
| | | (SEQ ID NO 1424) |
| | | LLIYYTSR |
| | | (SEQ ID NO 1425) |
| | | QNGVLNSWTDQDSK |
| | | (SEQ ID NO 1426) |
| | | TFGGGTKLEIK |
| | | (SEQ ID NO 1427) |
| gi\|62204734 | Inter alpha-trypsin | ADAVQEATFQVELPR |
| | inhibitor, heavy chain | (SEQ ID NO 1428) |
| | 4 | AHGGTNINNAVLLAVELLDR |
| | | (SEQ ID NO 1429) |
| | | AHGGTNINNAVLLAVELLDRSNQAELLPSK |
| | | (SEQ ID NO 1430) |
| | | DIVWEPPVEPDNTKR |
| | | (SEQ ID NO 1431) |
| | | FKPTLSQQQK |
| | | (SEQ ID NO 1432) |
| | | GSEMVVAGKLQDQGPDVLLAK ox |
| | | (SEQ ID NO 1433) |
| | | ILKDLSPQDQFNLIEFSGEANQWK |
| | | (SEQ ID NO 1434) |
| | | IRAHGGTNINNAVLLAVELLDR |
| | | (SEQ ID NO 1435) |
| | | ITFELIYQELLQR |
| | | (SEQ ID NO 1436) |
| | | ITFELIYQELLQRR |
| | | (SEQ ID NO 1437) |
| | | LFVDPSQGLEVTGK |
| | | (SEQ ID NO 1438) |
| | | LPLAAQAHPFRPPVR |
| | | (SEQ ID NO 1439) |
| | | LWALLTIQQQLEQR |
| | | (SEQ ID NO 1440) |
| | | MLSLPSVAQYPADPHLVVTEK ox |
| | | (SEQ ID NO 1441) |
| | | NTEYKWK |
| | | (SEQ ID NO 1442) |
| | | QSLVQATEENLNKAVNYASR |
| | | (SEQ ID NO 1443) |
| | | QTEKFEVSVNVAPGSK |
| | | (SEQ ID NO 1444) |
| | | QYSAAVGRGESAGIVK |
| | | (SEQ ID NO 1445) |
| | | RLGMYELLLK |
| | | (SEQ ID NO 1446) |
| | | RLGMYELLLK ox |
| | | (SEQ ID NO 1447) |
| | | SQSEQDTVLNGDFIVR |
| | | (SEQ ID NO 1448) |
| | | SVSLIILLTDGDPTVGETNPTIIQNNVR |
| | | (SEQ ID NO 1449) |
| | | TLFSVLPGLK |
| | | (SEQ ID NO 1450) |
| | | TVKVQGVDYLATR |
| | | (SEQ ID NO 1451) |
| | | VQGVDYLATR |
| | | (SEQ ID NO 1452) |
| | | VVNRADAVQEATFQVELPR |
| | | (SEQ ID NO 1453) |
| | | YIFHNFMER |
| | | (SEQ ID NO 1454) |
| | | YIFHNFMER ox |
| | | (SEQ ID NO 1455) |
| | | YKFQHHFK |
| | | (SEQ ID NO 1456) |
| gi\|16418335 | Leucine-rich alpha-2- | CAGPEAMKGQR cam |
| | glycoprotein | (SEQ ID NO 1457) |
| | | CAGPEAMKGQR cam ox |
| | | (SEQ ID NO 1458) |
| | | DMQDGFDISHNPWICDKNLADLCR ox cam cam |
| | | (SEQ ID NO 1459) |
| | | ELHLSSNRLQALSPELLAPVPR |
| | | (SEQ ID NO 1460) |
| | | LEDSLLAPQPFLR |
| | | (SEQ ID NO 1461) |
| | | LHLEGNRLQR |
| | | (SEQ ID NO 1462) |
| | | LQALSPELLAPVPR |
| | | (SEQ ID NO 1463) |
| | | LQRLEDSLLAPQPFLR |
| | | (SEQ ID NO 1464) |
| gi\|21594125 | Lifr protein (leukemia | AEIQLSKNDYIISVVAR |
| | inhibitory factor | (SEQ ID NO 1465) |
| | receptor) | GAEDSTYHVAVDKLNPYTAYTFR |
| | | (SEQ ID NO 1466) |
| | | HLTTEATPSKGPDTWR |
| | | (SEQ ID NO 1467) |
| | | IASMEIPNDDITVEQAVGLGNR ox |
| | | (SEQ ID NO 1468) |
| | | INLLCQIEICK cam cam |
| | | (SEQ ID NO 1469) |
| | | LSCETHDLKEIICSWNPGR cam cam |
| | | (SEQ ID NO 1470) |
| | | NDYIISVVAR |
| | | (SEQ ID NO 1471) |
| | | NTEYTLFESISGK |
| | | (SEQ ID NO 1472) |
| | | SEPCLLDWR cam |
| | | (SEQ ID NO 1473) |
| | | YNFYLYGCTNQGYQLLR cam |
| | | (SEQ ID NO 1474) |
| gi\|8569601 | Major urinary protein | AGEYSVTYDGFNTFTIPK |
| | 1 | (SEQ ID NO 1475) |
| | | DGETFQLMGLYGR ox |
| | | (SEQ ID NO 1476) |
| | | EEASSTGRNFNVEK |
| | | (SEQ ID NO 1477) |
| | | EKIEDNGNFR |
| | | (SEQ ID NO 1478) |
| | | ENIIDLSNANR |
| | | (SEQ ID NO 1479) |
| | | FAQLCEEHGILR cam |
| | | (SEQ ID NO 1480) |
| | | FAQLCEEHGILRENIIDLSNANR cam |
| | | (SEQ ID NO 1481) |
| | | IEDNGNFRLFLEQIHVLEK |
| | | (SEQ ID NO 1482) |
| | | INGEWHTIILASDKR |
| | | (SEQ ID NO 1483) |
| | | LFLEQIHVLEK |
| | | (SEQ ID NO 1484) |
| | | TDYDNFLMAHLINEK ox |
| | | (SEQ ID NO 1485) |
| gi\|6754654 | Mannose binding | AIEEKLANMEAEIR |
| | lectin (A) | (SEQ ID NO 1486) |
| | | AIEEKLANMEAEIR ox |
| | | (SEQ ID NO 1487) |
| | | AIQEVATGIAFLGITDEATEGQFMYVTGGR ox |
| | | (SEQ ID NO 1488) |
| | | GEPGQGLRGLQGPPGK |
| | | (SEQ ID NO 1489) |
| | | LANMEAEIR ox |
| | | (SEQ ID NO 1490) |
| | | LFVTNHEKMPFSK ox |
| | | (SEQ ID NO 1491) |
| | | LQLTNKLHAFSMGK ox |
| | | (SEQ ID NO 1492) |
| | | SLCTELQGTVAIPR cam |
| | | (SEQ ID NO 1493) |
| | | SLCTELQGTVAIPRNAEENK cam |
| | | (SEQ ID NO 1494) |
| | | VKSLCTELQGTVAIPR cam |
| | | (SEQ ID NO 1495) |
| gi\|33018 | Mannose binding | AEFDTSEIDSEIAALR |
| | protein C | (SEQ ID NO 1496) |
| | | AEFDTSEIDSEIAALRSELR |
| | | (SEQ ID NO 1497) |
| | | ALCSEFQGSVATPR cam |
| | | (SEQ ID NO 1498) |
| | | ALRNWVLFSLSEK |
| | | (SEQ ID NO 1499) |
| | | DIAYLGITDVR |
| | | (SEQ ID NO 1500) |
| | | DIAYLGITDVRVEGSFEDLTGNR |
| | | (SEQ ID NO 1501) |
| | | NWVLFSLSEK |
| | | (SEQ ID NO 1502) |
| | | VAKDIAYLGITDVR |
| | | (SEQ ID NO 1503) |
| | | VEGSFEDLTGNR |
| | | (SEQ ID NO 1504) |
| | | VEGSFEDLTGNRVR |
| | | (SEQ ID NO 1505) |
| | | VKALCSEFQGSVATPR cam |
| | | (SEQ ID NO 1506) |
| | | WNDVPCSDSFLAICEFSD cam cam |
| | | (SEQ ID NO 1507) |
| | | YTNWNDGEPNNTGDGEDCVVILGNGK cam |
| | | (SEQ ID NO 1508) |
| gi\|2118830 | MHC class I | AKGNEQSFHVSLR |
| | histocompatibility | (SEQ ID NO 1509) |
| | antigen H-2 Q10 | APWMEQEGPEYWER |
| | alpha chain | (SEQ ID NO 1510) |
| | | APWMEQEGPEYWER ox |
| | | (SEQ ID NO 1511) |
| | | APWMEQEGPEYWERETQR |
| | | (SEQ ID NO 1512) |
| | | APWMEQEGPEYWERETQR ox |
| | | (SEQ ID NO 1513) |
| | | AYLEAECVEWLLR cam |
| | | (SEQ ID NO 1514) |
| | | FDSDAETPRMEPR |
| | | (SEQ ID NO 1515) |
| | | FDSDAETPRMEPR ox |
| | | (SEQ ID NO 1516) |
| | | FIIVGYVDDTQFVR |
| | | (SEQ ID NO 1517) |
| | | FIIVGYVDDTQFVRFDSDAETPR |
| | | (SEQ ID NO 1518) |
| | | GNEQSFHVSLR |
| | | (SEQ ID NO 1519) |
| | | GYLQYAYDGR |
| | | (SEQ ID NO 1520) |
| | | GYLQYAYDGRDYIALNEDLK |
| | | (SEQ ID NO 1521) |
| | | KWEQAGAAEYYR |
| | | (SEQ ID NO 1522) |
| | | TDPPKTHVTHHPGSEGDVTLR |
| | | (SEQ ID NO 1523) |
| | | THVTHHPGSEGDVTLR |
| | | (SEQ ID NO 1524) |
| | | TWTAADVAAIITR |
| | | (SEQ ID NO 1525) |
| | | WASVVVPLGK |
| | | (SEQ ID NO 1526) |
| | | WEQAGAAEYYR |
| | | (SEQ ID NO 1527) |
| | | YFETSVSRPGLGEPR |
| | | (SEQ ID NO 1528) |
| | | YLELGKETLLR |
| | | (SEQ ID NO 1529) |
| gi\|387437 | MHC factor B | AEGIPEFYDYDVALVK |
| | | (SEQ ID NO 1530) |
| | | ALFVSEQGK |
| | | (SEQ ID NO 1531) |
| | | ALLDIGRDPK |
| | | (SEQ ID NO 1532) |
| | | ALRLPQTATCK cam |
| | | (SEQ ID NO 1533) |
| | | CLTNLIEK cam |
| | | (SEQ ID NO 1534) |
| | | DNEHHVFK |
| | | (SEQ ID NO 1535) |
| | | FIQVGVISWGVVDVCR cam |
| | | (SEQ ID NO 1536) |
| | | FLCTGGVDPYADPNTCK cam cam |
| | | (SEQ ID NO 1537) |
| | | GDSGGPLIVHKR |
| | | (SEQ ID NO 1538) |
| | | GNDYHKQPWQAK |
| | | (SEQ ID NO 1539) |
| | | HVIIIMTDGLHNMGGNPVTVIQDIR ox |
| | | (SEQ ID NO 1540) |
| | | HVIIIMTDGLHNMGGNPVTVIQDIR ox ox |
| | | (SEQ ID NO 1541) |
| | | ISVTRPLK |
| | | (SEQ ID NO 1542) |
| | | KAEGIPEFYDYDVALVK |
| | | (SEQ ID NO 1543) |
| | | KDNEHHVFK |
| | | (SEQ ID NO 1544) |
| | | LKDEDLGFL |
| | | (SEQ ID NO 1545) |
| | | LKYGQTLRPICLPCTEGTTR cam cam |
| | | (SEQ ID NO 1546) |
| | | LNQISYEDHKLK |
| | | (SEQ ID NO 1547) |
| | | RCLTNLIEK cam |
| | | (SEQ ID NO 1548) |
| | | RDLEIEEVLFHPK |
| | | (SEQ ID NO 1549) |
| | | SLSLCGMVWEHK cam |
| | | (SEQ ID NO 1550) |
| | | SLSLCGMVWEHK cam ox |
| | | (SEQ ID NO 1551) |
| | | SLSLCGMVWEHKK cam |
| | | (SEQ ID NO 1552) |
| | | SLSLCGMVWEHKK cam ox |
| | | (SEQ ID NO 1553) |
| | | VASYGVRPR |
| | | (SEQ ID NO 1554) |
| | | VKDASEVVTPR |
| | | (SEQ ID NO 1555) |
| | | VKDMEDLENVFYQMIDETK ox ox |
| | | (SEQ ID NO 1556) |
| | | VSVGGQRR |
| | | (SEQ ID NO 1557) |
| | | YGLLTYATVPK |
| | | (SEQ ID NO 1558) |
| | | YGQTLRPICLPCTEGTTR cam cam |
| | | (SEQ ID NO 1559) |
| gi\|55217 | Murine valosin- | EAVCIVLSDDTCSDEKIR cam cam |
| | containing protein | (SEQ ID NO 1560) |
| | | ELQELVQYPVEHPDKFLK |
| | | (SEQ ID NO 1561) |
| | | EVDIGIPDATGRLEILQIHTK |
| | | (SEQ ID NO 1562) |
| | | GILLYGPPGTGK |
| | | (SEQ ID NO 1563) |
| | | GVLFYGPPGCGK cam |
| | | (SEQ ID NO 1564) |
| | | IVSQLLTLMDGLK |
| | | (SEQ ID NO 1565) |
| | | IVSQLLTLMDGLKQR |
| | | (SEQ ID NO 1566) |
| | | IVSQLLTLMDGLKQR ox |
| | | (SEQ ID NO 1567) |
| | | KYEMFAQTLQQSR ox |
| | | (SEQ ID NO 1568) |
| | | MTNGFSGADLTEICQR ox cam |
| | | (SEQ ID NO 1569) |
| | | QAAPCVLFFDELDSIAK cam |
| | | (SEQ ID NO 1570) |
| | | WALSQSNPSALR |
| | | (SEQ ID NO 1571) |
| gi\|6679182 | Orosomucoid 1 | AVTHVGMDESEIIFVDWK ox |
| | | (SEQ ID NO 1572) |
| | | AVTHVGMDESEIIFVDWKK |
| | | (SEQ ID NO 1573) |
| | | AVTHVGMDESEIIFVDWKK ox |
| | | (SEQ ID NO 1574) |
| | | HGAFMLAFDLKDEK |
| | | (SEQ ID NO 1575) |
| | | HGAFMLAFDLKDEK ox |
| | | (SEQ ID NO 1576) |
| | | RPDITPELR |
| | | (SEQ ID NO 1577) |
| | | RPDITPELREVFQK |
| | | (SEQ ID NO 1578) |
| | | WFFMGAAFR |
| | | (SEQ ID NO 1579) |
| | | WFFMGAAFR ox |
| | | (SEQ ID NO 1580) |
| | | YEGGVETFAHLIVLR |
| | | (SEQ ID NO 1581) |
| | | YEGGVETFAHLIVLRK |
| | | (SEQ ID NO 1582) |
| gi\|31982113 | Plasminogen [Mus | CEGETDFVCR cam cam |
| | musculus] | (SEQ ID NO 1583) |
| | | CQSWAAMFPHR cam |
| | | (SEQ ID NO 1584) |
| | | CQSWAAMFPHR cam ox |
| | | (SEQ ID NO 1585) |
| | | CQSWAAMFPHRHSK cam |
| | | (SEQ ID NO 1586) |
| | | CQSWAAMFPHRHSK cam ox |
| | | (SEQ ID NO 1587) |
| | | CTTPPPPPSPTYQCLK cam cam |
| | | (SEQ ID NO 1588) |
| | | FTGQHFCGGTLIAPEWVLTAAHCLEK cam cam |
| | | (SEQ ID NO 1589) |
| | | FVDWIEREMR |
| | | (SEQ ID NO 1590) |
| | | FVDWIEREMR ox |
| | | (SEQ ID NO 1591) |
| | | GENYRGTVSVTVSGK |
| | | (SEQ ID NO 1592) |
| | | GKTAVTAAGTPCQGWAAQEPHR cam |
| | | (SEQ ID NO 1593) |
| | | HNRTPENFPCK cam |
| | | (SEQ ID NO 1594) |
| | | HSIFTPQTNPR |
| | | (SEQ ID NO 1595) |
| | | HSKTPENFPDAGLEMNYCR cam |
| | | (SEQ ID NO 1596) |
| | | HSKTPENFPDAGLEMNYCR ox cam |
| | | (SEQ ID NO 1597) |
| | | KCQSWAAMFPHR cam |
| | | (SEQ ID NO 1598) |
| | | KCQSWAAMFPHR cam ox |
| | | (SEQ ID NO 1599) |
| | | LILEPNNRDIALLK |
| | | (SEQ ID NO 1600) |
| | | LKEAQLPVIENK |
| | | (SEQ ID NO 1601) |
| | | LSRPATITDK |
| | | (SEQ ID NO 1602) |
| | | LSRPATITDKVIPACLPSPNYMVADR cam |
| | | (SEQ ID NO 1603) |
| | | LSRPATITDKVIPACLPSPNYMVADR cam ox |
| | | (SEQ ID NO 1604) |
| | | MRDVILFEK |
| | | (SEQ ID NO 1605) |
| | | MRDVILFEK ox |
| | | (SEQ ID NO 1606) |
| | | NLEENYCR cam |
| | | (SEQ ID NO 1607) |
| | | NLEENYCRNPDGETAPWCYTTDSQLR cam cam |
| | | (SEQ ID NO 1608) |
| | | NPDGDKGPWCYTTDPSVR cam |
| | | (SEQ ID NO 1609) |
| | | NPDGDVNGPWCYTTNPR cam |
| | | (SEQ ID NO 1610) |
| | | NPDGDVNGPWCYTTNPRK cam |
| | | (SEQ ID NO 1611) |
| | | NPDGEPRPWCFTTDPTK cam |
| | | (SEQ ID NO 1612) |
| | | NPDGEPRPWCFTTDPTKR cam |
| | | (SEQ ID NO 1613) |
| | | NPDNDEQGPWCYTTDPDKR cam |
| | | (SEQ ID NO 1614) |
| | | NYCRNPDGDVNGPWCYTTNPR cam cam |
| | | (SEQ ID NO 1615) |
| | | QLAAGGVSDCLAK cam |
| | | (SEQ ID NO 1616) |
| | | QLAAGGVSDCLAKCEGETDFVCR cam cam cam |
| | | (SEQ ID NO 1617) |
| | | RVYLSECK cam |
| | | (SEQ ID NO 1618) |
| | | SFQYHSKEQQCVIMAENSK cam |
| | | (SEQ ID NO 1619) |
| | | TAVTAAGTPCQGWAAQEPHR cam |
| | | (SEQ ID NO 1620) |
| | | TGIGNGYRGTMSR ox |
| | | (SEQ ID NO 1621) |
| | | TICYITGWGETQGTFGAGR cam |
| | | (SEQ ID NO 1622) |
| | | TPENFPCKNLEENYCR cam cam |
| | | (SEQ ID NO 1623) |
| | | TPENFPDAGLEMNYCR cam |
| | | (SEQ ID NO 1624) |
| | | TPENFPDAGLEMNYCR ox cam |
| | | (SEQ ID NO 1625) |
| | | VCNRVEYLNNR cam |
| | | (SEQ ID NO 1626) |
| | | VILGAHEEYIR |
| | | (SEQ ID NO 1627) |
| | | VILGAHEEYIRGSDVQEISVAK |
| | | (SEQ ID NO 1628) |
| | | VIPACLPSPNYMVADR cam |
| | | (SEQ ID NO 1629) |
| | | VIPACLPSPNYMVADR cam ox |
| | | (SEQ ID NO 1630) |
| | | VSRFVDWIER |
| | | (SEQ ID NO 1631) |
| | | WEYCDIPR cam |
| | | /SEQ ID NO 1632) |
| | | WEYCNLKR cam |
| | | (SEQ ID NO 1633) |
| | | WGATFPHVPNYSPSTHPNEGLEENYCR cam |
| | | (SEQ ID NO 1634) |
| | | WSEQTPHRHNR |
| | | (SEQ ID NO 1635) |
| | | YDYCNIPECEEECMYCSGEKYEGK cam cam cam ox |
| | | cam |
| | | (SEQ ID NO 1636) |
| gi\|2144495 | PLMS plasmin | CEGETDFVCR cam cam |
| | | (SEQ ID NO 1637) |
| | | CQSWAAMFPHR cam |
| | | (SEQ ID NO 1638) |
| | | CQSWAAMFPHR cam ox |
| | | (SEQ ID NO 1639) |
| | | CTTPPPPPSPTYQCLK cam cam |
| | | (SEQ ID NO 1640) |
| | | EQQCVIMAENSK cam ox |
| | | (SEQ ID NO 1641) |
| | | FTGQHFCGGTLIAPEWVLTAAHCLEK cam cam |
| | | (SEQ ID NO 1642) |
| | | FVDWIEREMR ox |
| | | (SEQ ID NO 1643) |
| | | GENYRGTVSVTVSGK |
| | | (SEQ ID NO 1644) |
| | | GKTAVTAAGTPCQGWAAQEPHR cam |
| | | (SEQ ID NO 1645) |
| | | HSIFTPQTNPR |
| | | (SEQ ID NO 1646) |
| | | KCQSWAAMFPHR cam |
| | | (SEQ ID NO 1647) |
| | | KCQSWAAMFPHR cam ox |
| | | (SEQ ID NO 1648) |
| | | LILEPNNRDIALLK |
| | | (SEQ ID NO 1649) |
| | | LKEAQLPVIENK |
| | | (SEQ ID NO 1650) |
| | | LSRPATITDK |
| | | (SEQ ID NO 1651) |
| | | LYDYCDIPLCASASSFECGKPQVEPKK cam cam |
| | | cam (SEQ ID NO 1652) |
| | | MRDVILFEK |
| | | (SEQ ID NO 1653) |
| | | MRDVILFEK ox |
| | | (SEQ ID NO 1654) |
| | | NLEENYCR cam |
| | | (SEQ ID NO 1655) |
| | | NPDGDKGPWCYTTDPSVR cam |
| | | (SEQ ID NO 1656) |
| | | NPDGDVNGPWCYTTNPR cam |
| | | (SEQ ID NO 1657) |
| | | NPDGETAPWCYTTDSQLR cam |
| | | (SEQ ID NO 1658) |
| | | NPDNDEQGPWCYTTDPDKR cam |
| | | (SEQ ID NO 1659) |
| | | NYCRNPDGDVNGPWCYTTNPR cam cam |
| | | (SEQ ID NO 1660) |
| | | QLAAGGVSDCLAKCEGETDFVCR cam cam cam |
| | | (SEQ ID NO 1661) |
| | | TAVTAAGTPCQGWAAQEPHR cam |
| | | (SEQ ID NO 1662) |
| | | TICYITGWGETQGTFGAGR cam |
| | | (SEQ ID NO 1663) |
| | | TPENFPDAGLEMNYCR cam |
| | | (SEQ ID NO 1664) |
| | | TPENFPDAGLEMNYCR ox cam |
| | | (SEQ ID NO 1665) |
| | | VCNRVEYLNNR cam |
| | | (SEQ ID NO 1666) |
| | | VILGAHEEYIR |
| | | (SEQ ID NO 1667) |
| | | VILGAHEEYIRGLDVQEISVAK |
| | | (SEQ ID NO 1668) |
| | | VIPACLPSPNYMVADR cam |
| | | (SEQ ID NO 1669) |
| | | VIPACLPSPNYMVADR cam ox |
| | | (SEQ ID NO 1670) |
| | | VSRFVDWIER |
| | | (SEQ ID NO 1671) |
| | | WEYCDIPR cam |
| | | (SEQ ID NO 1672) |
| | | WEYCNLKR cam |
| | | (SEQ ID NO 1673) |
| | | WSEQTPHRHNR |
| | | (SEQ ID NO 1674) |
| gi\|6680608 | Pregnancy zone | AAPLSLCALTAVDQSVLLLKPEAK cam |
| | protein | (SEQ ID NO 1675) |
| | | AESPVFVQTDKPIYKPGQIVKFR |
| | | (SEQ ID NO 1676) |
| | | AINYLISGYQR |
| | | (SEQ ID NO 1677) |
| | | ALLAYAFALAGNK |
| | | (SEQ ID NO 1678) |
| | | ALLAYAFALAGNKAK |
| | | (SEQ ID NO 1679) |
| | | AMGVPMMGLDYSDEINQVVEVR ox ox |
| | | (SEQ ID NO 1680) |
| | | EEGTGIELTGIGSCEIANALSKLK cam |
| | | (SEQ ID NO 1681) |
| | | HSLGDNDAHSIFQSVGINIFTNSK |
| | | (SEQ ID NO 1682) |
| | | HSLGDNDAHSIFQSVGINIFTNSKIHKPR |
| | | (SEQ ID NO 1683) |
| | | LSPQSIYNLLPGK |
| | | (SEQ ID NO 1684) |
| | | SKAINYLISGYQR |
| | | (SEQ ID NO 1685) |
| | | SVIVEPEGIEK |
| | | (SEQ ID NO 1686) |
| | | VKALSFYQPR |
| | | (SEQ ID NO 1687) |
| | | VNTNYRPGLPFSGQVLLVDEK |
| | | (SEQ ID NO 1688) |
| gi\|53787 | Properdin (AA 5- | CGGHCPGEAQQSQACDTQK cam cam cam |
| | 441) | (SEQ ID NO 1689) |
| | | DIRVEDCCLNAAYAFQEHDGGLCQACR cam cam |
| | | cam cam |
| | | (SEQ ID NO 1690) |
| | | HCYNIHNCIMK cam cam |
| | | (SEQ ID NO 1691) |
| | | HCYNIHNCIMK cam cam ox |
| | | (SEQ ID NO 1692) |
| | | HGGPFCAGDATR cam (SEQ ID NO 1693) |
| | | HGGPFCAGDATRNQMCNK cam ox cam |
| | | (SEQ ID NO 1694) |
| | | LQDIRHCYNIHNCIMK cam cam |
| | | (SEQ ID NO 1695) |
| | | LQDIRHCYNIHNCIMK cam cam ox |
| | | (SEQ ID NO 1696) |
| | | LRMSINCEGTPGQQSR cam |
| | | (SEQ ID NO 1697) |
| | | LRMSINCEGTPGQQSR ox cam |
| | | (SEQ ID NO 1698) |
| | | MSINCEGTPGQQSR cam |
| | | (SEQ ID NO 1699) |
| | | MSINCEGTPGQQSR ox cam |
| | | (SEQ ID NO 1700) |
| | | QRLCTPLLPK cam |
| | | (SEQ ID NO 1701) |
| | | QRVCDNPAPK cam |
| | | (SEQ ID NO 1702) |
| | | SCSAPAPSHQPPGKPCSGPAYEHK cam cam |
| | | (SEQ ID NO 1703) |
| | | SRSCSAPAPSHQPPGKPCSGPAYEHK cam cam |
| | | (SEQ ID NO 1704) |
| gi\|49868 | Put. beta-actin (aa | DLYANTVLSGGTTMYPGIADR |
| | 27-375) | (SEQ ID NO 1705) |
| | | DLYANTVLSGGTTMYPGIADR ox |
| | | (SEQ ID NO 1706) |
| | | DSYVGDEAQSK |
| | | (SEQ ID NO 1707) |
| | | DSYVGDEAQSKR |
| | | (SEQ ID NO 1708) |
| | | GYSFTTTAER |
| | | (SEQ ID NO 1709) |
| | | GYSFTTTAEREIVR |
| | | (SEQ ID NO 1710) |
| | | HQGVMVGMGQK ox ox |
| | | (SEQ ID NO 1711) |
| | | IWHHTFYNELR |
| | | (SEQ ID NO 1712) |
| | | KDLYANTVLSGGTTMYPGIADR ox |
| | | (SEQ ID NO 1713) |
| | | LCYVALDFEQEMATAASSSSLEK cam ox |
| | | (SEQ ID NO 1714) |
| | | LDLAGRDLTDYLMK |
| | | (SEQ ID NO 1715) |
| | | LDLAGRDLTDYLMK ox |
| | | (SEQ ID NO 1716) |
| | | QEYDESGPSIVHR |
| | | (SEQ ID NO 1717) |
| | | QEYDESGPSIVHRK |
| | | (SEQ ID NO 1718) |
| | | SYELPDGQVITIGNER |
| | | (SEQ ID NO 1719) |
| | | VAPEEHPVLLTEAPLNPK |
| | | (SEQ ID NO 1720) |
| gi\|34785996 | Pzp protein | AAPLSLCALTAVDQSVLLLKPEAK cam |
| | | (SEQ ID NO 1721) |
| | | AESPVFVQTDKPIYKPGQIVK |
| | | (SEQ ID NO 1722) |
| | | AESPVFVQTDKPIYKPGQIVKFR |
| | | (SEQ ID NO 1723) |
| | | AFAQAQSHIFIEK |
| | | (SEQ ID NO 1724) |
| | | AINYLISGYQR |
| | | (SEQ ID NO 1725) |
| | | ALLAYAFALAGNK |
| | | (SEQ ID NO 1726) |
| | | APSAEVEMTAYVLLAYLTSESSRPTR ox |
| | | (SEQ ID NO 1727) |
| | | ATVLNYMSHCIQIR ox cam |
| | | (SEQ ID NO 1728) |
| | | DAVKEEDSLHWQRPGDVQK |
| | | (SEQ ID NO 1729) |
| | | DLTFYYLIK |
| | | (SEQ ID NO 1730) |
| | | EEGTGIELTGIGSCEIANALSKLK cam |
| | | (SEQ ID NO 1731) |
| | | ENGCFQQSGYLLNNAMK cam |
| | | (SEQ ID NO 1732) |
| | | ENGCFQQSGYLLNNAMK cam ox |
| | | (SEQ ID NO 1733) |
| | | GSGSGCVYLQTSLK cam |
| | | (SEQ ID NO 1734) |
| | | GSIFNLGSHVLSLEQGNMK |
| | | (SEQ ID NO 1735) |
| | | GSIFNLGSHVLSLEQGNMK ox |
| | | (SEQ ID NO 1736) |
| | | HSLGDNDAHSIFQSVGINIFTNSK |
| | | (SEQ ID NO 1737) |
| | | IHYLLNEDIMKNEK ox |
| | | (SEQ ID NO 1738) |
| | | KIEHSFEVK |
| | | (SEQ ID NO 1739) |
| | | KLQDQPNIQR |
| | | (SEQ ID NO 1740) |
| | | LPDLPGNYVTK |
| | | (SEQ ID NO 1741) |
| | | LSPQSIYNLLPGK |
| | | (SEQ ID NO 1742) |
| | | LTEVPALVHKDTVVK |
| | | (SEQ ID NO 1743) |
| | | NEKDLTFYYLIK |
| | | (SEQ ID NO 1744) |
| | | SKAINYLISGYQR |
| | | (SEQ ID NO 1745) |
| | | SQKEVLVTIESSGTFSK |
| | | (SEQ ID NO 1746) |
| | | TEVNTNHVLIYIEK |
| | | (SEQ ID NO 1747) |
| | | THITNAFNWLSMK ox |
| | | (SEQ ID NO 1748) |
| | | TVQGAFFGVPVYK |
| | | (SEQ ID NO 1749) |
| | | VKALSFYQPR |
| | | (SEQ ID NO 1750) |
| | | VNTNYRPGLPFSGQVLLVDEK |
| | | (SEQ ID NO 1751) |
| | | YNILPVADGK |
| | | (SEQ ID NO 1752) |
| gi\|33859612 | Retinol binding | DPNGLSPETR |
| | protein 4 | (SEQ ID NO 1753) |
| | | DPNGLSPETRR |
| | | (SEQ ID NO 1754) |
| | | FSGLWYAIAK |
| | | (SEQ ID NO 1755) |
| | | FSGLWYAIAKK |
| | | (SEQ ID NO 1756) |
| | | LLSNWEVCADMVGTFTDTEDPAKFK cam ox |
| | | (SEQ ID NO 1757) |
| | | LQNLDGTCADSYSFVFSR cam |
| | | (SEQ ID NO 1758) |
| | | LQNLDGTCADSYSFVFSRDPNGLSPETR cam |
| | | (SEQ ID NO 1759) |
| | | MKYWGVASFLQR |
| | | (SEQ ID NO 1760) |
| | | MKYWGVASFLQR ox |
| | | (SEQ ID NO 1761) |
| | | QEELCLER cam |
| | | (SEQ ID NO 1762) |
| | | QRQEELCLER cam |
| | | (SEQ ID NO 1763) |
| | | QYRWIEHNGYCQSRPSR cam |
| | | (SEQ ID NO 1764) |
| | | WIEHNGYCQSRPSR cam |
| | | (SEQ ID NO 1765) |
| | | YWGVASFLQR |
| | | (SEQ ID NO 1766) |
| gi\|6678083 | Serine (or cysteine) | AVLTMDETGTEAAAATVLLAVPYSMPPIVR |
| | proteinase inhibitor, | (SEQ ID NO 1767) |
| | clade A, member 1a | AVLTMDETGTEAAAATVLLAVPYSMPPIVR ox |
| | | (SEQ ID NO 1768) |
| | | AVLTMDETGTEAAAATVLLAVPYSMPPIVR ox ox |
| | | (SEQ ID NO 1769) |
| | | DQSPASHEIATNLGDFAISLYR |
| | | (SEQ ID NO 1770) |
| | | FDHPFLFIIFEEHTQSPLFVGK |
| | | (SEQ ID NO 1771) |
| | | FLEEAKNHYQAEVFSVNFAESEEAK |
| | | (SEQ ID NO 1772) |
| | | IFNNGADLSGITEENAPLK |
| | | (SEQ ID NO 1773) |
| | | IFNNGADLSGITEENAPLKLSQAVHK |
| | | (SEQ ID NO 1774) |
| | | KLDQDTVFALANYILFK |
| | | (SEQ ID NO 1775) |
| | | KPFDPENTEEAEFHVDESTTVK |
| | | (SEQ ID NO 1776) |
| | | LAQIHFPRLSISGEYNLK |
| | | (SEQ ID NO 1777) |
| | | LDQDTVFALANYILFK |
| | | (SEQ ID NO 1778) |
| | | LSISGEYNLK |
| | | (SEQ ID NO 1779) |
| | | MQHLEQTLSKELISK |
| | | (SEQ ID NO 1780) |
| | | NHYQAEVFSVNFAESEEAK |
| | | (SEQ ID NO 1781) |
| | | NHYQAEVFSVNFAESEEAKK |
| | | (SEQ ID NO 1782) |
| | | RLAQIHFPR |
| | | (SEQ ID NO 1783) |
| | | SFQHLLQTLNRPDSELQLSTGNGLFVNNDLK |
| | | (SEQ ID NO 1784) |
| | | SFQHLLQTLNRPDSELQLSTGNGLFVNNDLKLVEK |
| | | (SEQ ID NO 1785) |
| | | TLMSPLGITR ox |
| | | (SEQ ID NO 1786) |
| | | VINDFVEKGTQGK |
| | | (SEQ ID NO 1787) |
| | | WKKPFDPENTEEAEFHVDESTTVK |
| | | (SEQ ID NO 1788) |
| gi\|15029662 | Serine (or cysteine) | DQSPASHEIATNLGDFAISLYR |
| | proteinase inhibitor, | (SEQ ID NO 1789) |
| | clade A, member la | FDHPFLFIIFEEHTQSPIFVGK |
| | | (SEQ ID NO 1790) |
| | | IFNNGADLSGITEENAPLK |
| | | (SEQ ID NO 1791) |
| | | IFNNGADLSGITEENAPLKLSQAVHK |
| | | (SEQ ID NO 1792) |
| | | KLDQDTVFALANYILFK |
| | | (SEQ ID NO 1793) |
| | | KPFDPENTEEAEFHVDESTTVK |
| | | (SEQ ID NO 1794) |
| | | LDQDTVFALANYILFK |
| | | (SEQ ID NO 1795) |
| | | LSISGEYNLK |
| | | (SEQ ID NO 1796) |
| | | LSQAVHKAVLTIDETGTEAAAVTVLQMVPMSMPPILR |
| | | ox ox ox |
| | | (SEQ ID NO 1797) |
| | | MQHLEQTLSK |
| | | (SEQ ID NO 1798) |
| | | NHYQAEVFSVNFAESEEAK |
| | | (SEQ ID NO 1799) |
| | | NHYQAEVFSVNFAESEEAKK |
| | | (SEQ ID NO 1800) |
| | | RLAQIHFPR |
| | | (SEQ ID NO 1801) |
| | | TLMSPLGITR |
| | | (SEQ ID NO 1802) |
| | | TLMSPLGITR ox |
| | | (SEQ ID NO 1803) |
| | | VINDFVEKGTQGK |
| | | (SEQ ID NO 1804) |
| | | WKKPFDPENTEEAEFHVDESTTVK |
| | | (SEQ ID NO 1805) |
| gi\|6678085 | Serine (or cysteine) | DQSPASHEIATNLGDFALR |
| | proteinase inhibitor, | (SEQ ID NO 1806) |
| | clade A, member 1d | ELISQFLLNRR |
| | | (SEQ ID NO 1807) |
| | | FDHPFLFIIFEEHTQSPIFVGK |
| | | (SEQ ID NO 1808) |
| | | FLEEAKNHYQAEVFSVNFAESEEAK |
| | | (SEQ ID NO 1809) |
| | | IFNNGADLSGITEENAPLK |
| | | (SEQ ID NO 1810) |
| | | IFNNGADLSGITEENAPLKLSK |
| | | (SEQ ID NO 1811) |
| | | KLDQDTVFALANYILFK |
| | | (SEQ ID NO 1812) |
| | | KVINDFVEK |
| | | (SEQ ID NO 1813) |
| | | LDQDTVFALANYILFK |
| | | (SEQ ID NO 1814) |
| | | LSISGNYNLK |
| | | (SEQ ID NO 1815) |
| | | MQHLEQTLNKELISQFLLNR |
| | | (SEQ ID NO 1816) |
| | | NHYQAEVFSVNFAESEEAK |
| | | (SEQ ID NO 1817) |
| | | NHYQAEVFSVNFAESEEAKK |
| | | (SEQ ID NO 1818) |
| | | QPFDPENTEEAEFHVDESTTVK |
| | | (SEQ ID NO 1819) |
| | | RSDAQIHIPR |
| | | (SEQ ID NO 1820) |
| | | SDAQIHIPR |
| | | (SEQ ID NO 1821) |
| | | TLMSPLGITR |
| | | (SEQ ID NO 1822) |
| | | TLMSPLGITR ox |
| | | (SEQ ID NO 1823) |
| | | TLMSPLGITRIFNNGADLSGITEENAPLK ox |
| | | (SEQ ID NO 1824) |
| | | VINDFVEKGTQGK |
| | | (SEQ ID NO 1825) |
| | | WKQPFDPENTEEAEFHVDESTTVK |
| | | (SEQ ID NO 1826) |
| gi\|6678087 | Serine (or cysteine) | DQSPASHEIATNLGDFAISLYR |
| | proteinase inhibitor, | (SEQ ID NO 1827) |
| | clade A, member 1e | FLEEAKNHYQAEVFSVNFAESEEAK |
| | | (SEQ ID NO 1828) |
| | | IFNSGADLSGITEENAPLK |
| | | (SEQ ID NO 1829) |
| | | IFNSGADLSGITEENAPLKLSQAVHK |
| | | (SEQ ID NO 1830) |
| | | KLEQDTVFVLANYILFK |
| | | (SEQ ID NO 1831) |
| | | KPFDPENTK |
| | | (SEQ ID NO 1832) |
| | | KVINDFVEK |
| | | (SEQ ID NO 1833) |
| | | LAQIHIPRLSISGNYNLETLMSPLGITR |
| | | (SEQ ID NO 1834) |
| | | LAQIHIPRLSISGNYNLETLMSPLGITR ox |
| | | (SEQ ID NO 1835) |
| | | LEQDTVFVLANYILFK |
| | | (SEQ ID NO 1836) |
| | | LSISGNYNLETLMSPLGITR |
| | | (SEQ ID NO 1837) |
| | | LSISGNYNLETLMSPLGITR ox |
| | | (SEQ ID NO 1838) |
| | | MQHLEQTLNKELISK |
| | | (SEQ ID NO 1839) |
| | | MQHLEQTLNKELISK ox |
| | | (SEQ ID NO 1840) |
| | | NHYQAEVFSVNFAESEEAKK |
| | | (SEQ ID NO 1841) |
| | | QAEFHVDESTTVK |
| | | (SEQ ID NO 1842) |
| | | RLAQIHIPR |
| | | (SEQ ID NO 1843) |
| | | VINDFVEKGTQGK |
| | | (SEQ ID NO 1844) |
| | | WKKPFDPENTK |
| | | (SEQ ID NO 1845) |
| gi\|18044689 | Serine (or cysteine) | ALYQTEAFTADFQQPTEAK |
| | proteinase inhibitor, | (SEQ ID NO 1846) |
| | clade A, member 3K | AVLDVAETGTEAAAATGVIGGIR |
| | | (SEQ ID NO 1847) |
| | | AVLDVAETGTEAAAATGVIGGIRK |
| | | (SEQ ID NO 1848) |
| | | DLQILAEFHEK |
| | | (SEQ ID NO 1849) |
| | | FSIASNYR |
| | | (SEQ ID NO 1850) |
| | | HFRDEELSCSVLELK cam |
| | | (SEQ ID NO 1851) |
| | | ISFDPQDTFESEFYLDEKR |
| | | (SEQ ID NO 1852) |
| | | KTLFPSQIEELNLPK |
| | | (SEQ ID NO 1853 |
| | | LALKNPDTNIVFSPLSISAALALVSLGAK |
| | | (SEQ ID NO 1854) |
| | | MQQVEASLQPETLR ox |
| | | (SEQ ID NO 1855) |
| | | MQQVEASLQPETLRK ox |
| | | (SEQ ID NO 1856) |
| | | TLFPSQIEELNLPK |
| | | (SEQ ID NO 1857) |
| | | TLMVLVNYIYFK ox |
| | | (SEQ ID NO 1858) |
| gi\|18252782 | Serine (or cysteine) | ANRPFLVLIR |
| | proteinase inhibitor, | (SEQ ID NO 1859) |
| | clade C | ANRPFLVLIREVALNTIIFMGR ox |
| | (antithrombin), | (SEQ ID NO 1860) |
| | member 1 | ATEEDGSEQKVPEATNR |
| | | (SEQ ID NO 1861) |
| | | DIPVNPLCIYR cam |
| | | (SEQ ID NO 1862) |
| | | DIPVNPLCIYRSPGK cam |
| | | (SEQ ID NO 1863) |
| | | ENPEQSRVTINNWVANK |
| | | (SEQ ID NO 1864) |
| | | EQLQDMGLIDLFSPEK ox |
| | | (SEQ ID NO 1865) |
| | | EVALNTIIFMGR |
| | | (SEQ ID NO 1866) |
| | | EVALNTIIFMGR ox |
| | | (SEQ ID NO 1867) |
| | | FATNFYQHLADSK |
| | | (SEQ ID NO 1868) |
| | | FRTEDGFSLK |
| | | (SEQ ID NO 1869) |
| | | IKDVIPQGAINELTALVLVNTIYFK |
| | | (SEQ ID NO 1870) |
| | | LQPLDFKENPEQSR |
| | | (SEQ ID NO 1871) |
| | | NDNDNIFLSPLSISTAFAMTK ox |
| | | (SEQ ID NO 1872) |
| | | QLMEVFKFDTISEK |
| | | (SEQ ID NO 1873) |
| | | QLMEVFKFDTISEK ox |
| | | (SEQ ID NO 1874) |
| | | SKFSPENTR |
| | | (SEQ ID NO 1875) |
| | | SLNPNRVTFK |
| | | (SEQ ID NO 1876) |
| | | SQLPGIVAGGRDDLYVSDAFHK |
| | | (SEQ ID NO 1877) |
| | | SSDLVSANRLFGDK |
| | | (SEQ ID NO 1878) |
| | | TSDQIHFFFAK |
| | | (SEQ ID NO 1879) |
| | | VAEGTQVLELPFKGDDITMVLILPKPEK |
| | | (SEQ ID NO 1880) |
| | | VAEGTQVLELPFKGDDITMVLILPKPEK ox |
| | | (SEQ ID NO 1881) |
| | | VDGQSCPVPMMYQEGK cam ox ox |
| | | (SEQ ID NO 1882) |
| | | VDGQSCPVPMMYQEGK ox cam |
| | | (SEQ ID NO 1883) |
| gi\|6679383 | Serine (or cysteine) | DFFHLDER |
| | proteinase inhibitor, | (SEQ ID NO 1884) |
| | clade F, member 2 | FDPSLTQKDFFHLDER |
| | | (SEQ ID NO 1885) |
| | | FTVSVDMMHAVSYPLR ox |
| | | (SEQ ID NO 1886) |
| | | FTVSVDMMHAVSYPLR ox ox |
| | | (SEQ ID NO 1887) |
| | | GFPIKDDFLEQSER |
| | | (SEQ ID NO 1888) |
| | | LAPRMEEDYPQFSSPK ox |
| | | (SEQ ID NO 1889) |
| | | LDNQDFGDHATLK |
| | | (SEQ ID NO 1890) |
| | | LDNQDFGDHATLKR |
| | | (SEQ ID NO 1891) |
| | | LFGAKPVK |
| | | (SEQ ID NO 1892) |
| | | LIGQNDKADFHGGK |
| | | (SEQ ID NO 1893) |
| | | LPLPALFK |
| | | (SEQ ID NO 1894) |
| | | LTGKQEEDLANINQWVK |
| | | (SEQ ID NO 1895) |
| | | NPNPSALPQLQEQR |
| | | (SEQ ID NO 1896) |
| | | NPNPSALPQLQEQRDSPDNR |
| | | (SEQ ID NO 1897) |
| | | QEEDLANINQWVK |
| | | (SEQ ID NO 1898) |
| | | SVPTAEETRR |
| | | (SEQ ID NO 1899) |
| | | WFLLEQPEIQVAHFPFK |
| | | (SEQ ID NO 1900) |
| gi\|l5929675 | Serpina1a protein | AVLTIDETGTEAAAVTVLLAVPYSMPPILR |
| | | (SEQ ID NO 1901) |
| | | AVLTIDETGTEAAAVTVLLAVPYSMPPILR ox |
| | | (SEQ ID NO 1902) |
| | | DQSPASHEIATNLGDFAISLYR |
| | | (SEQ ID NO 1903) |
| | | FDHPFLFIIFEEHTQSPLFVGK |
| | | (SEQ ID NO 1904) |
| | | FLEEAKNHYQAEVFSVNFAESEEAK |
| | | (SEQ ID NO 1905) |
| | | IFNNGADLSGITEENAPLK |
| | | (SEQ ID NO 1906) |
| | | IFNNGADLSGITEENAPLKLSQAVHK |
| | | (SEQ ID NO 1907) |
| | | KLDQDTVFALANYILFK |
| | | (SEQ ID NO 1908) |
| | | KPFDPENTEEAEFHVDESTTVK |
| | | (SEQ ID NO 1909) |
| | | KVINDFVEK |
| | | (SEQ ID NO 1910) |
| | | LDQDTVFALANYILFK |
| | | (SEQ ID NO 1911) |
| | | LSISGEYNLK |
| | | (SEQ ID NO 1912) |
| | | MQHLEQTLSK |
| | | (SEQ ID NO 1913) |
| | | MQHLEQTLSK ox |
| | | (SEQ ID NO 1914) |
| | | NHYQAEVFSVNFAESEEAK |
| | | (SEQ ID NO 1915) |
| | | NHYQAEVFSVNFAESEEAKK |
| | | (SEQ ID NO 1916) |
| | | RLAQIHFPR |
| | | (SEQ ID NO 1917) |
| | | SFQHLLQTLNRPDSELQLSTGNGLFVNNDLK |
| | | (SEQ ID NO 1918) |
| | | SFQHLLQTLNRPDSELQLSTGNGLFVNNDLKLVEK |
| | | (SEQ ID NO 1919) |
| | | TLMSPLGITR |
| | | (SEQ ID NO 1920) |
| | | TLMSPLGITR ox |
| | | (SEQ ID NO 1921) |
| | | VINDFVEKGTQGK |
| | | (SEQ ID NO 1922) |
| | | WKKPFDPENTEEAEFHVDESTTVK |
| | | (SEQ ID NO 1923) |
| gi\|14602605 | Serpina1a protein | AVLTIDETGTEAAAVTVLLAVPYSMPPILR |
| | | (SEQ ID NO 1924) |
| | | AVLTIDETGTEAAAVTVLLAVPYSMPPILR ox |
| | | (SEQ ID NO 1925) |
| | | DQSPASHEIATNLGDFAISLYR |
| | | (SEQ ID NO 1926) |
| | | FDHPFLFIIFEEHTQSPLFVGK |
| | | (SEQ ID NO 1927) |
| | | FLEEAKNHYQAEVFSVNFAESEEAK |
| | | (SEQ ID NO 1928) |
| | | FLLNRR |
| | | (SEQ ID NO 1929) |
| | | IFNNGADLSGITEENAPLK |
| | | (SEQ ID NO 1930) |
| | | IFNNGADLSGITEENAPLKLSQAVHK |
| | | (SEQ ID NO 1931) |
| | | KLDQDTVFALANYILFK |
| | | (SEQ ID NO 1932) |
| | | KPFDPENTEEAEFHVDESTTVK |
| | | (SEQ ID NO 1933) |
| | | KVINDFVEK |
| | | (SEQ ID NO 1934) |
| | | LAQIHFPR |
| | | (SEQ ID NO 1935) |
| | | LDQDTVFALANYILFK |
| | | (SEQ ID NO 1936) |
| | | LSISGEYNLK |
| | | (SEQ ID NO 1937) |
| | | MQHLEQTLSK ox |
| | | (SEQ ID NO 1938) |
| | | NHYQAEVFSVNFAESEEAK |
| | | (SEQ ID NO 1939) |
| | | NHYQAEVFSVNFAESEEAKK |
| | | (SEQ ID NO 1940) |
| | | RLAQIHFPR |
| | | (SEQ ID NO 1941) |
| | | SFQHLLQTLNRPDSELQLSTGNGLFVNNDLK |
| | | (SEQ ID NO 1942) |
| | | TLMSPLGITR |
| | | (SEQ ID NO 1943) |
| | | TLMSPLGITR ox |
| | | (SEQ ID NO 1944) |
| | | TLMSPLGITRIFNNGADLSGITEENAPLK |
| | | (SEQ ID NO 1945) |
| | | VINDFVEK |
| | | (SEQ ID NO 1946) |
| | | VINDFVEKGTQGK |
| | | (SEQ ID NO 1947) |
| | | WKKPFDPENTEEAEFHVDESTTVK |
| | | (SEQ ID NO 1948) |
| gi\|38174334 | Serum amyloid P- | APPSIVLGQEQDNYGGGFQR |
| | component | (SEQ ID NO 1949) |
| | | EKVGEYSLYIGQSK |
| | | (SEQ ID NO 1950) |
| | | EYTVKAPPSIVLGQEQDNYGGGFQR |
| | | (SEQ ID NO 1951) |
| | | GRDNELLIYK |
| | | (SEQ ID NO 1952) |
| | | SQSLFSYSVKGR |
| | | (SEQ ID NO 1953) |
| | | TYSDLSRSQSLFSYSVK |
| | | (SEQ ID NO 1954) |
| | | VFVFPRESETDHVK |
| | | (SEQ ID NO 1955) |
| | | VGEYSLYIGQSK |
| | | (SEQ ID NO 1956) |
| | | VGEYSLYIGQSKVTVR |
| | | (SEQ ID NO 1957) |
| gi\|20330802 | Transferrin | ADRDQYELLCLDNTR cam |
| | | (SEQ ID NO 1958) |
| | | AVLTSQETLFGGSDCTGNFCLFK cam cam |
| | | (SEQ ID NO 1959) |
| | | AVSSFFSGSCVPCADPVAFPK cam cam |
| | | (SEQ ID NO 1960) |
| | | CAPNNKEEYNGYTGAFR cam |
| | | (SEQ ID NO 1961) |
| | | CFVKLPEGTTPEK cam |
| | | (SEQ ID NO 1962) |
| | | CLKDGGGDVAFVK cam |
| | | (SEQ ID NO 1963) |
| | | CLVEKGDVAFVK cam |
| | | (SEQ ID NO 1964) |
| | | DFASCHLAQAPNHVVVSR cam |
| | | (SEQ ID NO 1965) |
| | | DFASCHLAQAPNHVVVSRK cam |
| | | (SEQ ID NO 1966) |
| | | DFQLFSSPLGK |
| | | (SEQ ID NO 1967) |
| | | DGGGDVAFVK |
| | | (SEQ ID NO 1968) |
| | | DLLFKDSAFGLLR |
| | | (SEQ ID NO 1969) |
| | | DLLFRDDTK |
| | | (SEQ ID NO 1970) |
| | | DQYELLCLDNTR cam |
| | | (SEQ ID NO 1971) |
| | | DSAFGLLR |
| | | (SEQ ID NO 1972) |
| | | DSAFGLLRVPPR |
| | | (SEQ ID NO 1973) |
| | | FDEFFSQGCAPGYEK cam |
| | | (SEQ ID NO 1974) |
| | | GTDFQLNQLEGKK |
| | | (SEQ ID NO 1975) |
| | | GYYAVAVVK |
| | | (SEQ ID NO 1976) |
| | | HQTVLDNTEGKNPAEWAK |
| | | (SEQ ID NO 1977) |
| | | IPSHAVVAR |
| | | (SEQ ID NO 1978) |
| | | KPVKDFASCHLAQAPNHVVVSR cam |
| | | (SEQ ID NO 1979) |
| | | KSCHTGLGR cam |
| | | (SEQ ID NO 1980) |
| | | KSCHTGVDR cam |
| | | (SEQ ID NO 1981) |
| | | LCQLCPGCGCSSTQPFFGYVGAFK cam cam cam |
| | | cam |
| | | (SEQ ID NO 1982) |
| | | LLEACTFHKH cam |
| | | (SEQ ID NO 1983) |
| | | LYLGHNYVTAIR |
| | | (SEQ ID NO 1984) |
| | | NLKQEDFELLCPDGTR cam |
| | | (SEQ ID NO 1985) |
| | | NQQEGVCPEGSIDNSPVK cam |
| | | (SEQ ID NO 1986) |
| | | NQQEGVCPEGSIDNSPVKWCALSHLER cam cam |
| | | (SEQ ID NO 1987) |
| | | SAGWVIPIGLLFCK cam |
| | | (SEQ ID NO 1988) |
| | | SCHTGLGR cam |
| | | (SEQ ID NO 1989) |
| | | SCHTGVDR cam |
| | | (SEQ ID NO 1990) |
| | | SKDFQLFSSPLGK |
| | | (SEQ ID NO 1991) |
| | | TAGWNIPMGMLYNR ox |
| | | (SEQ ID NO 1992) |
| | | TAGWNIPMGMLYNR ox ox |
| | | (SEQ ID NO 1993) |
| | | TSYPDCIK cam |
| | | (SEQ ID NO 1994) |
| | | TVLPPDGPR |
| | | (SEQ ID NO 1995) |
| | | VAQEHFGK |
| | | (SEQ ID NO 1996) |
| | | VPPRMDYR ox |
| | | (SEQ ID NO 1997) |
| | | WCALSHLER cam |
| | | (SEQ ID NO 1998) |
| | | WCAVSEHENTK cam |
| | | (SEQ ID NO 1999) |
| | | YLGAEYMQSVGNMR |
| | | (SEQ ID NO 2000) |
| | | YLGAEYMQSVGNMR ox |
| | | (SEQ ID NO 2001) |
| | | YLGAEYMQSVGNMRK |
| | | (SEQ ID NO 2002) |
| | | YLGAEYMQSVGNMRK ox |
| | | (SEQ ID NO 2003) |
| | | YLGAEYMQSVGNMRK ox ox |
| | | (SEQ ID NO 2004) |
| gi\|19354093 | Transthyretin | FVEGVYRVELDTK |
| | | (SEQ ID NO 2005) |
| | | GSPAVDVAVKVFK |
| | | (SEQ ID NO 2006) |
| | | HYTIAALLSPYSYSTTAVVSNPQN |
| | | (SEQ ID NO 2007) |
| | | KTSEGSWEPFASGK |
| | | (SEQ ID NO 2008) |
| | | LFLLCLAGLVFVSEAGPAGAGESKCPLMVK cam |
| | | cam ox |
| | | (SEQ ID NO 2009) |
| | | TAESGELHGLTTDEK |
| | | (SEQ ID NO 2010) |
| | | TAESGELHGLTTDEKFVEGVYR |
| | | (SEQ ID NO 2011) |
| | | TLGISPFHEFADVVFTANDSGHR |
| | | (SEQ ID NO 2012) |
| | | TSEGSWEPFASGK |
| | | (SEQ ID NO 2013) |
| | | VELDTKSYWK |
| | | (SEQ ID NO 2014) |
| | | VLDAVRGSPAVDVAVK |
| | | (SEQ ID NO 2015) |
| gi\|111243 | Vitamin D-binding | CCESTSEDCMASELPEHTIK cam cam cam ox |
| | protein | (SEQ ID NO 2016) |
| | | DLCGQSTTQAMDQYTFELSR cam |
| | | (SEQ ID NO 2017) |
| | | DLCGQSTTQAMDQYTFELSR cam ox |
| | | (SEQ ID NO 2018) |
| | | DLCGQSTTQAMDQYTFELSRR cam |
| | | (SEQ ID NO 2019) |
| | | DLCGQSTTQAMDQYTFELSRR cam ox |
| | | (SEQ ID NO 2020) |
| | | ECCDTQDSVACFSTQSPLLKR cam cam cam |
| | | (SEQ ID NO 2021) |
| | | EVVSLTEECCAEGADPTCYDTR cam cam cam |
| | | (SEQ ID NO 2022) |
| | | FSSSTFEQVNQLVK |
| | | (SEQ ID NO 2023) |
| | | GFADQFLYEYSSNYGQAPLPLLVAYTK |
| | | (SEQ ID NO 2024) |
| | | GQEMCADYSENTFTEYK cam |
| | | (SEQ ID NO 2025) |
| | | GQEMCADYSENTFTEYKK ox cam |
| | | (SEQ ID NO 2026) |
| | | HLSLLTTMSNR |
| | | (SEQ ID NO 2027) |
| | | HLSLLTTMSNR ox |
| | | (SEQ ID NO 2028) |
| | | KFSSSTFEQVNQLVK |
| | | (SEQ ID NO 2029) |
| | | KLCMAALSHQPQEFPTYVEPTNDEICEAFR cam ox |
| | | cam |
| | | (SEQ ID NO 2030) |
| | | LAQKVPTANLENVLPLAEDFTEILSR |
| | | (SEQ ID NO 2031) |
| | | LPTGKDLCGQSTTQAMDQYTFELSR cam ox |
| | | (SEQ ID NO 2032) |
| | | LQMKHLSLLTTMSNR ox |
| | | (SEQ ID NO 2033) |
| | | NYLSMVGSCCTSANPTVCFVK cam cam cam |
| | | (SEQ ID NO 2034) |
| | | NYLSMVGSCCTSANPTVCFVK ox cam cam cam |
| | | (SEQ ID NO 2035) |
| | | QLTSFIEKGQEMCADYSENTFTEYK ox cam |
| | | (SEQ ID NO 2036) |
| | | RTQVPEVFLSK |
| | | (SEQ ID NO 2037) |
| | | SCESDAPFPVHPGTPECCTK cam cam cam |
| | | (SEQ ID NO 2038) |
| | | SCESDAPFPVHPGTPECCTKEGLER cam cam cam |
| | | (SEQ ID NO 2039) |
| | | SLSLILYSR |
| | | (SEQ ID NO 2040) |
| | | TLRECCDTQDSVACFSTQSPLLK cam cam cam |
| | | (SEQ ID NO 2041) |
| | | TQVPEVFLSK |
| | | (SEQ ID NO 2042) |
| | | VCSQYAAYGK cam |
| | | (SEQ ID NO 2043) |
| | | VCSQYAAYGKEK cam |
| | | (SEQ ID NO 2044) |
| | | VPTANLENVLPLAEDFTEILSR |
| | | (SEQ ID NO 2045) |
| gi\|1083568 | Zinc-alpha 2- | AREEIFLVTLK |
| | glycoprotein | (SEQ ID NO 2046) |
| | | AYLEEECPEMLKR cam |
| | | (SEQ ID NO 2047) |
| | | AYLEEECPEMLKR cam ox |
| | | (SEQ ID NO 2048) |
| | | CLAYGFYPQR cam |
| | | (SEQ ID NO 2049) |
| | | FQATAFLNDQAFFHYNSNSGK |
| | | (SEQ ID NO 2050) |
| | | GFSQSLSVQWDR |
| | | (SEQ ID NO 2051) |
| | | SHLDRIDPPTVTITSR |
| | | (SEQ ID NO 2052) |
| | | VIPGGNRIFK |
| | | (SEQ ID NO 2053) |
| | | WEAEKVYVQR |
| | | (SEQ ID NO 2054) |
| | | YAYDGEDFIEFNK |
| | | (SEQ ID NO 2055) |

Legenda
ox = methionin oxidation
cam = carbamidomethyl oxidation

### REFERENCES

(1) Zerbini, A.; Pilli, M.; Ferrari, C.; Missale, G. Dig. Liver Dis. 2006, 38(4), 221-5.
(2) Jemal, A.; Murray, T.; Ward, E.; Samuels, A.; Tiwari, R. C.; Ghafoor, A.; Feuer, E. J.; Thun, M. J. CA Cancer J. Clin. 2005, 55(1), 10-30.
(3) Chen, C. J.; Yu, M. W.; Liaw, Y. F. J. Gastroenterol. Hepatol. 1997, 12(9-10), S294-308.
(4) Santella, R. M.; Zhang, Y. J.; Young, T. L.; Lee, B. M.; Lu, X. Q. Adv. Exp. Med. Biol. 1991, 283,165-81.
(5) Chen, S. Y.; Wang, L. Y.; Lunn, R. M.; Tsai, W. Y.; Lee, P. H.; Lee, C. S.; Ahsan, H.; Zhang, Y. J.; Chen, C. J.; Santella, R. M. Int J Cancer 2002, 99(1), 14-21.
(6) Grizzi, F.; Franceschini, B.; Hamrick, C.; Frezza, E. E.; Cobos, E.; Chiriva-Internati, M. J. Transl. Med. 2007, 53
(7) Zhou, L.; Liu, J.; Luo, F. World J Gastroenterol 2006, 12(8), 1175-1181.
(8) Borlak, J.; Meier, T.; Halter, R.; Spanel, R.; Spanel-Borowski, K. Oncogene 2005, 24(11), 1809-1819.
(9) Sebastian, S.; Settleman, J.; Reshkin, S. J.; Azzariti, A.; Bellizzi, A.; Paradiso, A. Biochim. Biophys. Acta 2006, 1766(1), 120-139.
(10) Yano, S.; Kondo, K.; Yamaguchi, M.; Richmond, G.; Hutchison, M.; Wakeling, A.; Averbuch, S.; Wadsworth, P. Anticancer Res 2003, 23(5A), 3639-3650.
(11) Tonjes, R. R.; Lohler, J.; O'Sullivan, J. F.; Kay, G. F.; Schmidt, G. H.; Dalemans, W.; Pavirani, A.; Paul, D. Oncogene 1995, 10(4), 765-8.
(12) Shen, H; Cheng, G; Fan, H; Zhang, J; Zhang, X; Lu, H; Liu, C; Sun, F; Jin, H; Xu, X; Xu, G; Wang, S; Fang, C; Bao, H; Wang, Y; Wang, J; Zhong, H; Yu, Z; Liu, Y; Tang, Z; Yang, P. Proteomics, 2006, 6(2), 528-37.
(13) Cui, F; Wang, Y; Wang, J; Wei, K; Hu, J; Liu, F; Wang, H; Zhao, X; Zhang, X; Yang, X. Proteomics, 2006, 6(2), 498-504.
(14) Wisniewski, T.; Golabek, A. A.; Kida, E.; Wisniewski, K. E.; Frangione, B. Am J Pathol. 1995, 147(2), 238-244.
(15) Gerner, C.; Steinkellner, W.; Holzmann, K.; Gsur, A.; Grimm, R.; Ensinger, C.; Obrist, P.; Sauermann, G. Thromb Haemost. 2001, 85(3), 494-501.
(16) Vejda, S.; Posovszky, C.; Zelzer, S.; Peter, B.; Bayer, E.; Gelbmann, D.; Schulte-Hermann, R.; Gerner, C. Mol Cell Proteomics. 2002, 1(5), 387-393.
(17) Fan, B. L.; Zhu, W. L.; Zou, G. L.; Luo, G. S.; Xu, C. L.; Zhao, W. X. Ai. Zheng. 2004, 23(3), 249-253.
(18) Vassalli, J. D.; Sappino, A. P.; Belin, D. J Clin Invest 1991, 88 (4), 1067-1072.
(19) Sudhoff, T.; Schneider, W. Clin Investig. 1992, 70(8), 631-636.
(20) Brunner, G.; Preissner, K. T. Blood Coagul. Fibrinolysis 1994, 5(4), 625-639.
(21) Falleti, E.; Pirisi, M.; Fabris, C.; Bortolotti, N.; Soardo, G.; Toniutto, P.; Gonano, F.; Bartoli, E. Eur J Clin Chem. Clin Biochem. 1993, 31(7), 407-411.
(22) Perier, C.; Chamson, A.; Engler, R.; Frey, J. Clin Chem. 1983, 29 (1), 45-47.
(23) Chio, L. F.; Oon, C. J. Cancer 1979, 43(2), 596-604.
(24) Lichtor, T. Neurosci Lett. 1992, 138(2), 287-90.
(25) Tanne, J. H. BMJ. 1999, 319(7211), 662.
(26) Hough, C. D.; Sherman-Baust, C. A.; Pizer, E. S.; Montz, F. J.; Im, D. D.; Rosenshein, N. B.; Cho, K. R.; Riggins, G. J.; Morin, P. J. Cancer Res. 2000, 60(22), 6281-7.
(27) Niemi, M.; Kervinen, K.; Kiviniemi, H.; Lukkarinen, O.; Kyllonen, A. P.; Apaja-Sarkkinen, M.; Savolainen, M. J.; Kairaluoma, M. I.; Kesaniemi, Y. A. J Epidemiol Community Health. 2000, 54(12), 938-9.
(28) Yokoyama, Y.; Kuramitsu, Y.; Takashima, M.; Iizuka, N.; Terai, S.; Oka, M.; Nakamura, K.; Okita, K.; Sakaida, I. Int J Oncol. 2006, 28(3), 625-631.
(29) Xu, N.; Hurtig, M.; Zhang, X. Y.; Ye, Q.; Nilsson-Ehle, P. Biochim. Biophys. Acta 2004, 1683(1-3), 33-37.
(30) Luo, G.; Zhang, X.; Nilsson-Ehle, P.; Xu, N. Lipids Health Dis 2004, 3 21.
(31) Richter, S.; Shih, D. Q.; Pearson, E. R.; Wolfrum, C.; Fajans, S. S.; Hattersley, A. T.; Stoffel, M. Diabetes 2003, 52(12), 2989-2995.
(32) Karlsson, H.; Leanderson, P.; Tagesson, C.; Lindahl, M. Proteomics. 2005, 5(5), 1431-1445.
(33) Ruiu, G.; Gambino, R.; Veglia, F.; Pagano, G.; Cassader, M. Clin Genet. 1997, 52(3), 167-172.
(34) Chiang, M. T.; Otomo, M. I.; Itoh, H.; Furukawa, Y.; Kimura, S.; Fujimoto, H. Lipids 1991, 26(1), 46-52.
(35) Averna, M.; Paravizzini, G.; Marino, G.; Lanteri, E.; Cavera, G.; Barbagallo, C. M.; Petralia, S.; Cavallaro, S.; Magro, G.; Grasso, S.; Notarbartolo, A.; Travali, S. Int J Clin Lab Res 1997, 27(3), 207-212.
(36) Okkels, H.; Rasmussen, T. E.; Sanghera, D. K.; Kamboh, M. I.; Kristensen, T. Eur J. Biochem. 1999, 259(1-2), 435-440.
(37) Petersen, C. M.; Jensen, P. H.; Bukh, A.; Sunde, L.; Lamm, L. U.; Ingerslev, J. Scand J Clin Lab Invest 1990, 50(5), 479-485.
(38) Teng, H.; Zhang, W. Y.; Zhu, F. Q. Chin Med J (Engl.) 1994, 107(12), 910-914.
(39) Bhattacharya, M.; Barlow, J. J. Int Adv. Surg. Oncol. 1979, 2155-176.
(40) Bierl, C.; Voetsch, B.; Jin, R. C.; Handy, D. E.; Loscalzo, J. J Biol. Chem. 2004, 279(26), 26839-26845.
(41) Giannattasio, A.; De Rosa, M.; Smeraglia, R.; Zarrilli, S.; Cimmino, A.; Di Rosario, B.; Ruggiero, R.; Colao, A.; Lombardi, G. J Endocrinol Invest 2002, 25(11), 983-986.
(42) Burk, R. F.; Early, D. S.; Hill, K. E.; Palmer, I. S.; Boeglin, M. E. Hepatology 1998, 27(3), 794-8.
(43) Reszka, E.; Gromadzinska, J.; Stanczyk, M.; Wasowicz, W. Biol Trace Elem. Res 2005, 104(2), 165-172.
(44) Chignard, N; Shang, S; Wang, H; Marrero, J; Bréchot, C; Hanash, S; Beretta L. Gastroenterology, 2006, 130(7), 2010-22.
(45) Lauer, G.; Sollberg, S.; Cole, M.; Flamme, I.; Sturzebecher, J.; Mann, K.; Krieg, T.; Eming, S. A. J Invest Dermatol. 2000, 115(1), 12-18.
(46) Bhat, V B; Choi, M H; Wishnok, J S; Tannenbaum, S R. J Proteome Res., 2005, 4(5), 1814-25.
(47) Andersson, C.; Gelin, J.; Iresjo, B. M.; Lundholm, K. J. Surg. Res 1993, 55(6), 607-614.
(48) Van Molle, W.; Libert, C.; Fiers, W.; Brouckaert, P. J Immunol 1997, 159(7), 3555-3564.
(49) Agrawal, R. S.; Karhu, K.; Laukkanen, J.; Kirkinen, P.; Yla-Herttuala, S.; Agrawal, Y. P. Gene Ther. 1999, 6(1), 146-148.
(50) De Masi, S.; Tosti, M. E.; Mele, A. Dig Liver Dis. 2005, 37(4), 260-8.

The characteristics of the invention being disclosed in the preceding description, the subsequent drawings and claims can be of importance both singularly and in arbitrary combination for the implementation of the invention in its different embodiments.

The foregoing description of preferred embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A biomarker selected from a first group consisting of
Amy 1, Apo Al, Carbx, Casp, AFP, ApoM, SAP, Fib-a, Fib-b, Fib-g, ApoE, A2MG, A2MG isoform, Serpin, Clusterin, MHC-fB, SAP isoform,
or from a second group consisting of
Gpx3, properidin, MUP1, HMW-K, Lifr-p, Orm 1, MBL-A, MBP-C,
wherein the biomarker is regulated by EGF overexpression in a subject.

2. Biomarker as claimed in claim 1 selected from a first group consisting of
Amy 1, Apo Al, Carbx, Casp, Fib-a, Fib-b, Fib-g, Clusterin, MHC-fB, SAP isoform or from a second group consisting of
HMW-K, Lifr-p, Orm 1, MBL-A, MBP-C.

3. Biomarker as claimed in claim 1 selected from a first group consisting of
AFP, ApoE, ApoM,
or from a second group consisting of
Gpx3, A2MG, A2MG isoform, SAP.

4. A composition for qualifying the EGFR kinase activity in a subject suffering from or being susceptible to cancer, in particular by an in vitro body fluid analysis, comprising an effective amount of at least one biomarker selected from the first group according to one of the claims 1-3 or an effective amount of at least one biomarker selected from the second group according to one of the claims 1-3.

5. Composition as claimed in claim 4 comprising an effective amount of at least one biomarker selected from the first group according to one of the claims 1-3 and an effective amount of at least one biomarker selected from the second group according to one of the claims 1-3.

6. Use of a composition as claimed in one of the claims 4-5 for the production of a diagnostic agent, in particular of a diagnostic standard for body fluid analysis.

7. Use as claimed in claim 6 for the production of a diagnostic agent for qualifying the EGFR kinase activity in a subject suffering from or being susceptible to cancer, in particular cancer of the liver, lung, breast, colon, prostate, bladder, head and neck, ovary or brain.

8. Use as claimed in one of the claims 6-7 for the production of a diagnostic agent for predicting or monitoring the response of a cancer patient to a method of treating cancer comprising administering an EGFR kinase modulator.

9. A kit for qualifying the EGFR kinase activity in a subject suffering from or being susceptible to cancer, in particular for predicting or monitoring the response of a cancer patient to a method of treating cancer comprising administering an EGFR kinase modulator, comprising
at least one standard (1) indicative of the body fluid level of a biomarker selected from the first group according to one of the claims 1-3 in normal individuals or individuals having cancer associated with increased EGFR kinase activity and/or at least one standard (2) indicative of the body fluid level of a biomarker selected from the second group according to one of the claims 1-3 in normal individuals or individuals having cancer associated with increased EGFR kinase activity, and instructions for the use of the kit.

10. The kit as claimed in claim 9, wherein the at least one standard (1) comprises an indicative amount of at least one biomarker selected from the first group according to one of the claims 1-3 and/or wherein the at least one standard (2) comprises an indicative amount of at least one biomarker selected from the second group according to one of the claims 1-3.

11. The kit as claimed in one of the claims 9-10, comprising a mixture of the at least one standard (1) and the at least one standard (2), in particular the composition according to claim 5.

12. The kit as claimed in one of the claims 9-11, further comprising a lysis buffer according to one of the claims 23-25 and/or a digesting buffer according to one of the claims 23-25.

13. The kit as claimed in one of the claims 9-12, further comprising at least one antibody specific for a biomarker selected from the first group according to one of the claims 1-3 and/or at least one antibody specific for a biomarker selected from the second group according to one of the claims 1-3, and reagents effective to detect said biomarker(s) in a serum sample.

14. The kit as claimed in claim 13, wherein the at least one antibody is polyclonal.

15. The kit as claimed in one of the claims 13-14 comprising at least one labelled secondary antibody specific for the at least one antibody of claim 9-10.

16. A method of qualifying the EGFR kinase activity in a subject, comprising determining in a body fluid sample of a subject suffering from or being susceptible to cancer at least one biomarker selected from the first group according to one of the claims 1-3 and/or at least one biomarker selected from the second group according to one of the claims 1-3,
wherein the body fluid level of the at least one biomarker of said first group being significantly higher and/or the body fluid level of the at least one biomarker of said second group being significantly lower than the level of said biomarker(s) in the body fluid of subjects without cancer associated with increased activity of EGFR is indicative of induced EGFR kinase activity in the subject.

17. Method as claimed in claim 16 for predicting the response of a cancer patient to a method of treating cancer comprising administering an EGFR kinase modulator, wherein the body fluid level of the at least one biomarker of said first group being significantly higher and/or the body fluid level of the at least one biomarker of said second group being significantly lower than the level of said biomarker(s) in the body fluid of subjects without cancer associated with increased activity of EGFR is indicative that the subject will respond therapeutically to a method of treating cancer comprising administering an EGFR kinase modulator.

18. Method as claimed in claim 16 for monitoring the therapeutically response of a cancer patient to a method of treating cancer comprising administering an EGFR kinase modulator, wherein the body fluid level of the at least one biomarker of said first group before and after the treatment and/or the body fluid level of the at least one biomarker of said second group before and after the treatment is determined, and a significant decrease of said body fluid level(s) of the at least one biomarker of said first group and/or a significant increase of said body fluid level(s) of the at least one biomarker of said second group after the treatment is indicative that the cancer patient therapeutically responds to the administration of the EGFR kinase modulator.

19. The method as claimed in one of the claims 16-18, wherein an immunoassay is performed, in particular by using the kit as claimed in one of the claims 9-15.

20. The method as claimed in claim 19, wherein at least one antibody specific for a biomarker selected from the first group according to claim 2 and/or at least one antibody specific for a biomarker selected from the second group according to claim 2, and reagents effective to detect said biomarker(s) in a serum sample is used for the immunoassay.

21. The method as claimed in one of the claims 16-18, wherein a peptide mass fingerprinting is performed, in particular by using the kit as claimed in one of the claims 9-12.

22. The method as claimed in claim 21, comprising the steps of
isolating a serum sample from a blood sample of a subject suffering from or being susceptible to cancer;
adding lysis buffer to the serum sample;
separating the proteins of the lysed serum sample by 2-DE gel electrophoresis;
excising from the gel at least one sample containing a protein of interest;
adding digesting buffer to the at least one excised sample;
determining the amount of the at least one protein of interest by analyzing the at least one digest mixture by mass spectrometry.

23. The method as claimed in claim 22, wherein
the subject is a human patient or non-human transgenic animal; and/or
the serum sample is isolated by centrifuging the blood sample; and/or
the 2-DE is performed by using two different pH gradients; and/or
the lysis buffer comprises (a) at least one buffer component, (b) at least one chaotrope, (c) at least one detergens, (d) at least one reducing agent (e) at least one carrier ampholyte, (f) at least one ribonuclease; and/or
the protein of interest is a biomarker selected from the first group according to one of the claims 1-3 or a biomarker selected from the second group according to one of the claims 1-3; and/or
the digesting buffer comprises a bicarbonate compound and a protease; and/or
wherein the mass spectrometry is selected from the group consisting of MALDI-TOF and ESI-TOF.

24. The method as claimed in one of the claims 22-23, wherein
the subject is suffering from or is susceptible to cancer in particular cancer of the liver, lung, breast, colon, prostate, bladder, head and neck, ovary or brain; and/or
the 2-DE is performed by using the pH gradients 3-10 and 4-7; and/or
the lysis buffer is an aqueous solution of (a) at least one buffer compound selected from the group consisting of Tris and HEPES, (b) at least one chaotrope selected from the group consisting of urea and thiourea, (c) at least one detergens selected from the group consisting of CHAPS and SDS, (d) at least one reducing agent selected from the group consisting of DTT and TCEP, (e) at least one carrier ampholyte selected from the group consisting of biolyte 5-7 and biolyte 3-10, (f) at least one ribonuclease selected from the group consisting of endonuclease and exonuclease; and/or
the protein of interest is a biomarker selected from the first group according to one of the claims 2-3 or a biomarker selected from the second group according to one of the claims 2-3; and/or
the digesting buffer is an aqueous solution of at least one bicarbonate compound selected from the group consisting of ammonium bicarbonate and sodium bicarbonate and of at least one serine protease, in particular selected from the group consisting of trypsin, chymotrypsin and elastase; and/or
the mass spectrometry is performed by MALDI-TOF; and/or
a tandem mass spectrometer is used; and/or
a matrix is used for the mass spectrometry selected from the group consisting of 3,5-dimethoxy-4-hydroxycinnamic acid, α-cyano-4-hydroxycinnamic acid and 2,5-dihydroxybenzoic acid.

25. The method as claimed in one of the claims 22-24, wherein
the subject is a transgenic mouse, in particular a mouse whose genome comprises a non natural IgEGF sequence; and/or
the lysis buffer is an aqueous solution of (a) Tris; (b) urea and thiourea, (c) CHAPS, (d) DTT, (e) biolyte 3-10, (f) endonuclease; and/or
the serum sample is calibrated or the serum samples are equilibrated to a predefined protein concentration by adding the lysis buffer; and/or
the protein of interest is a biomarker selected from the first group according to claim 3 or a biomarker selected from the second group according to claim 3; and/or
the digesting buffer is an aqueous solution of ammonium bicarbonate and trypsin; and/or
a MALDI-TOF/TOF spectrometry is performed; and/or
a matrix is used for the mass spectrometry selected from the group consisting of α-cyano-4-hydroxycinnamic acid.

26. The method as claimed in one of the claims 22-25, further comprising the steps of
determining the protein concentration of the serum sample, in particular by the Bradford method; and/or
freezing and thawing the serum sample before the lysis buffer is added; and/or
staining the gel after the 2-DE, in particular by using coomassie blue; and/or
destaining the exised sample; and/or
shrinking, in particular by adding acetonitrile, and drying of the excised sample before the digesting buffer is added; and/or
using a peptide calibration standard for the mass spectrometry.

27. A procedure to screen for and to identify drugs against cancer associated with an increased EGFR kinase activity comprising determining in a body fluid sample of a transgenic cancer mouse being treated with a compound to be tested, in particular of a mouse whose genome comprises a non natural IgEGF sequence, at least one biomarker selected from the first group according to one of the claims 1-3 and/or at least one biomarker selected from the second group according to one of the claims 1-3, wherein the body fluid level of the at least one biomarker of said first group being significantly lower and/or the body fluid level of the at least one biomarker of said second group being significantly higher than the level of said biomarker(s) in the body fluid of an untreated transgenic cancer mouse is indicative of the therapeutic effect of said compound as a EGFR kinase modulator.

28. The procedure as claimed in claim 27, wherein the method as claimed in claim 18, in particular according to one of the claims 19-26, is used.
